SEQUENCE LISTING (24 sequences)

SEQ ID NO: 1 - protein, 234 aa, Homo sapiens
MGAFCQRPSSDKEQEKEKKSVICVEGNIASGKTTCLEFFSNATDVEVLTEPVSKWRNVRGHNPLGLMYHDASRWGLTLQTYVQLTMLDRHTRPQVSSVRLMERSIHSARYIFVENLYRSGKMPEVDYVVLSEWFDWILRNMDVSVDLIVYLRTNPETCYQRLKKRCREEEKVIPLEYLEAIHHLHEEWLIKGSLFPMAAPVLVIEADHHMERMLELFEQNRDRILTPENRKHCP

SEQ ID NO: 2 - DNA, 4678 bp, Homo sapiens
  source 1..4678: mol_type = other DNA
gccaagttatgggtgcgttctgccagcgtcctagcagtgataaagaacaggaaaaagagaaaaaatcagtgatctgtgtcgagggcaatattgcaagtgggaagacgacatgcctggaattcttctccaacgcgacagacgtcgaggtgttaacggagcctgtgtccaagtggagaaatgtccgtggccacaatcctctgggcctgatgtaccacgatgcctctcgctggggtcttacgctacagacttatgtgcagctcaccatgctggacaggcatactcgtcctcaggtgtcatctgtacggttgatggagaggtcgattcacagcgcaagatacatttttgtagaaaacctgtatagaagtgggaagatgccagaagtggactatgtagttctgtcggaatggtttgactggatcttgaggaacatggacgtgtctgttgatttgatagtttaccttcggaccaatcctgagacttgttaccagaggttaaagaagagatgcagggaagaggagaaggtcattccgctggaatacctggaagcaattcaccatctccatgaggagtggctcatcaaaggcagccttttccccatggcagcccctgttctggtgattgaggctgaccaccacatggagaggatgttagaactctttgaacaaaatcgggatcgaatattaactccagagaatcggaagcattgcccataggaggcaaaaggtctatggctcatgtctgaaaaatgcctgctgctgccaagttagctattgggagcaatctggaaaaacttgctcccaggagggctttgtgtctggccagcttgattttcctaatggtctcatctcctttgctagtgtctttgtcatgcgtctctggccctcgtgggtaaatgacaaacgggaccaatgggtttgccaagccctttgctgttcgcagcctcacattcccccggtgcctctcccatggctttgtgctgctgagtcgctctcatgaagcccttagggagagcacctgttgtgtgcctgacaccacgctggagctgtgtaccaatcgtctcagccttcattaggaggccgaggtaggagtcttatatcccaggtgaggaatttgaagctcagaaaggttgaggggctccccagaggtcacacagcctgtgtgcagtggagctggcaccattcagactttcagccgactcagcaactttcccttgccctgggctgcctcctcctgagagctgttccccaccgccctgcctcttccggttggaggctctcatgtctctttggggagagctggcagtgtgcggagctgataacattttcccaatattgagcagttcccaaggacagtcagcatttctagacttccacaaaattatgctgcatttggctggagcccggtgttcagtggtttccctgcccgaggtcgctgcagccccatctaccacatcttcatgtggacattgagattcacatgctggctcctgaagggtgctcagtctccttggtgattaaggtcctgcttgaactgctgccaactccatgtcagggaagtcgcttttggtgcctggctggtttgcccagagccaagctggggcaaggggcagccagccctggcttccaaggctcccgtactgtctgtgtccttgtataaggagctttgctcttggaattactgaaagtctgtggcccaagagagagacacaagtggccttaagtctttttgaagtgttatttcatccagggaaatgcctcgagccatagagcctgaaatcatctttgttggctcagaaaataccttagcttcactcagctggactgcattgaaggcgaggctgccccttggatcaagcagaaaacaagagaaagaaagaacgttccctttggggatagtctggaaagttgggatttgcaaataaaggctctggaagcattgctggtcctgaagctttggaggtgggcagagagagcttcaagaagactagatgcaaaccctggaaaggattaaggctcaactctggagaaacaggccacagcctctcagagcagctgttggctgtaaatagaggtagcaaggccgctcccaggcccctgtgagtgtgggcacctgtgcatgcaatgctccgactctgcagaggtgccaagtgcccctgctgggccagtcccagagagttaggaagtcaaggcctgcaactcctggttcttcctgtttggaccagttcttgtgccattggcaggatgagaggcagcagccaggcgggagctgtgtctagcagcacctgtagcccacgtgctgctaattagctggaaaactggcgaaggcagaacctttgctaccaggaatcttgacatgtggggtctgtctttgagaatttgtaaatgaacagtccaatatttcctctcggctcattttgcacatccatttttggggaaatgtgatttctctctctttttttttttttttttttgcctaaggcacaatctcaagaggtcctgagaccacgtacccatgatttttttcttgctctgtgatacccaataactccttcacctaagccctgttgttgattttgaagtgcttcctaggccagtgattttagcttctgccagctgcttttgccagtagattaacgtgtttttatttttcaaactccgtgtttcctaacgtggagtgtatgggtctaagagagcctgctgtcctccctgccttccaccttggagaggaggctggacgcatcagcagtggccagggcaggtcgcaaaatctcccagcctagagaccacacctgaaacggctgaagccagcttgcacaagggctgctgtccctctgcggcaggcagagctggtgggggcaggggtcacagagcagtcatagacaccatggaccagggcaggagaagggcagatggcacatgggcacaacagggccttgtccttagagcactggggggtcatggctgggaggggcatggcaggggctggcatccctgtagagccagaggggccacccagggcagtgacattccagatatgttgggctcacctcatccttgctgtgagactggagttccatggggacatgaagtcagtacaccgcagagctgctcagctgctctacctctcgctgacttttttgttgcacatatacattttctttcaattagcatttatttcagcttttatttaagctttttgacagtacatgtaaaatatatgattataaccatttaaaaataccttatgtacctggttttttttggaaactagatagaaatatatttatcttttacataaaagaagtgtgtagtggggtggtccagggctttgtggtggcttagtggccatcggggtcccaggctcttcccacctttctcttttgggtccacctcttggctcctggcttccttcctctggggcctgactgtccaggatggcagctggagctcctgctctgggcacggttggcatcagagcccactgctccccatccactctttaatccagatgctggtaatgtccctttccaaggcataactaagataagctggaaggttcttacgaggctttgctagaggccctgggaggtggggggaaaggcaagagggcagtgcccacccatagaccgggtcacatgacctggcatcagcgccgtggggtcctttgggcttcacctccctctcccctctgcccccacgtcatcccaccctcatcctcaccctcaccattcccatcccgtaccgtaatgtcccttgcaaggcctaactctgtgaggaatgaaaccacctcatccttgttccaataacatgagtgaccgaatttacaaacgagtgaatgtggacctctggaaacattacccagcttgtttttacttttcactttctctttctgcccctttcatttccgtaggagccctttacctagatgagaagtgtccccccgcctggggaatatatcagtcagaacaatcttcctgcagacatgcaccattagacccgagtgacggtggtgccatttaaacctcagagcaggtaaaaggtggtcctgaaacctgtctacccacagggtgctatggaatctgaatcacttcttttttcctagagccctggggtggggagctccctcaagtgttcacatgtgtgtggaatgaggaacacccatctccttggccctctccaccctgaagagttagttattaaaataattggcaagctcttgcaaatgtcagtcatccattgttcagaatggaatagcaataatacatccctggctgccctgggcttggccaggattactcactgaaggcctcagggttactggcacacactttcttttcctaataatcccatcccctcagctttcctaaggctagagtgaatttcgtgttcctttagtttacataagatggtgaacttggcaaaagctatcattaaacagaagctaagagaaagcctatgtcgtggaatccagaatgggtattgccattcactgctgtccacagaagctgtcttgaatttctttctgtgtcttttctttttttttctttaagactgttgtttaccagactgggctctgtggaacacaggtgtcctgggagatggttaatcattacaaaatattggtaacaatctaaagatgcatacataagagagtggtcaaataaaccattttccattca

SEQ ID NO: 3 - protein, 183 aa, Homo sapiens
MAAGRLFLSRLRAPFSSMAKSPLEGVSSSRGLHAGRGPRRLSIEGNIAVGKSTFVKLLTKTYPEWHVATEPVATWQNIQAAGTQKACTAQSLGNLLDMMYREPARWSYTFQTFSFLSRLKVQLEPFPEKLLQARKPVQIFERLHFEALMNIPVLVLDVNDDFSEEVTKQEDLMREVNTFVKNL

SEQ ID NO: 4 - DNA, 879 bp, Homo sapiens
  source 1..879: mol_type = other DNA
aacggtgcgctggagcgagtgagcagcgatacctagggcggaagtgctctcggcggaagtgatcgctgtgtgaatcgtgggtgggatggccgcgggccgcctctttctaagtcggcttcgagcacccttcagttccatggccaagagcccactcgagggcgtttcctcctccagaggcctgcacgcggggcgcgggccccgaaggctctccatcgaaggcaacattgctgtgggaaagtccacgtttgtgaagttactcacgaaaacttacccagaatggcacgtagctacagaacctgtagcaacatggcagaatatccaggctgctggcacccaaaaagcctgcactgcccaaagtcttggaaacttgctggatatgatgtaccgggagccagcacgatggtcctacacattccagacattttcctttttgagccgcctgaaagtacagctggagcccttccctgagaaactcttacaggccaggaagccagtacagatctttgagaggctccactttgaggctctgatgaacattccagtgctggtgttggatgtcaatgatgatttttctgaggaagtaaccaaacaagaagacctcatgagagaggtaaacacctttgtaaagaatctgtaaccaataccatgaagttcaggctgtgatctgggctccctgactttctgaagctagaaaaatgttgtgtctcccaaccacctttccatccccagcccctctcatccctggagcactctgccgctcaagagctggtttgttaattattgttagactttgccattgttttcttttgtacctgaagcattttgaaaataaagtttacttaagttatgcttgtttttctaaaaaaaaaaaaaaaaaaa

SEQ ID NO: 5 - protein, 423 aa, Homo sapiens
MLLLRLPPHRSHASPLDCKLQDRCRKCYSPRSGQACPPALAAAWLRRCERRGGRPRGGRRKELTLGLRPARCSAPGPAKDDAWRPQAGRSSSDTNESEIKSNEEPLLRKSSRRFVIFPIQYPDIWKMYKQAQASFWTAEEVDLSKDLPHWNKLKADEKYFISHILAFFAASDGIVNENLVERFSQEVQVPEARCFYGFQILIENVHSEMYSLLIDTYIRDPKKREFLFNAIETMPYVKKKADWALRWIADRKSTFGERVVAFAAVEGVFFSGSFAAIFWLKKRGLMPGLTFSNELISRDEGLHCDFACLMFQYLVNKPSEERVREIIVDAVKIEQEFLTEALPVGLIGMNCILMKQYIEFVADRLLVELGFSKVFQAENPFDFMENISLEGKTNFFEKRVSEYQRFAVMAETTDNVFTLDADF

SEQ ID NO: 6 - DNA, 4913 bp, Homo sapiens
  source 1..4913: mol_type = other DNA
atgaggtaaatgttgctgttgcgtcttccccctcaccgcagtcacgccagcccgttagattgcaagttgcaggaccgctgtaggaaatgttattcgccgcggtcaggacaggcctgtccgcccgccctcgccgcagcctggcttcgtcgttgcgagcgccggggaggccgtccccggggagggcggaggaaggagctgactttgggtttgcgtcccgctcgctgctctgccccggggccagccaaggacgacgcttggaggcctcaggccgggagatcatcttcagacaccaacgaaagtgaaataaagtcaaatgaagagccactcctaagaaagagttctcgccggtttgtcatctttccaatccagtaccctgatatttggaaaatgtataaacaggcacaggcttccttctggacagcagaagaggtcgacttatcaaaggatctccctcactggaacaagcttaaagcagatgagaagtacttcatctctcacatcttagccttttttgcagccagtgatggaattgtaaatgaaaatttggtggagcgctttagtcaggaggtgcaggttccagaggctcgctgtttctatggctttcaaattctcatcgagaatgttcactcagagatgtacagtttgctgatagacacttacatcagagatcccaagaaaagggaatttttatttaatgcaattgaaaccatgccctatgttaagaaaaaagcagattgggccttgcgatggatagcagatagaaaatctacttttggggaaagagtggtggcctttgctgctgtagaaggagttttcttctcaggatcttttgctgctatattctggctaaagaagagaggtcttatgccaggactcactttttccaatgaactcatcagcagagatgaaggacttcactgtgactttgcttgcctgatgttccaatacttagtaaataagccttcagaagaaagggtcagggagatcattgttgatgctgtcaaaattgagcaggagtttttaacagaagccttgccagttggcctcattggaatgaattgcattttgatgaaacagtacattgagtttgtagctgacagattacttgtggaacttggattctcaaaggtttttcaggcagaaaatccttttgattttatggaaaacatttctttagaaggaaaaacaaatttctttgagaaacgagtttcagagtatcagcgttttgcagttatggcagaaaccacagataacgtcttcaccttggatgcagatttttaaaaaacctctcgttttaaaactctataaacttgtcattggtaaatagtagtctattttcctctgcttaaaaaaaattttaagtatatcctttaaaggactgggggtttgctcaaaaggaaatccaaaacctattctaaacaatttgcatttatataattttcctgtttaacaacaagagtgtgacctaaatgcttttgtcttgtcactgaaataaaagatggcattatgtggttaagagcatggggcgaggggtcagacatgagtctaaggttctgcccttactccagtgtgtgacccttggcaagtcagttaatcttggtaaacctcggtgtacttatctttaaaatgggagtaatagtaggtcctaaattcatagagtggatattaggattaggatgcaaaaataaatgcttaaccaacactactactgttagcaccactactaattatcattcattgataatattaattgcaatgatgttgtaataaaatactctcatttccttaaaataattgtgattctaggtcctaggatctagaattagatctttgtatttttaatgcttaggggaagaatataagtatctccttaaaaagaacataattctcattcacgcaagaataagttctttgaattccttagtatgtagtgaagaaaatttagttgttagttgctttgggaagcctacttatggagtggaaaccaggaggttatcatggtagttgaccttataagaaaaatgattcttcttcagaaattaaaaacataactattgccagatttagctctggaatgtttagaatcaggctagaatagcattttccaaagaatattctaagagctattagctcctctagatatttttttgggggaaaaaggggattctgtggtcagatgagtttgggaaatgctgaacacttcattcttctttagcaagtacagtcagtacatcaaagactgagcagttcagtggtacataaatttatctcgccctgcatattcccaacatacttaacacagatgttttttacctgttaacatctcacccagctagtgttcctcagaacaaagattggaaaaagctggccgagaaccatttatacatagaggaagggcttatggactgagaaagggagaacatggtagggattattgaatcatttcaaatttataccagcctgaatagtgtaccagcaattgacttaggctgtgtttctttatggttttaaaactcttgagctgttataagagatagttcttttaatgtgactatgcaacatgatagccaatggtgagggaaaaggaggtttctctagaagagtctgatgaaaggccgggaaccaaggtttttgagaagtctgcccctatttatttttagtaagtatcaagaggtagcctgagcctagttagagttagacctgtctttggatgaagaagtcttaatactgaaatactgaatttttaatacattattatttggtattctgtataccccttcaagcagttgtttcccattcccaacaaactgtactttatacaattctggatgctaaaacttagagattttctctttgcataaattttggctccattctttccataacaatctaatcaaaactgggagttctcaagtgaatgcaaaaggagcaggccataactttatttgttagatacactgtcagaaacttgagatcttttggcctatgataataccattaatttttgcattgcttcagtttgccaagtgtttttacatcatctcatttgatctcaaaacagcttgacagagcaactgttattgaaatattacagatggaaagaatgaggctcagggaagttaaatgacttggccaagatctgctcatcgtcactgtctgtacagtatttttttttagaggttgtaatgtctcagatttagtcctttaccatctatgttgatttgcttttgtctatttcctcattaattgaatatactttaaatatatatattaaagtatcaaaatatagagagacatttgaactgtattcaggtaatatgtttaaagatatttatatattgccatacaaaaacttaacatttaaaactgataatatctgtaatgacatcagaatgaaagaaaaaaaattgtacagtgtatattcctttgttttgaatccaaatctttttcataggtaatgacagatgccttaatgtgaagcttatttataatagcaataaacctaactggatttggatgaagaagtcttaatactgacatactggatttttaatgcactggtttgttatttggtattctatctctttttccaggcctccaggttgcacatttatttattatgttcaatactttggttcttagttcttaaagaatcaagaagttgtgtaatcttttaaaaatattatcttgcagataaagaaaaaaattaagagtgtgtttacaactgttttctcttttttacagtacatgtatttaaatcattgctataataaagttaagttcattaggaatataaaaacttgcagttctatgatagattgcatttattaaaaatgtttcattgtatcacatagaaatatggccaggaaggacttgagaagacagtttgatccattgcttttagacaggactgggttttgctgtccaattatatacaataatagtttttcttacaactaagctggccccagccttgtcttgatattaatacatgaaatttttataattgtctcattgtctcatttagaaacatccatatttttctgctttttctattgccattttttatttgtgcatgaattgattattgagaaaatgtagcagtttgcatatttaaaaattaatcattttgcattttacatttaaatatgctaacatcactgtcatagaattcccaaatttcatttgtagatactgaactaagggctaatgtcaggagctgatttttaatgataaagctgcagatgggctaaataaaagccaaattaatcctacaatcaggtattatgtttttaaaccaagttgagtgaattggtagtggacttgggaaatcttccccagcagaatctggatgaatggcacagaattgaaatctctttgtttcccaccatttccctttaagtgctctgctcctttgtaaaaagttaaagatttgaaagagaatctcatattcccgaggcattaggaagaaaggatttaatcccttcaatttggggcttaatcttgtttaaaaaaatgtaagtgaagatggaaggctggagagaatgattgctttttgtacagttaaataaggtcacaatattcttacatactttgttttacaactgtgttttcattttttcaaatgtctggccatttagcaaagttatttactatttactgtgtacatagaaagctttattatgtgtggtgtatctaaattttttttgctgaaatacattatggtcaatcaagccaagcctgcatgtacagaatttgtttttttttcaaataaattagttgttttcttatttttttggcttagtatgttgaaataaactatggtatcttcatcattttgtacatttcctttttgaggaaggtttctttataagtgcaagggctaccctaataaaggaatgtatatacttact

SEQ ID NO: 7 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 8 - DNA, 1679 bp, Homo sapiens
  source 1..1679: mol_type = other DNA
cgactgccgagctccgccctccaggcggccccacccgcctgccgtcctggggcgccgccgccccgccgccggcagtggaccgctgtgcgcgaaccctgaaccctacggtcccgacccgcgggcgaggccgggtacctgggctgggatccggagcaagcgggcgagggcagcgccctaagcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaaaaaaaaaa

SEQ ID NO: 9 - protein, 463 aa, Homo sapiens
MAASMFYGRLVAVATLRNHRPRTAQRAAAQVLGSSGLFNNHGLQVQQQQQRNLSLHEYMSMELLQEAGVSVPKGYVAKSPDEAYAIAKKLGSKDVVIKAQVLAGGRGKGTFESGLKGGVKIVFSPEEAKAVSSQMIGKKLFTKQTGEKGRICNQVLVCERKYPRREYYFAITMERSFQGPVLIGSSHGGVNIEDVAAESPEAIIKEPIDIEEGIKKEQALQLAQKMGFPPNIVESAAENMVKLYSLFLKYDATMIEINPMVEDSDGAVLCMDAKINFDSNSAYRQKKIFDLQDWTQEDERDKDAAKANLNYIGLDGNIGCLVNGAGLAMATMDIIKLHGGTPANFLDVGGGATVHQVTEAFKLITSDKKVLAILVNIFGGIMRCDVIAQGIVMAVKDLEIKIPVVVRLQGTRVDDAKALIADSGLKILACDDLDEAARMVVKLSEIVTLAKQAHVDVKFQLPI

SEQ ID NO: 10 - DNA, 2182 bp, Homo sapiens
  source 1..2182: mol_type = other DNA
ccgcctgtcgcctgtgcgcctgcgcgcggcgccgaggggacggggtccgactcagaaatggcggcctccatgttctacggcaggctagtggccgtggccacccttcggaaccaccggcctcggacggcccagcgggctgctgctcaggttctgggaagttctggattgtttaataaccatggactccaagtacagcagcaacagcaaaggaatctctcactacatgaatacatgagtatggaattattgcaagaagctggtgtctccgttcccaaaggatatgtggcaaagtcaccagatgaagcttatgcaattgccaaaaaattaggttcaaaagatgtcgtgataaaggcacaggttttagctggtggtagaggaaaaggaacatttgaaagtggcctcaaaggaggagtgaagatagttttctctccagaagaagcaaaagctgtttcttcacaaatgattgggaaaaaattgtttaccaagcaaacgggagaaaagggcagaatatgcaatcaagtattggtctgtgagcgaaaatatcccaggagagaatactactttgcaataacaatggaaaggtcatttcaaggtcctgtattaataggaagttcacatggtggtgtcaacattgaagatgttgctgctgagtctcctgaagcaataattaaagaacctattgatattgaagaaggcatcaaaaaggaacaagctctccagcttgcacagaagatgggatttccacctaatattgtggaatcagcagcagaaaacatggtcaagctttacagcctttttctgaaatacgatgcaaccatgatagaaataaatccaatggtggaagattcagatggagctgtattgtgtatggatgcaaagatcaattttgactctaattcagcctatcgccaaaagaaaatctttgatctacaggactggacccaggaagatgaaagggacaaagatgctgctaaggcaaatctcaactacattggcctcgatggaaatataggctgcctagtaaatggtgctggtttggctatggccacaatggatataataaaacttcatggagggactccagccaacttccttgatgttggtggtggtgctacagtccatcaagtaacagaagcatttaagcttatcacttcagataaaaaggtactggctattctggtcaacatttttggaggaatcatgcgctgtgatgttattgcacagggtatagtcatggcagtaaaagacttggaaattaaaatacctgttgtggtacggttacaaggtacacgagtcgatgatgctaaggcactgatagcggacagtggacttaaaatacttgcttgtgatgacttggatgaagctgctagaatggttgtaaagctctctgaaatagtgaccttagcgaagcaagcacatgtggatgtgaaatttcagttgccaatatgatctgaaaacccagtggatggctgaaggtgttaaatgtgctataatcattaagaatactgtgttctgtgttattgttctttttctttttagtgtgtggagattgtaattgccatctaggcacacaaacatttaaaaggatttggactgcatttaattgtaccattcagaatggactgtttgtacgaagcatgtataatgcagttatcttctttcttttgtcgcagccagtcttttttgcttctcctacaaaacgtaacttgcaatttgccagtttattattgttggatacaaagttcttcattgataagagtcctataaataagataaatacgaagataaagctttattctttagtgttaaaatacagtatatctaataactagcctcattagtagagcagtatattaaaacaatgttttatgtaaaaagtgtttatcttcagcaccaaatacatgataaatgtatcaatcactatttataaacagagctttcaaacactcctcagaatattcttctaagtattttgatgaagtaactttgtaattatttgaacattgttttaatcattaggaaacactgattaactgcaagtcttcatgattctgtcatattaagaaacacctgtaggtttgcttcaaataaaggcatatataccaaggacttacagacaaaattaagaatgtcaatttaagttaataaaaatctcccaatatgaaaaaaaaaaaaaaaaaaa

SEQ ID NO: 11 - protein, 346 aa, Homo sapiens
MTATLAAAADIATMVSGSSGLAAARLLSRSFLLPQNGIRHCSYTASRQHLYVDKNTKIICQGFTGKQGTFHSQQALEYGTKLVGGTTPGKGGQTHLGLPVFNTVKEAKEQTGATASVIYVPPPFAAAAINEAIEAEIPLVVCITEGIPQQDMVRVKHKLLRQEKTRLIGPNCPGVINPGECKIGIMPGHIHKKGRIGIVSRSGTLTYEAVHQTTQVGLGQSLCVGIGGDPFNGTDFIDCLEIFLNDSATEGIILIGEIGGNAEENAAEFLKQHNSGPNSKPVVSFIAGLTAPPGRRMGHAGAIIAGGKGGAKEKISALQSAGVVVSMSPAQLGTTIYKEFEKRKML

SEQ ID NO: 12 - DNA, 1270 bp, Homo sapiens
  source 1..1270: mol_type = other DNA
gtcattggcgtatgaccgcaacccttgccgctgccgctgacatcgctaccatggtctccggcagcagcggcctcgccgccgcccgtctcctgtcgcgcagcttcctcctgccgcagaatggaattcggcattgttcctacacagcttctcggcaacatctctatgttgataaaaatacaaagattatttgccagggtttcactggcaaacagggcacctttcacagccagcaggcattggaatatggcaccaaactcgttggaggaaccactccagggaaaggaggccagacacatctgggcttacctgtctttaatactgtgaaggaggccaaagaacagacaggagcaacggcttctgtcatttatgttcctccgccttttgctgctgctgccattaatgaagctattgaggcagaaattcccttggttgtgtgtatcactgaaggaattccccagcaggacatggtacgagtcaagcacaaactgctgcgccaggaaaagacaaggctaattgggcccaactgccctggagtcatcaatcctggagaatgtaaaattggcatcatgcctggccatattcacaaaaaaggaaggattggcattgtgtccagatctggcaccctgacttatgaagcagttcaccaaacaacgcaagttggattggggcagtctttgtgcgttggcattggaggtgatccttttaatggaacagattttattgactgcctcgaaatctttttgaacgattctgccacagaaggcatcatattgattggtgaaattggtggtaatgcagaagagaatgctgcagaatttttgaagcaacataattcaggtccaaattccaagcctgtagtgtccttcattgctggtttaactgctcctcctgggagaagaatgggtcatgccggggcaattattgctggaggaaaaggtggagctaaagagaagatctctgcccttcagagtgcaggagttgtggtcagtatgtctcctgcacagctgggaaccacgatctacaaggaatttgaaaagaggaagatgctatgaaagaaaaaaaaaattcctaaaactgtggaatggatcacgtagacatgtaacccagcagcagtttgcttctgttgtccactgattaatcagcctatgtgcctgacactggtcttgcagtacaactggaagccaaaacaaggtggaagatgtcctgaattaagatgttttcaccacattgtattacagagacagccaataaatctactatttgatttcaa

SEQ ID NO: 13 - protein, 176 aa, Homo sapiens
MALWRAYQRALAAHPWKVQVLTAGSLMGLGDIISQQLVERRGLQEHQRGRTLTMVSLGCGFVGPVVGGWYKVLDRFIPGTTKVDALKKMLLDQGGFAPCFLGCFLPLVGALNGLSAQDNWAKLQRDYPDALITNYYLWPAVQLANFYLVPLHYRLAVVQCVAVIWNSYLSWKAHRL

SEQ ID NO: 14 - DNA, 1002 bp, Homo sapiens
  source 1..1002: mol_type = other DNA
agttcctaggccagcctgtcacgtgggagggaggctcggcgctcaggaagcatggcactctggcgggcataccagcgggccctggccgctcacccgtggaaagtacaggtcctgacagctgggtccctgatgggcctgggtgacattatctcacagcagctggtggagaggcggggtctgcaggaacaccagagaggccggactctgaccatggtgtccctgggctgtggctttgtgggccctgtggtaggaggctggtacaaggttttggatcggttcatccctggcaccaccaaagtggatgcactgaagaagatgttgttggatcaggggggctttgccccgtgttttctaggctgctttctcccactggtaggggcacttaatggactgtcagcccaggacaactgggccaaactacagcgggattatcctgatgcccttatcaccaactactatctatggcctgctgtgcagttagccaacttctacctggtcccccttcattacaggttggccgttgtccaatgtgttgctgttatctggaactcctacctgtcctggaaggcacatcggctctaagcctgcctcactccatcgtttccaccttgcagtgatgcagcttgaccctggaacggtcagacaacctcctcaaagtgggcataccagtttccacggggttgggttgccggtcagagcttaagaggactagcaccctgcaatgcccctcttcactctaaaatgtacactgactgctttagagcccttgataatagtcttattcccaccacatactaggcactccataaatatctgttgaaccttcatgaccttatcaactttacacccatatcccagcaaatgccactcatccccactcttcatagacacatttgttactctaaccctgcctaggcttcttgtagctccagctctttagagactcccggaaccctttatatggtgcctcagtaaatatgttattaaatatgtaatccggaa

SEQ ID NO: 15 - protein, 1239 aa, Homo sapiens
MSRLLWRKVAGATVGPGPVPAPGRWVSSSVPASDPSDGQRRRQQQQQQQQQQQQQPQQPQVLSSEGGQLRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPTGASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKEDPWLWDLEWDLQEFKQKKAKKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 16 - DNA, 4452 bp, Homo sapiens
  source 1..4452: mol_type = other DNA
gcggaccggccgggtggaggccacacgctaccccgaggctgcgtaggccgcgcgaagggggacgccgtgccgtgggcctggggtcgggggagcagcagaccgggaagcaccgatttggggtggaaggcaggcatggtcaaacccatttcactgacaggagagcagagacaggacgtgtctctctccacgtcttccagccagtaaaagaagccaagctggagcccaaagccaggtgttctgactcccagcgtgggggtccctgcaccaaccatgagccgcctgctctggaggaaggtggccggcgccaccgtcgggccagggccggttccagctccggggcgctgggtctccagctccgtccccgcgtccgaccccagcgacgggcagcggcggcggcagcagcagcagcagcagcagcagcagcagcaacagcagcctcagcagccgcaagtgctatcctcggagggcgggcagctgcggcacaacccattggacatccagatgctctcgagagggctgcacgagcaaatcttcgggcaaggaggggagatgcctggcgaggccgcggtgcgccgcagcgtcgagcacctgcagaagcacgggctctgggggcagccagccgtgcccttgcccgacgtggagctgcgcctgccgcccctctacggggacaacctggaccagcacttccgcctcctggcccagaagcagagcctgccctacctggaggcggccaacttgctgttgcaggcccagctgcccccgaagcccccggcttgggcctgggcggagggctggacccggtacggccccgagggggaggccgtacccgtggccatccccgaggagcgggccctggtgttcgacgtggaggtctgcttggcagagggaacttgccccacattggcggtggccatatccccctcggcctggtattcctggtgcagccagcggctggtggaagagcgttactcttggaccagccagctgtcgccggctgacctcatccccctggaggtccctactggtgccagcagccccacccagagagactggcaggagcagttagtggtggggcacaatgtttcctttgaccgagctcatatcagggagcagtacctgatccagggttcccgcatgcgtttcctggacaccatgagcatgcacatggccatctcagggctaagcagcttccagcgcagtctgtggatagcagccaagcagggcaaacacaaggtccagccccccacaaagcaaggccagaagtcccagaggaaagccagaagaggcccagcgatctcatcctgggactggctggacatcagcagtgtcaacagtctggcagaggtgcacagactttatgtaggggggcctcccttagagaaggagcctcgagaactgtttgtgaagggcaccatgaaggacattcgtgagaacttccaggacctgatgcagtactgtgcccaggacgtgtgggccacccatgaggttttccagcagcagctaccgctcttcttggagaggtgtccccacccagtgactctggccggcatgctggagatgggtgtctcctacctgcctgtcaaccagaactgggagcgttacctggcagaggcacagggcacttatgaggagctccagcgggagatgaagaagtcgttgatggatctggccaatgatgcctgccagctgctctcaggagagaggtacaaagaagacccctggctctgggacctggagtgggacctgcaagaatttaagcagaagaaagctaagaaggtgaagaaggaaccagccacagccagcaagttgcccatcgagggggctggggcccctggtgatcccatggatcaggaagacctcggcccctgcagtgaggaggaggagtttcaacaagatgtcatggcccgcgcctgcttgcagaagctgaaggggaccacagagctcctgcccaagcggccccagcaccttcctggacaccctggatggtaccggaagctctgcccccggctagacgaccctgcatggaccccgggccccagcctcctcagcctgcagatgcgggtcacacctaaactcatggcacttacctgggatggcttccctctgcactactcagagcgtcatggctggggctacttggtgcctgggcggcgggacaacctggccaagctgccgacaggtaccaccctggagtcagctggggtggtctgcccctacagagccatcgagtccctgtacaggaagcactgtctcgaacaggggaagcagcagctgatgccccaggaggccggcctggcggaggagttcctgctcactgacaatagtgccatatggcaaacggtagaagaactggattacttagaagtggaggctgaggccaagatggagaacttgcgagctgcagtgccaggtcaacccctagctctgactgcccgtggtggccccaaggacacccagcccagctatcaccatggcaatggaccttacaacgacgtggacatccctggctgctggtttttcaagctgcctcacaaggatggtaatagctgtaatgtgggaagcccctttgccaaggacttcctgcccaagatggaggatggcaccctgcaggctggcccaggaggtgccagtgggccccgtgctctggaaatcaacaaaatgatttctttctggaggaacgcccataaacgtatcagctcccagatggtggtgtggctgcccaggtcagctctgccccgtgctgtgatcaggcaccccgactatgatgaggaaggcctctatggggccatcctgccccaagtggtgactgccggcaccatcactcgccgggctgtggagcccacatggctcaccgccagcaatgcccggcctgaccgagtaggcagtgagttgaaagccatggtgcaggccccacctggctacacccttgtgggtgctgatgtggactcccaagagctgtggattgcagctgtgcttggagacgcccactttgccggcatgcatggctgcacagcctttgggtggatgacactgcagggcaggaagagcaggggcactgatctacacagtaagacagccactactgtgggcatcagccgtgagcatgccaaaatcttcaactacggccgcatctatggtgctgggcagccctttgctgagcgcttactaatgcagtttaaccaccggctcacacagcaggaggcagctgagaaggcccagcagatgtacgctgccaccaagggcctccgctggtatcggctgtcggatgagggcgagtggctggtgagggagttgaacctcccagtggacaggactgagggtggctggatttccctgcaggatctgcgcaaggtccagagagaaactgcaaggaagtcacagtggaagaagtgggaggtggttgctgaacgggcatggaaggggggcacagagtcagaaatgttcaataagcttgagagcattgctacgtctgacataccacgtaccccggtgctgggctgctgcatcagccgagccctggagccctcggctgtccaggaagagtttatgaccagccgtgtgaattgggtggtacagagctctgctgttgactacttacacctcatgcttgtggccatgaagtggctgtttgaagagtttgccatagatgggcgcttctgcatcagcatccatgacgaggttcgctacctggtgcgggaggaggaccgctaccgcgctgccctggccttgcagatcaccaacctcttgaccaggtgcatgtttgcctacaagctgggtctgaatgacttgccccagtcagtcgcctttttcagtgcagtcgatattgaccggtgcctcaggaaggaagtgaccatggattgtaaaaccccttccaacccaactgggatggaaaggagatacgggattccccagggtgaagcgctggatatttaccagataattgaactcaccaaaggctccttggaaaaacgaagccagcctggaccatagcactgcctggaggctctgtatttgctcccgtggagcttcatcggggtggtgcaggctcccaaactcaggctttcagctgtgctttttgcaaaagggcttgcctaaggccagccatttttcagtagcaggacctgccaagaagattccttctaactgaaggtgcagttgaattcagtgggttcagaaccaagatgccaacatcggtgtggactacaggacaaggggcattgttgcttgttgggtaaaaatgaagcagaagccccaaagttcacattaactcaggcatttcatttattttttccttttcttcttggctggttctttgttctgtcccccatgctctgatgcagtgccctagaaggggaaagaattaatgctctaacgtgataaacctgctccaaggcagtggaaataaaaagaaggaaaaaaaagactctatcttctcaaaaaaaaaaaaaaaaa

SEQ ID NO: 17 - DNA, 705 bp, Homo sapiens
  source 1..705: mol_type = other DNA
atgggagcattttgccagcgacctagttccgataaagaacaggagaaagagaagaagagcgtcatctgtgtggaaggtaacatcgctagtggcaaaacgacctgtttggaatttttctctaatgccaccgatgtggaggtgcttacagagcctgttagtaaatggcgaaatgttcgaggacataatccgctgggactgatgtatcacgatgcctccagatggggactgacccttcagacttatgtccaacttacaatgcttgacaggcacactagaccgcaggttagcagcgtgaggctgatggagcgctcaattcactctgccaggtatatattcgttgagaacctctaccgatctggtaagatgcctgaggtagattacgtagtgctgagtgaatggtttgactggattcttcgcaacatggatgtatcagtggatctcattgtttaccttaggactaacccggagacgtgttatcagcgcttgaaaaagcgctgccgggaagaggaaaaagtgattcctctggaatacttggaagctattcatcaccttcacgaagagtggctgataaaaggttccctgtttcccatggcggcccccgtgcttgtcatagaagcggatcaccacatggaaaggatgctcgaacttttcgagcaaaatcgagaccggattctcacgccggagaatcgaaaacattgcccatag

SEQ ID NO: 18 - DNA, 552 bp, Homo sapiens
  source 1..552: mol_type = other DNA
atggctgcagggaggctttttttgtctagattgagggcaccttttagctccatggcgaagtctccgcttgagggggtgagtagcagccgggggctccatgctggcagagggccccggaggctgtctattgaggggaatattgcagttggtaagagcacgtttgttaaattgctcacgaagacctatccagaatggcacgttgctacagaaccagtagctacatggcaaaacattcaagcagcaggcacacagaaggcttgcactgcacagtctctcggaaacctgctcgatatgatgtacagagagcccgcccggtggtcatacacgtttcagaccttcagctttctttctcggttgaaggtacagttggagcccttcccagagaagttgcttcaagcacggaaacctgtacagatattcgaacgattgcatttcgaggcgctgatgaacatacctgtgcttgttctggatgtgaacgacgatttttccgaggaagttactaagcaagaggacctcatgcgggaggtaaacacgttcgttaagaacctgtaa

SEQ ID NO: 19 - DNA, 1449 bp, Homo sapiens
  source 1..1449: mol_type = other DNA
atggcagcattgatgacaccaggaacgggggctccgcctgcgcctggcgatttttcaggagaggggagtcaaggtctgccggacccttcaccggaacccaaacagcttcccgagcttattagaatgaagcgagatgggggtcgcctctctgaggctgacatacgcggctttgttgcggcggtcgtcaacggctcagcacaaggtgcgcaaataggagcgatgctgatggctatcaggctgaggggaatggatttggaagagaccagtgtgctgactcaagcgctggcacagagcggccagcagcttgaatggccagaagcttggcgccaacaattggtcgataaacactcaaccgggggcgtcggggacaaagtctcccttgtactggcacctgctctggctgcgtgcggatgtaaggttcctatgattagcgggcggggcttgggacatacgggaggaacgttggacaaactcgaatccatccctggtttcaacgtgattcagagccctgaacaaatgcaagtactcctggatcaggcaggctgctgtattgttggccaaagcgaacaactcgtgcctgcggatggtatcctctatgctgccagggatgtgaccgccacagttgactccctcccgctgataacagcgtcaattttgagtaagaagctcgtcgaagggctcagcgctcttgtggttgatgtaaaatttgggggggccgcagtattccccaaccaggaacaagcacgggaactggcaaaaaccctggttggtgtgggcgcttcactgggcctgagagttgccgctgccttgaccgccatggataagccgctcggtaggtgcgtaggtcatgcacttgaagtagaagaagcccttttgtgcatggatggggccggtccccctgacttgcgggatctcgtaaccacgcttggcggcgcgttgctttggctttctggacacgctggtacccaagcgcaaggggcagcaagagtcgcagcagcgcttgatgacggatcagctcttgggcgatttgaaagaatgcttgcagctcagggcgtagatccagggctggcgcgggccctttgctcaggttctccagcagagcgccgacagctccttcccagggcgcgagagcaggaagaattgctggccccggctgatggtaccgtcgaactcgtacgggctttgccgctggctcttgttttgcacgaactgggggctgggcggagtcgcgccggtgaacctctcagactcggtgtgggtgcggagctcctcgttgacgtcgggcaacgacttcgcaggggaaccccttggcttagggtacacagggacgggccagcactcagcggccctcagtccagggcccttcaagaagctctcgtgctgagtgatcgagcgccttttgccgctccctcaccatttgctgaattggtattgccaccccagcagtaa

SEQ ID NO: 20 - DNA, 1272 bp, Homo sapiens
  source 1..1272: mol_type = other DNA
atgctgcttctcagactgccaccccaccgcagtcatgcttccccacttgactgtaaacttcaagataggtgccggaagtgttactcaccccgatcaggacaagcgtgccctcctgcgttggcggccgcctggcttcggcggtgtgaacgccgaggaggtcggccgaggggtgggcgacgcaaggaactgacccttggcctgcgccctgcacggtgttcagctcccggtcctgccaaggatgatgcatggagacctcaggcaggtcgctcctcctcagataccaacgagagtgaaattaaatctaatgaagaaccccttcttcgcaagagtagtcgcaggttcgtgatattccccatacaatatcctgatatctggaaaatgtacaagcaagcccaagcatcattctggaccgcagaagaggttgatctcagtaaggacctcccacactggaacaaactgaaagccgacgaaaaatatttcatatcccatatacttgctttctttgcagcctcagatggaattgtgaatgaaaacttggtagagcgcttctctcaagaggtccaagttcctgaagcacgatgcttttacggatttcaaatcctgatagaaaatgtacattccgaaatgtattccttgcttatagacacgtatatcagagatcccaaaaaaagggagttcttgttcaatgcgatcgagaccatgccatatgttaagaaaaaagccgattgggccctgaggtggatagctgataggaagtctacatttggggaacgcgttgttgccttcgccgccgtcgagggagtcttcttctccggatccttcgctgctatcttttggctgaaaaaacgcggcttgatgcctggtttgacctttagcaatgagttgatatcacgggatgaagggctgcattgtgactttgcctgccttatgttccagtacttggtgaacaaaccgtctgaggaacgggtaagggaaatcatagtggatgcagtaaaaatagagcaggagttccttactgaagcgctccccgtagggctcattggcatgaactgcattcttatgaagcagtacatcgagttcgtggccgatagacttttggtggagctcgggttctcaaaggtttttcaggctgaaaatccttttgattttatggagaacatatctctggagggcaagactaacttttttgaaaaaagagtatccgagtatcaaaggtttgccgtgatggcagaaacaacagataatgtcttcacacttgatgcggatttttaa

SEQ ID NO: 21 - DNA, 1392 bp, Homo sapiens
  source 1..1392: mol_type = other DNA
atggcggcttctatgttctatggacgcctggtcgccgttgccacgttgcggaatcacaggccccgaactgcacaaagggctgccgctcaggtgcttggaagcagcggattgttcaacaatcacggcctccaagttcagcaacagcagcagagaaacctctctctgcatgagtatatgagtatggaactgctccaggaagcaggggtgtctgtaccaaagggctacgtagcaaaaagccccgacgaggcgtacgccatagctaaaaaactgggaagtaaggacgtagttatcaaagcacaggttctggccggtggccgcggtaagggaaccttcgagagtggtttgaaaggcggtgtcaagatcgtatttagtcccgaggaggccaaggctgtaagttcccagatgatcggcaaaaaattgttcacaaagcaaaccggcgaaaagggtagaatttgcaaccaagtacttgtctgcgaaagaaagtatccgagaagggagtattatttcgctataaccatggaaagatcattccaaggtcccgtgctcatcggcagttcacacgggggcgttaatatagaggatgtcgctgcagagtctccggaggccataattaaggagcctatagacatcgaggaaggcattaaaaaagaacaagccttgcaactggcacaaaaaatgggttttcctccgaacatcgtcgagagtgccgcagaaaatatggtgaaactgtacagcctgtttttgaagtacgatgcgacaatgatagaaattaatccgatggtcgaggactcagatggagccgtgctttgtatggacgccaaaattaacttcgattcaaacagcgcttatcgacagaagaaaattttcgatttgcaagattggacccaagaggatgagcgagacaaagatgccgccaaagcaaatctcaattatataggactcgatggtaatattggatgtttggtcaatggtgccggtctcgcgatggcaactatggatataatcaagttgcatggtgggactcccgccaactttcttgacgtcgggggaggcgccaccgttcaccaagttacggaggctttcaaactcataacctctgacaagaaggtgctggcgattcttgtaaatatctttggtggcattatgcggtgtgatgttattgcccagggaatagtaatggctgtcaaagatttggagattaaaatacccgtcgtggtgcggctccaaggtactcgagtagatgatgctaaggctctgattgccgacagcgggctgaagattctggcatgtgatgatttggacgaggcagcgagaatggtcgtgaagctctcagaaatagtaactttggcgaaacaggctcatgtcgatgtaaagttccaacttcctatatga

SEQ ID NO: 22 - DNA, 1041 bp, Homo sapiens
  source 1..1041: mol_type = other DNA
atgaccgctacgcttgcggctgcagccgacatcgcgacaatggtctctggaagttcaggcttggccgcggctcggcttctgagtagaagtttcttgttgccgcaaaacggtatcaggcactgctcctacaccgccagtagacaacacctttatgttgataagaacacgaagataatttgccaaggattcaccggtaaacaggggaccttccattcccagcaggcactggaatatggaactaaattggtaggcggcactacgcctgggaagggtggccaaacacatcttggtcttcccgtttttaacacagtgaaggaggctaaagaacaaacgggggcaacggctagcgttatctatgtcccacccccgtttgccgctgcagccataaatgaggcgattgaagccgagatcccgcttgtagtctgcataacggagggaattccgcaacaagacatggtgcgagtaaagcacaagcttcttcgacaggaaaaaacaagactgataggtccgaattgtcctggcgtaattaaccccggtgaatgcaaaatcggaattatgccgggacatattcacaaaaaaggccgaatcgggatagtcagcagatcaggcaccttgacatacgaagcggttcaccagactacgcaagtcggtttgggacaaagcctttgcgtcggtatcggtggcgacccatttaacgggacggatttcattgactgtctggagatcttcctcaacgattccgcgacagaaggaatcattttgataggagaaataggcgggaacgccgaagagaatgcagcggaattcctcaaacaacataactctggccctaatagtaagccggtggtatcattcatagccggtcttacagcgccgccgggtcgcagaatgggccacgcaggtgcaattatagcggggggcaagggaggtgccaaagaaaagatatccgctcttcaaagcgcaggtgtagtagttagtatgagtccagctcaactgggcacaaccatctacaaagaatttgaaaagcggaaaatgctttga

SEQ ID NO: 23 - DNA, 531 bp, Homo sapiens
  source 1..531: mol_type = other DNA
atggccctctggagagcttaccaacgagccttggccgcgcacccttggaaggtacaggtcttgaccgccggatctttgatgggtcttggagatattatttctcaacagttggttgaacggcgaggcctccaggaacaccaaagggggagaacccttacaatggtaagtctgggttgcggattcgtcgggcctgtcgtggggggatggtataaggttctggatcgctttataccggggaccaccaaggtcgatgccttgaagaagatgttgctggatcagggaggattcgctccgtgttttttgggatgttttctgccacttgttggggctctgaacggactgtccgcgcaggataactgggcgaagttgcaacgcgactacccagacgccctgataacaaattactatctctggccagcagttcagttggccaatttttacctggtacctctccactatcgccttgctgtagtacagtgtgtcgccgtcatctggaactcatacctttcatggaaggctcatagattgtaa

SEQ ID NO: 24 - DNA, 3720 bp, Homo sapiens
  source 1..3720: mol_type = other DNA
atgtccaggctgctctggcggaaggtcgcaggcgccactgtcggtccaggaccagttccagctcccggtcgctgggtgagcagcagcgtgccagctagcgatccgagcgacggtcagcggcgacggcaacaacagcagcaacagcaacaacagcaacaacaacagccccagcagcctcaggttctcagttccgagggtggccaactccgacacaacccactggatatacagatgctctcccgcggtctccacgaacaaatattcggacaagggggtgagatgccgggagaggctgcggtcaggcgcagtgtagaacatctccagaaacacgggttgtggggccaaccggccgttcctctccccgatgttgaactgcggcttccacctctctacggtgataatctggaccagcactttagactgctcgctcaaaagcagagtctcccttacctggaagccgctaacctcctgctccaagcccaattgccccctaaaccgccagcctgggcttgggcggagggatggacgaggtatggacccgaaggggaggctgtgccagttgctataccagaggaacgcgctctggttttcgacgtagaggtttgtctcgcggagggaacttgtcctacactggctgtagcaatttccccttcagcctggtacagctggtgctctcagagattggtggaagaaaggtatagctggactagccagctgagtcccgcggacctcattccacttgaggtacccaccggggcgtcaagcccaactcagagggactggcaggaacaattggtagttgggcataatgtgagttttgacagggctcatatccgcgaacagtatcttatccagggctctagaatgcgattccttgacacgatgagcatgcacatggcaatcagcggacttagttcctttcagaggtcattgtggattgcagccaagcagggaaagcataaggtccaacccccgacaaaacaaggtcagaaatcccagagaaaagcccggcgaggccccgccatcagttcctgggattggttggatatcagtagtgtgaatagccttgctgaggtgcatcgcctgtatgtgggtggacccccacttgagaaagagcctagggagctcttcgtcaaaggcaccatgaaggatattagagagaattttcaagatctcatgcaatactgcgcacaagacgtatgggcaacgcatgaggtctttcaacagcaactccccctctttttggaacgatgtccacatcccgtcactcttgctgggatgttggaaatgggtgtaagttatttgccagtcaatcaaaattgggagagatacttggctgaagcgcagggtacatatgaggaacttcagcgagaaatgaaaaaaagtcttatggatttggccaatgacgcctgccagctgctttccggtgagcggtacaaagaagatccatggctttgggatctcgaatgggatttgcaggaatttaaacagaaaaaggccaagaaggtcaagaaagagccagctacagcctcaaagctccctatagagggagcgggagcaccgggtgatccgatggatcaagaggatttgggaccttgctccgaagaggaggaattccaacaagatgtaatggcaagggcctgcctgcaaaagctcaaagggacaacagaactcttgcccaagaggcctcaacatctgcccggccatccaggttggtatcgcaaactctgtccaaggctggacgatcccgcctggaccccggggccctcccttctgagtctgcagatgagagtgacacctaagctgatggcacttacttgggatgggttccctcttcactattcagagagacacgggtggggatatcttgtcccaggtcgaagggacaatctggcgaagcttcccacaggaactaccttggagagtgcgggcgtagtatgtccttatcgagccatagaaagtctgtatagaaagcattgccttgaacaaggcaaacaacaactcatgcctcaggaagccggcctcgctgaagaatttcttcttactgataactctgctatctggcaaacagtggaggaactggattacctcgaagtcgaggccgaagcgaagatggaaaatttgcgggcggcggtcccaggtcagccccttgctttgacagcacgggggggccctaaggatacccagcccagctatcatcacgggaacggtccatataatgatgttgatatacctggttgttggttttttaaacttcctcataaagatggcaattcatgtaacgttggatccccattcgcaaaggacttcctccccaaaatggaggatgggacgttgcaagcaggtcccggtggagcttctgggccgcgagccctggaaataaataagatgattagcttctggaggaatgcacacaagcgcatttcttcacagatggtagtgtggctgcctcggagtgctttgcccagggctgtgatcagacaccccgattatgatgaagagggactgtacggggcaatattgccccaggtggttacggctggtactattacccgccgggcagttgagccgacctggctgacagcatctaatgccaggcctgatcgcgtgggttctgaacttaaagcaatggtccaagctccgcctggatacacgcttgtcggcgcggacgtggactcccaggaactttggatagcggctgtccttggcgatgcacattttgcagggatgcacgggtgcacggcttttggctggatgacacttcaggggaggaaatcaagggggaccgacctgcactccaagaccgcgacaacggtaggaatctcaagggaacacgctaaaattttcaattatgggagaatatatggtgccggtcaaccgttcgctgagcgccttctgatgcagtttaaccataggctgacacagcaggaggcagcggagaaggcgcagcagatgtacgccgcaactaagggtctcagatggtatcgcctctcagacgagggcgagtggcttgtccgggaattgaacctcccggtcgatcggacggaaggtggttggattagtcttcaggatctcagaaaggtgcagcgggaaacagcacgcaagtctcagtggaagaaatgggaggtggtcgccgagcgggcatggaagggtggaacagaatccgagatgtttaacaagctggagagcatagcaactagtgacatccctaggacaccggtcttgggatgttgcattagccgggcactcgagccatctgccgtacaggaagaattcatgacgtcacgggtcaactgggttgtccagtcttcagccgtcgattatctgcatttgatgctggtcgcgatgaagtggctcttcgaagagtttgctatagatggccgattttgcatctctattcacgatgaggtgcgctacctggtaagggaagaagaccgataccgagccgccctcgcccttcagataacaaatctgcttaccaggtgtatgtttgcatacaagctggggttgaacgaccttccccagtccgtcgctttcttttcagctgttgatatagatcgctgcctgagaaaagaggttacgatggactgcaaaacgccctcaaaccccactggtatggagcgcagatatggcatcccccaaggagaagccctcgacatataccagataattgagctcacgaagggcagcctggagaagagatcccaacctggcccatag

(19) United States
(12) Patent Application Publication
(10) Pub. No.: US 2026/0144894 A1
(43) Pub. Date: May 28, 2026

(54) GENE THERAPY FOR DISEASES CAUSED BY UNBALANCED NUCLEOTIDE POOLS INCLUDING MITOCHONDRIAL DNA DEPLETION SYNDROMES

(71) Applicant: The Trustees of Columbia University in the City of New York, New York, NY (US)

(72) Inventors: Michio Hirano, New York, NY (US); Hasan O. Akman, Haworth, NJ (US); Carlos Lopez-Gomez, Fuengirola (ES)

(21) Appl. No.: 19/332,696

(22) Filed: Sep. 18, 2025

Related U.S. Application Data
(63) Continuation of application No. 17/048,236, filed on Oct. 16, 2020, now Pat. No. 12,419,970.

(51) Int. Cl. A61K 48/00; A61K 38/45; C07K 14/705; C12N 15/86

(52) U.S. Cl. CPC A61K 48/005; A61K 38/45; C07K 14/705; C12N 15/86; C12N 2750/14143; C12Y 207/01021; C12Y 207/01076; C12Y 207/01113

(57) ABSTRACT

The invention relates generally to a method of treatment for a human genetic disease, such as diseases characterized by unbalanced nucleotide pools, e.g., mitochondrial DNA depletion syndromes, and more specifically, thymidine kinase 2 (TK2) deficiency, using gene therapy. The gene therapy may involve administration of one or more constructs, such as a viral vector, containing a nucleic acid encoding a functional protein. The functional protein may correspond to a nuclear gene. For treatment of TK2 deficiency, the gene therapy may involve administration of one or more constructs, such as a viral vector, containing a nucleic acid encoding a functional TK2 enzyme. The treatment may also involve the administration of pharmacological therapy in conjunction with the gene therapy. The treatment protocols of the disclosure, such as those involving gene therapy alone or in combination with pharmacological therapy, can be used to treat, prevent, and/or cure various other disorders of unbalanced nucleoside pools, especially those found in mitochondrial DNA depletion syndrome.

### CROSS-REFERENCE TO RELATED APPLICATIONS

This application is a continuation of U.S. patent application Ser. No. 17/048,236, filed Oct. 16, 2020, which is a U.S. national stage entry under 35 U.S.C. § 371 of PCT International Application No. PCT/US2019/028108, filed Apr. 18, 2019, which claims priority to U.S. Provisional Application No. 62/659,281, filed Apr. 18, 2018, the contents of each of which is incorporated herein by reference in its entirety.

### STATEMENT OF GOVERNMENT SUPPORT

This invention was made with government support under HD080642 awarded by NIH. The government has certain rights in the invention.

### REFERENCE TO SEQUENCE LISTING

This application incorporates-by-reference nucleotide and/or amino acid sequences which are present in the file named “93597-7426_92208-A-PCT-AA_Sequence_Listing_AWG.xml”, which is 56,746 bytes in size, and which was created on Sep. 18, 2025 in the IBM-PC machine format, having an operating system compatibility with MS-Windows, which is contained in the XML file filed Sep. 18, 2025 as part of this application.

### FIELD OF THE INVENTION

The invention relates to the field of gene therapy for the treatment of genetic diseases, including human diseases characterized by unbalanced nucleotide pools. Examples of such diseases are mitochondrial DNA depletion syndromes, such as thymidine kinase 2 (TK2) deficiency.

### BACKGROUND OF THE INVENTION

Mitochondrial diseases are clinically heterogeneous diseases due to defects of the mitochondrial respiratory chain (RC) and oxidative phosphorylation, the biochemical pathways that convert energy in electrons into adenosine triphosphate (ATP). The respiratory chain is comprised of four multi-subunit enzymes (complexes I-IV) that transfer electrons to generate a proton gradient across the inner membrane of mitochondria. The flow of protons down this concentration gradient through complex V subsequently drives ATP synthesis (DiMauro and Schon 2003; DiMauro and Hirano 2005). Coenzyme Q₁₀ (CoQ₁₀) is an essential molecule that shuttles electrons from complexes I and II to complex III. The respiratory chain is unique in eukaryotic, e.g., mammalian, cells by virtue of being controlled by two genomes: one composed of mitochondrial DNA (mtDNA) and another composed of nuclear DNA (nDNA). As a consequence, mutations in either genome can cause mitochondrial diseases.

Mitochondrial DNA depletion syndrome (MDS), which is a subgroup of mitochondrial disease, is a frequent cause of severe childhood encephalomyopathy characterized molecularly by reduction of mitochondrial DNA (mtDNA) copy number in tissues and insufficient synthesis of mitochondrial RC complexes (Hirano, et al. 2001). Mutations in several nuclear genes have been identified as causes of infantile MDS, including TK2, DGUOK, POLO, POLG2, SCLA25A4, MPV17, RRM2B, SUCLA2, SUCLG1, TYMP, OPAL, and C1Oorf2 (PEO1). (Bourdon, et al. 2007; Copeland 2008; Elpeleg, et al. 2005; Mandel, et al. 2001; Naviaux and Nguyen 2004; Ostergaard, et al. 2007; Saada, et al. 2003; Sarzi, et al. 2007; Spinazzola, et al, 2006). In addition, mutations in these nuclear genes can also cause multiple deletions of mtDNA with or without mtDNA depletion (Béhin, et al. 2012; Garone, et al. 2012; Longley, et al. 2006; Nishino, et al. 1999; Paradas, et al. 2012; Ronchi, et al. 2012; Spelbrink, et al. 2001; Tyynismaa, et al. 2012; Tyynismaa, et al. 2009; Van Goethem, et al. 2001).

Most mitochondrial diseases affect multiple body organs and are typically fatal in childhood or early adult life. There are no proven effective treatments or cures for mitochondrial diseases, only supportive therapies. There remains a need for compositions and methods for treating and preventing mitochondrial diseases, such as MDS.

### SUMMARY OF THE INVENTION

The present disclosure relates to compositions and methods that can be used to treat a subject (e.g., a mammalian subject, such as a human subject) that has or is at risk of developing a disease characterized by unbalanced nucleotide pools. Exemplary pathologies that may be treated, prevented, and/or cured using the compositions and methods of the disclosure are mitochondrial diseases, including mitochondrial DNA depletion syndromes, such as deficiencies in a gene selected from TK2, encoding thymidine kinase 2 protein; DGUOK, encoding deoxyguanosine kinase protein; RRM2B, encoding p53R2, p53 inducible subunit of ribonucleotide reductase; TYMP, encoding thymidine phosphorylase; SUCLA2, encoding succinate-CoA ligase ADP-forming beta subunit; SUCLG1, encoding succinate-CoA ligase ADP-forming alpha subunit; MPV17, encoding mitochondrial inner membrane protein MPV17; and POLG, encoding DNA polymerase gamma, catalytic subunit.

Using the compositions and methods of the disclosure, a subject (e.g., a mammalian subject, such as a human subject) that has or is at risk of developing a disease described above may be administered a composition containing a transgene encoding one or more of the foregoing proteins. The composition may be a vector, for example, a viral vector, such as an adeno-associated virus (AAV) vector. In some embodiments, the subject is administered a second composition containing a transgene encoding one or more of the foregoing proteins. The second composition may be a vector, for example, a viral vector, such as an AAV vector. In some embodiments, the subject is additionally administered one or more subsequent compositions, such as a third, fourth, fifth, sixth, seventh, eighth, ninth, and/or tenth composition, containing a transgene encoding one or more of the foregoing proteins. The one or more subsequent compositions may each be, independently, a vector, for example, a viral vector, such as an AAV vector.

In a first aspect, the disclosure features a method of treating, preventing, and/or curing a disease or disorder characterized by unbalanced nucleotide pools in a subject in need thereof. The method includes administering to the subject a therapeutically effective amount of a composition containing a transgene encoding thymidine kinase 2 (TK2), deoxyguanosine kinase (dGK), thymidine phosphorylase (TP), p53 inducible small subunit of ribonucleotide reductase (p53R2), succinyl-CoA ligase ADP-forming subunit beta (SUCLA2), succinyl-CoA ligase GDP-forming subunit alpha (SUCLG1), mitochondrial inner membrane protein MPV17 (MPV17), and/or DNA polymerase subunit gamma (POLG).

In a further aspect, the disclosure features a method of restoring enzyme activity in a subject having a disease or disorder characterized by unbalanced nucleotide pools. The method includes administering to the subject a therapeutically effective amount of a composition containing a transgene encoding TK2, dGK, TP, p53R2, SUCLA2, SUCLG1, MPV17, and/or POLG.

In an additional aspect, the disclosure features a method of alleviating one or more symptoms associated with a disease or disorder characterized by unbalanced nucleotide pools in a subject in need thereof. The method includes administering to the subject a therapeutically effective amount of a composition containing a transgene encoding TK2, dGK, TP, p53R2, SUCLA2, SUCLG1, MPV17, and/or POLG.

In any of the aspects of this invention, the disclosure also provides a composition as described herein for use in a method as described herein. The disclosure also provides the use of a composition as described herein for the manufacture of a medicament for a method as described herein. The transgene may encode TK2, dGK, TP, p53R2, SUCLA2, SUCLG1, MPV17, and/or POLG. For example, the transgene may encode TK2, dGK, TP, p53R2, SUCLA2, SUCLG1 and/or MPV17.

As part of the foregoing aspects, the disclosure therefore also provides a composition containing a transgene encoding TK2, dGK, TP, p53R2, SUCLA2, SUCLG1, MPV17, and/or POLG for use in treating, preventing, and/or curing a disease or disorder characterized by unbalanced nucleotide pools. Also provided is a composition containing a transgene encoding TK2, dGK, TP, p53R2, SUCLA2, SUCLG1, MPV17, and/or POLG for use in restoring enzyme activity in a subject having a disease or disorder characterized by unbalanced nucleotide pools. Furthermore provided is a composition containing a transgene encoding TK2, dGK, TP, p53R2, SUCLA2, SUCLG1, MPV17, and/or POLG for use in alleviating one or more symptoms associated with a disease or disorder characterized by unbalanced nucleotide pools.

As part of the foregoing aspects, the disclosure also provides the use of a composition containing a transgene encoding TK2, dGK, TP, p53R2, SUCLA2, SUCLG1, MPV17, and/or POLG for the manufacture of a medicament for treating, preventing, and/or curing a disease or disorder characterized by unbalanced nucleotide pools. Also provided is the use of a composition containing a transgene encoding TK2, dGK, TP, p53R2, SUCLA2, SUCLG1, MPV17, and/or POLG for the manufacture of a medicament for restoring enzyme activity in a subject having a disease or disorder characterized by unbalanced nucleotide pools. Furthermore provided is the use of a composition containing a transgene encoding TK2, dGK, TP, p53R2, SUCLA2, SUCLG1, MPV17, and/or POLG for the manufacture of a medicament for alleviating one or more symptoms associated with a disease or disorder characterized by unbalanced nucleotide pools.

In some embodiments of any of the foregoing aspects, the disease or disorder is a mitochondrial disease, such as a mitochondrial DNA depletion syndrome (MDS). In some embodiments, the MDS is a myopathic MDS characterized by one or more mutations in an endogenous gene encoding TK2. In some embodiments, the MDS is an encephalomyopathic form characterized by one or more mutations in an endogenous gene encoding SUCLA2. In some embodiments, the MDS is a neurogastrointestinal encephalopathic form characterized by one or more mutations in an endogenous gene encoding TP. In some embodiments, the MDS is a hepatopathic form characterized by one or more mutations in an endogenous gene encoding dGK, MPV17, and/or POLG.

In some embodiments, the transgene encodes TK2. The TK2 may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1). In some embodiments, the TK2 has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TK2 has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding TK2 has a nucleic acid sequence that is at least 70% identical to the nucleic acid sequence of SEQ ID NO: 2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 85% identical to the nucleic acid sequence of SEQ ID NO: 2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 90% identical to the nucleic acid sequence of SEQ ID NO: 2 (e.g., a nucleic acid sequence that is 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 95% identical to the nucleic acid sequence of SEQ ID NO: 2 (e.g., a nucleic acid sequence that is 95%, 96%, 97%, 98%, 99%, or 100% identical to the nucleic acid sequence of SEQ ID NO: 2). In some embodiments, the transgene encoding TK2 is codon optimized to increase efficiency. In some embodiments, the nucleic acid of the transgene encoding TK2 is at least at least 70% identical to coding sequence of the transgene that encodes TK2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TK2 has the nucleic acid sequence of SEQ ID NO: 17.

In some embodiments, the transgene encodes dGK. The dGK may have an amino acid 10 sequence that is at least 85% identical to the amino acid sequence of SEQ ID NO: 3 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 90% identical to the amino acid sequence of SEQ ID NO: 3 (e.g., an amino acid sequence that is 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 95% identical to the amino acid sequence of SEQ ID NO: 3 (e.g., an amino acid sequence that is 95%, 96%, 97%, 98%, 99%, or 100% identical to the amino acid sequence of SEQ ID NO: 3).

In some embodiments, the dGK has an amino acid sequence that differs from SEQ ID NO: 3 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the dGK has an amino acid sequence that differs from SEQ ID NO: 3 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding dGK has a nucleic acid sequence that is at least 70% identical to the nucleic acid sequence of SEQ ID NO: 4 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 85% identical to the nucleic acid sequence of SEQ ID NO: 4 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 90% identical to the nucleic acid sequence of SEQ ID NO: 4 (e.g., a nucleic acid sequence that is 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 95% identical to the nucleic acid sequence of SEQ ID NO: 4 (e.g., a nucleic acid sequence that is 95%, 96%, 97%, 98%, 99%, or 100% identical to the nucleic acid sequence of SEQ ID NO: 4). In some embodiments, the transgene encoding dGK is codon optimized to increase efficiency. In some embodiments, the nucleic acid of the transgene encoding dGK is at least at least 70% identical to coding sequence of the transgene that encodes dGK (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding dGK has the nucleic acid sequence of SEQ ID NO: 18.

In some embodiments, the transgene encodes TP. The TP may have an amino acid sequence that is at least 85% identical to the amino acid sequence of SEQ ID NO: 5 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 90% identical to the amino acid sequence of SEQ ID NO: 5 (e.g., an amino acid sequence that is 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 95% identical to the amino acid sequence of SEQ ID NO: 5 (e.g., an amino acid sequence that is 95%, 96%, 97%, 98%, 99%, or 100% identical to the amino acid sequence of SEQ ID NO: 5).

In some embodiments, the TP has an amino acid sequence that differs from SEQ ID NO: 5 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TP has an amino acid sequence that differs from SEQ ID NO: 5 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding TP has a nucleic acid sequence that is at least 70% identical to the nucleic acid sequence of SEQ ID NO: 6 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 85% identical to the nucleic acid sequence of SEQ ID NO: 6 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 90% identical to the nucleic acid sequence of SEQ ID NO: 6 (e.g., a nucleic acid sequence that is 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 95% identical to the nucleic acid sequence of SEQ ID NO: 6 (e.g., a nucleic acid sequence that is 95%, 96%, 97%, 98%, 99%, or 100% identical to the nucleic acid sequence of SEQ ID NO: 6). In some embodiments, the transgene encoding TP is codon optimized to increase efficiency. In some embodiments, the nucleic acid of the transgene encoding TP is at least at least 70% identical to coding sequence of the transgene that encodes TP (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TP has the nucleic acid sequence of SEQ ID NO: 19.

In some embodiments, the transgene encodes p53R2. The p53R2 may have an amino acid sequence that is at least 85% identical to the amino acid sequence of SEQ ID NO: 7 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 90% identical to the amino acid sequence of SEQ ID NO: 7 (e.g., an amino acid sequence that is 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 95% identical to the amino acid sequence of SEQ ID NO: 7 (e.g., an amino acid sequence that is 95%, 96%, 97%, 98%, 99%, or 100% identical to the amino acid sequence of SEQ ID NO: 7). In some embodiments, the p53R2 has an amino acid sequence that differs from SEQ ID NO: 7 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the p53R2 has an amino acid sequence that differs from SEQ ID NO: 7 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding p53R2 has a nucleic acid sequence that is at least 70% identical to the nucleic acid sequence of SEQ ID NO: 8 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 85% identical to the nucleic acid sequence of SEQ ID NO: 8 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 90% identical to the nucleic acid sequence of SEQ ID NO: 8 (e.g., a nucleic acid sequence that is 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 95% identical to the nucleic acid sequence of SEQ ID NO: 8 (e.g., a nucleic acid sequence that is 95%, 96%, 97%, 98%, 99%, or 100% identical to the nucleic acid sequence of SEQ ID NO: 8). In some embodiments, the transgene encoding p53R2 is codon optimized to increase efficiency. In some embodiments, the nucleic acid of the transgene encoding p53R2 is at least at least 70% identical to coding sequence of the transgene that encodes p53R2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding p53R2 has the nucleic acid sequence of SEQ ID NO: 20.

In some embodiments, the transgene encodes SUCLA2. The SUCLA2 may have an amino acid sequence that is at least 85% identical to the amino acid sequence of SEQ ID NO: 9 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 90% identical to the amino acid sequence of SEQ ID NO: 9 (e.g., an amino acid sequence that is 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 95% identical to the amino acid sequence of SEQ ID NO: 9 (e.g., an amino acid sequence that is 95%, 96%, 97%, 98%, 99%, or 100% identical to the amino acid sequence of SEQ ID NO: 9). In some embodiments, the SUCLA2 has an amino acid sequence that differs from SEQ ID NO: 9 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLA2 has an amino acid sequence that differs from SEQ ID NO: 9 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding SUCLA2 has a nucleic acid sequence that is at least 70% identical to the nucleic acid sequence of SEQ ID NO: 10 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 85% identical to the nucleic acid sequence of SEQ ID NO: 10 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 90% identical to the nucleic acid sequence of SEQ ID NO: 10 (e.g., a nucleic acid sequence that is 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 95% identical to the nucleic acid sequence of SEQ ID NO: 10 (e.g., a nucleic acid sequence that is 95%, 96%, 97%, 98%, 99%, or 100% identical to the nucleic acid sequence of SEQ ID NO: 10). In some embodiments, the transgene encoding SUCLA2 is codon optimized to increase efficiency. In some embodiments, the nucleic acid of the transgene encoding SUCLA2 is at least at least 70% identical to coding sequence of the transgene that encodes SUCLA2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding SUCLA2 has the nucleic acid sequence of SEQ ID NO: 21.

In some embodiments, the transgene encodes SUCLG1. The SUCLG1 may have an amino acid sequence that is at least 85% identical to the amino acid sequence of SEQ ID NO: 11 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 90% identical to the amino acid sequence of SEQ ID NO: 11 (e.g., an amino acid sequence that is 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 95% identical to the amino acid sequence of SEQ ID NO: 11 (e.g., an amino acid sequence that is 95%, 96%, 97%, 98%, 99%, or 100% identical to the amino acid sequence of SEQ ID NO: 11). In some embodiments, the SUCLG1 has an amino acid sequence that differs from SEQ ID NO: 11 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLG1 has an amino acid sequence that differs from SEQ ID NO: 11 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding SUCLG1 has a nucleic acid sequence that is at least 70% identical to the nucleic acid sequence of SEQ ID NO: 12 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 85% identical to the nucleic acid sequence of SEQ ID NO: 12 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 90% identical to the nucleic acid sequence of SEQ ID NO: 12 (e.g., a nucleic acid sequence that is 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 95% identical to the nucleic acid sequence of SEQ ID NO: 12 (e.g., a nucleic acid sequence that is 95%, 96%, 97%, 98%, 99%, or 100% identical to the nucleic acid sequence of SEQ ID NO: 12). In some embodiments, the transgene encoding SUCLG1 is codon optimized to increase efficiency. In some embodiments, the nucleic acid of the transgene encoding SUCLG1 is at least at least 70% identical to coding sequence of the transgene that encodes SUCLG1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding SUCLG1 has the nucleic acid sequence of SEQ ID NO: 22.

In some embodiments, the transgene encodes MPV17. The MPV17 may have an amino acid sequence that is at least 85% identical to the amino acid sequence of SEQ ID NO: 13 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 90% identical to the amino acid sequence of SEQ ID NO: 13 (e.g., an amino acid sequence that is 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 95% identical to the amino acid sequence of SEQ ID NO: 13 (e.g., an amino acid sequence that is 95%, 96%, 97%, 98%, 99%, or 100% identical to the amino acid sequence of SEQ ID NO: 13). In some embodiments, the MPV17 has an amino acid sequence that differs from SEQ ID NO: 13 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the MPV17 has an amino acid sequence that differs from SEQ ID NO: 13 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding MPV17 has a nucleic acid sequence that is at least 70% identical to the nucleic acid sequence of SEQ ID NO: 14 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 85% identical to the nucleic acid sequence of SEQ ID NO: 14 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 90% identical to the nucleic acid sequence of SEQ ID NO: 14 (e.g., a nucleic acid sequence that is 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 95% identical to the nucleic acid sequence of SEQ ID NO: 14 (e.g., a nucleic acid sequence that is 95%, 96%, 97%, 98%, 99%, or 100% identical to the nucleic acid sequence of SEQ ID NO: 14). In some embodiments, the transgene encoding MPV17 is codon optimized to increase efficiency. In some embodiments, the nucleic acid of the transgene encoding MPV17 is at least at least 70% identical to coding sequence of the transgene that encodes MPV17 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding MPV17 has the nucleic acid sequence of SEQ ID NO: 23.

In some embodiments, the transgene encodes POLG. The POLG may have an amino acid sequence that is at least 85% identical to the amino acid sequence of SEQ ID NO: 15 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 90% identical to the amino acid sequence of SEQ ID NO: 15 (e.g., an amino acid sequence that is 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 95% identical to the amino acid sequence of SEQ ID NO: 15 (e.g., an amino acid sequence that is 95%, 96%, 97%, 98%, 99%, or 100% identical to the amino acid sequence of SEQ ID NO: 15).

In some embodiments, the POLG has an amino acid sequence that differs from SEQ ID NO: 15 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the POLG has an amino acid sequence that differs from SEQ ID NO: 15 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding POLG has a nucleic acid sequence that is at least 70% identical to the nucleic acid sequence of SEQ ID NO: 16 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 85% identical to the nucleic acid sequence of SEQ ID NO: 16 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 90% identical to the nucleic acid sequence of SEQ ID NO: 16 (e.g., a nucleic acid sequence that is 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 95% identical to the nucleic acid sequence of SEQ ID NO: 16 (e.g., a nucleic acid sequence that is 95%, 96%, 97%, 98%, 99%, or 100% identical to the nucleic acid sequence of SEQ ID NO: 16). In some embodiments, the transgene encoding POLG is codon optimized to increase efficiency. In some embodiments, the nucleic acid of the transgene encoding POLG is at least at least 70% identical to coding sequence of the transgene that encodes POLG (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In 30 some embodiments, the nucleic acid sequence of the transgene encoding POLG has the nucleic acid sequence of SEQ ID NO: 24.

In another aspect, the disclosure features a method of treating, preventing, and/or curing TK2 deficiency in a subject in need thereof. The method includes administering to the subject a therapeutically effective amount of a composition containing a transgene encoding TK2. In a further aspect, the disclosure features a method of restoring TK2 enzyme activity in a subject in need thereof. The method includes administering to the subject a therapeutically effective amount of a composition containing a transgene encoding TK2.

In another aspect, the disclosure features a method of alleviating one or more symptoms associated with a TK2 deficiency in a subject in need thereof. The method includes administering to the subject a therapeutically effective amount of a composition containing a transgene encoding TK2.

As part of these aspects, the disclosure also provides a composition containing a transgene encoding TK2 for use in treating, preventing, and/or curing TK2 deficiency. Also provided is a composition containing a transgene encoding TK2 for use in restoring TK2 enzyme activity in a subject having a TK2 deficiency. Furthermore provided is a composition containing a transgene encoding TK2 for use in alleviating one or more symptoms associated with a TK2 deficiency. Typically the subject has a TK2 deficiency.

As part of these aspects, the disclosure also provides the use of a composition containing a transgene encoding TK2 for the manufacture of a medicament for treating, preventing, and/or curing TK2 deficiency. Also provided is the use of a composition containing a transgene encoding TK2 for the manufacture of a medicament for restoring TK2 enzyme activity in a subject having a TK2 deficiency. Furthermore provided is the use of a composition containing a transgene encoding TK2 for the manufacture of a medicament for alleviating one or more symptoms associated with a TK2 deficiency. Typically the subject has a TK2 deficiency.

In some embodiments of any of the preceding three aspects, the subject has or is at risk of developing a MDS. The TK2 may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1). In some embodiments, the TK2 has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TK2 has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene encoding TK2 has a nucleic acid sequence that is at least 70% identical to the nucleic acid sequence of SEQ ID NO: 2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 85% identical to the nucleic acid sequence of SEQ ID NO: 2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 90% identical to the nucleic acid sequence of SEQ ID NO: 2 (e.g., a nucleic acid sequence that is 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 95% identical to the nucleic acid sequence of SEQ ID NO: 2 (e.g., a nucleic acid sequence that is 95%, 96%, 97%, 98%, 99%, or 100% identical to the nucleic acid sequence of SEQ ID NO: 2). In some embodiments, the transgene encoding TK2 is codon optimized to increase efficiency. In some embodiments, the nucleic acid of the transgene encoding TK2 is at least at least 70% identical to coding sequence of the transgene that encodes TK2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TK2 has the nucleic acid 30 sequence of SEQ ID NO: 17.

In some embodiments of any of the above aspects of the disclosure, the composition comprises a vector, such as a viral vector. The viral vector may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

In some embodiments, the viral vector is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is 20 AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAVrh74, AAVrh.8, or AAVrh.10.

In certain embodiments, the viral vector is AAV9. For example, the composition may comprise AAV9 comprising a nucleic acid sequence comprising a transgene encoding TK2, dGK, TP, p53R2, SUCLA2, SUCLG1, MPV17, and/or POLG. In certain embodiments, the composition comprises AAV9 comprising a nucleic acid sequence comprising a transgene encoding TK2, dGK, TP, p53R2, SUCLA2, SUCLG1 or MPV17. In a particular embodiment, the composition comprises AAV9 comprising a nucleic acid sequence comprising a transgene encoding TK2.

In certain embodiments, the viral vector is an AAV and the transgene is TK2. For example, the composition may comprise a recombinant AAV (rAAV), such as AAV9, comprising a nucleic acid sequence comprising a transgene encoding a functional TK2 protein. In some embodiments of any of the above aspects of the disclosure, the composition is a liposome, vesicle, synthetic vesicle, exosome, synthetic exosome, dendrimer, or nanoparticle.

In some embodiments of any of the above aspects of the disclosure, the transgene is operably linked to a promoter that induces expression of the transgene in a muscle cell. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments of any of the above aspects of the disclosure, the transgene is operably linked to an enhancer that induces expression of the transgene in a muscle cell.

Exemplary enhancers that may be used in conjunction with the compositions and methods of the disclosure are a CMV enhancer, a myocyte enhancer factor 2 (MEF2) enhancer, and a MyoD enhancer.

In some embodiments, the composition is administered to the subject as soon as, or immediately after, the subject is diagnosed as having a deficiency in an endogenous gene encoding TK2, dGK, TP, p53R2, SUCLA2, SUCLG1, MPV17, and/or POLG. In some embodiments, the composition is administered to the subject as soon as, or immediately after, the subject is diagnosed as having a deficiency in an endogenous gene encoding TK2.

In some embodiments, the method further includes administering to the subject a therapeutically effective amount of a second composition containing a transgene encoding TK2, dGK, TP, p53R2, SUCLA2, SUCLG1, MPV17, and/or POLG.

In some embodiments, the transgene of the second composition encodes TK2. The TK2 may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1). In some embodiments, the TK2 has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TK2 has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding TK2 has a nucleic acid sequence that is at least 70% identical to the nucleic acid sequence of SEQ ID NO: 2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene of the second composition encoding TK2 has a nucleic acid sequence that is at least 85% identical to the nucleic acid sequence of SEQ ID NO: 2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 90% identical to the nucleic acid sequence of SEQ ID NO: 2 (e.g., a nucleic acid sequence that is 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 95% identical to the nucleic acid sequence of SEQ ID NO: 2 (e.g., a nucleic acid sequence that is 95%, 96%, 97%, 98%, 99%, or 100% identical to the nucleic acid sequence of SEQ ID NO: 2). In some embodiments, the transgene encoding TK2 is codon optimized to increase efficiency. In some embodiments, the nucleic acid of the transgene encoding TK2 is at least at least 70% identical to coding sequence of the transgene that encodes TK2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding 30 TK2 has the nucleic acid sequence of SEQ ID NO: 17.

In some embodiments, the transgene of the second composition encodes dGK. The dGK may have an amino acid sequence that is at least 85% identical to the amino acid sequence of SEQ ID NO: 3 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 90% identical to the amino acid sequence of SEQ ID NO: 3 (e.g., an amino acid sequence that is 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 95% identical to the amino acid sequence of SEQ ID NO: 3 (e.g., an amino acid sequence that is 95%, 96%, 97%, 98%, 99%, or 100% identical to the amino acid sequence of SEQ ID NO: 3). In some embodiments, the dGK has an amino acid sequence that differs from SEQ ID NO: 3 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the dGK has an amino acid sequence that differs from SEQ ID NO: 3 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding dGK has a nucleic acid sequence that is at least 70% identical to the nucleic acid sequence of SEQ ID NO: 4 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments, the transgene of the second composition encoding dGK has a nucleic acid sequence that is at least 85% identical to the nucleic acid sequence of SEQ ID NO: 4 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 90% identical to the nucleic acid sequence of SEQ ID NO: 4 (e.g., a nucleic acid sequence that is 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 95% identical to the nucleic acid sequence of SEQ ID NO: 4 (e.g., a nucleic acid sequence that is 95%, 96%, 97%, 98%, 99%, or 100% identical to the nucleic acid sequence of SEQ ID NO: 4). In some embodiments, the transgene encoding dGK is codon optimized to increase efficiency. In some embodiments, the nucleic acid of the transgene encoding dGK is at least at least 70% identical to coding sequence of the transgene that encodes dGK (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding dGK has the nucleic acid sequence of SEQ ID NO: 18.

In some embodiments, the transgene of the second composition encodes TP. The TP may have an amino acid sequence that is at least 85% identical to the amino acid sequence of SEQ ID NO: 5 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 90% identical to the amino acid sequence of SEQ ID NO: 5 (e.g., an amino acid sequence that is 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 95% identical to the amino acid sequence of SEQ ID NO: 5 (e.g., an amino acid sequence that is 95%, 96%, 97%, 98%, 99%, or 100% identical to the amino acid sequence of SEQ ID NO: 5). In some embodiments, the TP has an amino acid sequence that differs from SEQ ID NO: 5 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TP has an amino acid sequence that differs from SEQ ID NO: 5 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding TP has a nucleic acid sequence that is at least 70% identical to the nucleic acid sequence of SEQ ID NO: 6 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, the transgene of the second composition encoding TP has a nucleic acid sequence that is at least 85% identical to the nucleic acid sequence of SEQ ID NO: 6 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 90% identical to the nucleic acid sequence of SEQ ID NO: 6 (e.g., a nucleic acid sequence that is 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 95% identical to the nucleic acid sequence of SEQ ID NO: 6 (e.g., a nucleic acid sequence that is 95%, 96%, 97%, 98%, 99%, or 100% identical to the nucleic acid sequence of SEQ ID NO: 6). In some embodiments, the transgene encoding TP is codon optimized to increase efficiency. In some embodiments, the nucleic acid of the transgene encoding TP is at least at least 70% identical to coding sequence of the transgene that encodes TP (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TP has the nucleic acid sequence of SEQ ID NO: 19.

In some embodiments, the transgene of the second composition encodes p53R2. The p53R2 may have an amino acid sequence that is at least 85% identical to the amino acid sequence of SEQ ID NO: 7 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 90% identical to the amino acid sequence of SEQ ID NO: 7 (e.g., an amino acid sequence that is 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 95% identical to the amino acid sequence of SEQ ID NO: 7 (e.g., an amino acid sequence that is 95%, 96%, 97%, 98%, 99%, or 100% identical to the amino acid sequence of SEQ ID NO: 7). In some embodiments, the p53R2 has an amino acid sequence that differs from SEQ ID NO: 7 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the p53R2 has an amino acid sequence that differs from SEQ ID NO: 7 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding p53R2 has a nucleic acid sequence that is at least 70% identical to the nucleic acid sequence of SEQ ID NO: 8 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the transgene of the second composition encoding p53R2 has a nucleic acid sequence that is at least 85% identical to the nucleic acid sequence of SEQ ID NO: 8 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 90% identical to the nucleic acid sequence of SEQ ID NO: 8 (e.g., a nucleic acid sequence that is 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 95% identical to the nucleic acid sequence of SEQ ID NO: 8 (e.g., a nucleic acid sequence that is 95%, 96%, 97%, 98%, 99%, or 100% identical to the nucleic acid sequence of SEQ ID NO: 8). In some embodiments, the transgene encoding p53R2 is codon optimized to increase efficiency. In some embodiments, the nucleic acid of the transgene encoding p53R2 is at least at least 70% identical to coding sequence of the transgene that encodes p53R2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding p53R2 has the nucleic acid sequence of SEQ ID NO: 20.

In some embodiments, the transgene of the second composition encodes SUCLA2. The SUCLA2 may have an amino acid sequence that is at least 85% identical to the amino acid sequence of SEQ ID NO: 9 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 90% identical to the amino acid sequence of SEQ ID NO: 9 (e.g., an amino acid sequence that is 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 95% identical to the amino acid sequence of SEQ ID NO: 9 (e.g., an amino acid sequence that is 95%, 96%, 97%, 98%, 99%, or 100% identical to the amino acid sequence of SEQ ID NO: 9). In some embodiments, the SUCLA2 has an amino acid sequence that differs from SEQ ID NO: 9 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLA2 has an amino acid sequence that differs from SEQ ID NO: 9 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding SUCLA2 has a nucleic acid sequence that is at least 70% identical to the nucleic acid sequence of SEQ ID NO: 10 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments, the transgene of the second composition encoding SUCLA2 has a nucleic acid sequence that is at least 85% identical to the nucleic acid sequence of SEQ ID NO: 10 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 90% identical to the nucleic acid sequence of SEQ ID NO: 10 (e.g., a nucleic acid sequence that is 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 95% identical to the nucleic acid sequence of SEQ ID NO: 10 (e.g., a nucleic acid sequence that is 95%, 96%, 97%, 98%, 99%, or 100% identical to the nucleic acid sequence of SEQ ID NO: 10). In some embodiments, the transgene encoding SUCLA2 is codon optimized to increase efficiency. In some embodiments, the nucleic acid of the transgene encoding SUCLA2 is at least at least 70% identical to coding sequence of the transgene that encodes SUCLA2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid 30 sequence of the transgene encoding SUCLA2 has the nucleic acid sequence of SEQ ID NO: 21.

In some embodiments, the transgene of the second composition encodes SUCLG1. The SUCLG1 may have an amino acid sequence that is at least 85% identical to the amino acid sequence of SEQ ID NO: 11 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 90% identical to the amino acid sequence of SEQ ID NO: 11 (e.g., an amino acid sequence that is 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 95% identical to the amino acid sequence of SEQ ID NO: 11 (e.g., an amino acid sequence that is 95%, 96%, 97%, 98%, 99%, or 100% identical to the amino acid sequence of SEQ ID NO: 11). In some embodiments, the SUCLG1 has an amino acid sequence that differs from SEQ ID NO: 11 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLG1 has an amino acid sequence that differs from SEQ ID NO: 11 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding SUCLG1 has a nucleic acid sequence that is at least 70% identical to the nucleic acid sequence of SEQ ID NO: 12 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments, the transgene of the second composition encoding SUCLG1 has a nucleic acid sequence that is at least 85% identical to the nucleic acid sequence of SEQ ID NO: 12 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 90% identical to the nucleic acid sequence of SEQ ID NO: 12 (e.g., a nucleic acid sequence that is 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 95% identical to the nucleic acid sequence of SEQ ID NO: 12 (e.g., a nucleic acid sequence that is 95%, 96%, 97%, 98%, 99%, or 100% identical to the nucleic acid sequence of SEQ ID NO: 12). In some embodiments, the transgene encoding SUCLG1 is codon optimized to increase efficiency. In some embodiments, the nucleic acid of the transgene encoding SUCLG1 is at least at least 70% identical to coding sequence of the transgene that encodes SUCLG1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding SUCLG1 has the nucleic acid sequence of SEQ ID NO: 22.

In some embodiments, the transgene of the second composition encodes MPV17. The MPV17 may have an amino acid sequence that is at least 85% identical to the amino acid sequence of SEQ ID NO: 13 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 90% identical to the amino acid sequence of SEQ ID NO: 13 (e.g., an amino acid sequence that is 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 95% identical to the amino acid sequence of SEQ ID NO: 13 (e.g., an amino acid sequence that is 95%, 96%, 97%, 98%, 99%, or 100% identical to the amino acid sequence of SEQ ID NO: 13). In some embodiments, the MPV17 has an amino acid sequence that differs from SEQ ID NO: 13 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the MPV17 has an amino acid sequence that differs from SEQ ID NO: 13 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding MPV17 has a nucleic acid sequence that is at least 70% identical to the nucleic acid sequence of SEQ ID NO: 14 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments, the transgene of the second composition encoding MPV17 has a nucleic acid sequence that is at least 85% identical to the nucleic acid sequence of SEQ ID NO: 14 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 90% identical to the nucleic acid sequence of SEQ ID NO: 14 (e.g., a nucleic acid sequence that is 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 95% identical to the nucleic acid sequence of SEQ ID NO: 14 (e.g., a nucleic acid sequence that is 95%, 96%, 97%, 98%, 99%, or 100% identical to the nucleic acid sequence of SEQ ID NO: 14). In some embodiments, the transgene encoding MPV17 is codon optimized to increase efficiency. In some embodiments, the nucleic acid of the transgene encoding MPV17 is at least at least 70% identical to coding sequence of the transgene that encodes MPV17 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding MPV17 has the nucleic acid sequence of SEQ ID NO: 23.

In some embodiments, the transgene of the second composition encodes POLG. The POLG may have an amino acid sequence that is at least 85% identical to the amino acid sequence of SEQ ID NO: 15 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 90% identical to the amino acid sequence of SEQ ID NO: 15 (e.g., an amino acid sequence that is 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 95% identical to the amino acid sequence of SEQ ID NO: 15 (e.g., an amino acid sequence that is 95%, 96%, 97%, 98%, 99%, or 100% identical to the amino acid sequence of SEQ ID NO: 15). In some embodiments, the POLG has an amino acid sequence that differs from SEQ ID NO: 15 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the POLG has an amino acid sequence that differs from SEQ ID NO: 15 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments, the transgene of the second composition encoding POLG has a nucleic acid sequence that is at least 70% identical to the nucleic acid sequence of SEQ ID NO: 16 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments, the transgene of the second composition encoding POLG has a nucleic acid sequence that is at least 85% identical to the nucleic acid sequence of SEQ ID NO: 16 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 90% identical to the nucleic acid sequence of SEQ ID NO: 16 (e.g., a nucleic acid sequence that is 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 95% identical to the nucleic acid sequence of SEQ ID NO: 16 (e.g., a nucleic acid sequence that is 95%, 96%, 97%, 98%, 99%, or 100% identical to the nucleic acid sequence of SEQ ID NO: 16). In some embodiments, the transgene encoding POLG is codon optimized to increase efficiency. In some embodiments, the nucleic acid of the transgene encoding POLG is at least at least 70% identical to coding sequence of the transgene that encodes POLG (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding POLG has the nucleic acid sequence of SEQ ID NO: 24.

In some embodiments, the second composition is a vector, such as a viral vector. The viral vector may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

In some embodiments, the second composition is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the second composition is a pseudotyped AAV, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the second composition is an AAV that contains a recombinant capsid protein, such as a capsid protein containing a chimera of one or more of capsid proteins from AAV1, AAV2, AAV3, AAV4, AAV5, AAV6, AAV7, AAV8, AAV9, AAVrh74, AAVrh.8, or AAVrh.10.

In some embodiments, the second composition is a liposome, vesicle, synthetic vesicle, exosome, synthetic exosome, dendrimer, or nanoparticle.

In some embodiments, the transgene of the second composition is operably linked to a promoter that induces expression of the transgene in a muscle cell. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments, the transgene of the second composition is operably linked to an enhancer that induces expression of the transgene in a muscle cell. Exemplary enhancers that may be used in conjunction with the compositions and methods of the disclosure are a CMV enhancer, a myocyte enhancer factor 2 (MEF2) enhancer, and a MyoD enhancer.

In some embodiments, the second composition is administered to the subject after administration of the first composition to the subject. The second composition may be administered to the subject, for example, within one or more days or weeks of administration of the first composition to the subject. In some embodiments, the second composition is administered to the subject at least one month after administration of the first composition to the subject. In some embodiments, administration of the first composition continues while the second composition is administered to the subject.

In some embodiments, the method further includes administering to the subject a therapeutically effective amount of a third composition containing a pharmacological agent.

The pharmacological agent may be, for example, deoxycytidine (dC), deoxythymidine (dT), deoxyadenosine (dA), deoxyguanosine (dG), deoxycytidine monophosphate (dCMP), deoxythymidine monophosphate (TMP), deoxyadenosine monophosphate (dAMP), deoxyguanosine monophosphate (dGMP), or a mixture thereof. In some embodiments, the pharmacological agent is dC, dT, or a mixture thereof.

In some embodiments, the third composition is administered to the subject after administration of the first composition to the subject. The third composition may be administered to the subject, for example, within one or more days or weeks of administration of the first composition to the subject. In some embodiments, the third composition is administered to the subject at least one month after administration of the first composition to the subject.

In some embodiments, the third composition is administered to the subject after administration of the second composition to the subject. The third composition may be administered to the subject, for example, within one or more days or weeks of administration of the second composition to the subject. In some embodiments, the third composition is administered to the subject at least one month after administration of the second composition to the subject.

In some embodiments, administration of the first composition and administration of the second composition continue while the third composition is administered to the subject.

In some embodiments, the third composition is administered to the subject in an amount of from about 100 mg/kg/day to about 1,000 mg/kg/day (e.g., about 100 mg/kg/day, 105 mg/kg/day, 110 mg/kg/day, 115 mg/kg/day, 120 mg/kg/day, 125 mg/kg/day, 130 mg/kg/day, 135 mg/kg/day, 140 mg/kg/day, 145 mg/kg/day, 150 mg/kg/day, 155 mg/kg/day, 160 mg/kg/day, 165 mg/kg/day, 170 mg/kg/day, 175 mg/kg/day, 180 mg/kg/day, 185 mg/kg/day, 190 mg/kg/day, 195 mg/kg/day, 200 mg/kg/day, 205 mg/kg/day, 210 mg/kg/day, 215 mg/kg/day, 220 mg/kg/day, 225 mg/kg/day, 230 mg/kg/day, 235 mg/kg/day, 240 mg/kg/day, 245 mg/kg/day, 250 mg/kg/day, 255 mg/kg/day, 260 mg/kg/day, 265 mg/kg/day, 270 mg/kg/day, 275 mg/kg/day, 280 mg/kg/day, 285 mg/kg/day, 290 mg/kg/day, 295 mg/kg/day, 300 mg/kg/day, 305 mg/kg/day, 310 mg/kg/day, 315 mg/kg/day, 320 mg/kg/day, 325 mg/kg/day, 330 mg/kg/day, 335 mg/kg/day, 340 mg/kg/day, 345 mg/kg/day, 350 mg/kg/day, 355 mg/kg/day, 360 mg/kg/day, 365 mg/kg/day, 370 mg/kg/day, 375 mg/kg/day, 380 mg/kg/day, 385 mg/kg/day, 390 mg/kg/day, 395 mg/kg/day, 400 mg/kg/day, 405 mg/kg/day, 410 mg/kg/day, 415 mg/kg/day, 420 mg/kg/day, 425 mg/kg/day, 430 mg/kg/day, 435 mg/kg/day, 440 mg/kg/day, 445 mg/kg/day, 450 mg/kg/day, 455 mg/kg/day, 460 mg/kg/day, 465 mg/kg/day, 470 mg/kg/day, 475 mg/kg/day, 480 mg/kg/day, 485 mg/kg/day, 490 mg/kg/day, 495 mg/kg/day, 500 mg/kg/day, 505 mg/kg/day, 510 mg/kg/day, 515 mg/kg/day, 520 mg/kg/day, 525 mg/kg/day, 530 mg/kg/day, 535 mg/kg/day, 540 mg/kg/day, 545 mg/kg/day, 550 mg/kg/day, 555 mg/kg/day, 560 mg/kg/day, 565 mg/kg/day, 570 mg/kg/day, 575 mg/kg/day, 580 mg/kg/day, 585 mg/kg/day, 590 mg/kg/day, 595 mg/kg/day, 600 mg/kg/day, 605 mg/kg/day, 610 mg/kg/day, 615 mg/kg/day, 620 mg/kg/day, 625 mg/kg/day, 630 mg/kg/day, 635 mg/kg/day, 640 mg/kg/day, 645 mg/kg/day, 650 mg/kg/day, 655 mg/kg/day, 660 mg/kg/day, 665 mg/kg/day, 670 mg/kg/day, 675 mg/kg/day, 680 mg/kg/day, 685 mg/kg/day, 690 mg/kg/day, 695 mg/kg/day, 700 mg/kg/day, 705 mg/kg/day, 710 mg/kg/day, 715 mg/kg/day, 720 mg/kg/day, 725 mg/kg/day, 730 mg/kg/day, 735 mg/kg/day, 740 mg/kg/day, 745 mg/kg/day, 750 mg/kg/day, 755 mg/kg/day, 760 mg/kg/day, 765 mg/kg/day, 770 mg/kg/day, 775 mg/kg/day, 780 mg/kg/day, 785 mg/kg/day, 790 mg/kg/day, 795 mg/kg/day, 800 mg/kg/day, 805 mg/kg/day, 810 mg/kg/day, 815 mg/kg/day, 820 mg/kg/day, 825 mg/kg/day, 830 mg/kg/day, 835 mg/kg/day, 840 mg/kg/day, 845 mg/kg/day, 850 mg/kg/day, 855 mg/kg/day, 860 mg/kg/day, 865 mg/kg/day, 870 mg/kg/day, 875 mg/kg/day, 880 mg/kg/day, 885 mg/kg/day, 890 mg/kg/day, 895 mg/kg/day, 900 mg/kg/day, 905 mg/kg/day, 910 mg/kg/day, 915 mg/kg/day, 920 mg/kg/day, 925 mg/kg/day, 930 mg/kg/day, 935 mg/kg/day, 940 mg/kg/day, 945 mg/kg/day, 950 mg/kg/day, 955 mg/kg/day, 960 mg/kg/day, 965 mg/kg/day, 970 mg/kg/day, 975 mg/kg/day, 980 mg/kg/day, 985 mg/kg/day, 990 mg/kg/day, 995 mg/kg/day, or 1,000 mg/kg/day).

In some embodiments, the third composition is administered to the subject in an amount of from about 200 mg/kg/day to about 800 mg/kg/day (e.g., about 200 mg/kg/day, 205 mg/kg/day, 210 mg/kg/day, 215 mg/kg/day, 220 mg/kg/day, 225 mg/kg/day, 230 mg/kg/day, 235 mg/kg/day, 240 mg/kg/day, 245 mg/kg/day, 250 mg/kg/day, 255 mg/kg/day, 260 mg/kg/day, 265 mg/kg/day, 270 mg/kg/day, 275 mg/kg/day, 280 mg/kg/day, 285 mg/kg/day, 290 mg/kg/day, 295 mg/kg/day, 300 mg/kg/day, 305 mg/kg/day, 310 mg/kg/day, 315 mg/kg/day, 320 mg/kg/day, 325 mg/kg/day, 330 mg/kg/day, 335 mg/kg/day, 340 mg/kg/day, 345 mg/kg/day, 350 mg/kg/day, 355 mg/kg/day, 360 mg/kg/day, 365 mg/kg/day, 370 mg/kg/day, 375 mg/kg/day, 380 mg/kg/day, 385 mg/kg/day, 390 mg/kg/day, 395 mg/kg/day, 400 mg/kg/day, 405 mg/kg/day, 410 mg/kg/day, 415 mg/kg/day, 420 mg/kg/day, 425 mg/kg/day, 430 mg/kg/day, 435 mg/kg/day, 440 mg/kg/day, 445 mg/kg/day, 450 mg/kg/day, 455 mg/kg/day, 460 mg/kg/day, 465 mg/kg/day, 470 mg/kg/day, 475 mg/kg/day, 480 mg/kg/day, 485 mg/kg/day, 490 mg/kg/day, 495 mg/kg/day, 500 mg/kg/day, 505 mg/kg/day, 510 mg/kg/day, 515 mg/kg/day, 520 mg/kg/day, 525 mg/kg/day, 530 mg/kg/day, 535 mg/kg/day, 540 mg/kg/day, 545 mg/kg/day, 550 mg/kg/day, 555 mg/kg/day, 560 mg/kg/day, 565 mg/kg/day, 570 mg/kg/day, 575 mg/kg/day, 580 mg/kg/day, 585 mg/kg/day, 590 mg/kg/day, 595 mg/kg/day, 600 mg/kg/day, 605 mg/kg/day, 610 mg/kg/day, 615 mg/kg/day, 620 mg/kg/day, 625 mg/kg/day, 630 mg/kg/day, 635 mg/kg/day, 640 mg/kg/day, 645 mg/kg/day, 650 mg/kg/day, 655 mg/kg/day, 660 mg/kg/day, 665 mg/kg/day, 670 mg/kg/day, 675 mg/kg/day, 680 mg/kg/day, 685 mg/kg/day, 690 mg/kg/day, 695 mg/kg/day, 700 mg/kg/day, 705 mg/kg/day, 710 mg/kg/day, 715 mg/kg/day, 720 mg/kg/day, 725 mg/kg/day, 730 mg/kg/day, 735 mg/kg/day, 740 mg/kg/day, 745 mg/kg/day, 750 mg/kg/day, 755 mg/kg/day, 760 mg/kg/day, 765 mg/kg/day, 770 mg/kg/day, 775 mg/kg/day, 780 mg/kg/day, 785 mg/kg/day, 790 mg/kg/day, 795 mg/kg/day, or 800 mg/kg/day).

In some embodiments, the third composition is administered to the subject in an amount of from about 250 mg/kg/day to about 400 mg/kg/day (e.g., about 250 mg/kg/day, 255 mg/kg/day, 260 mg/kg/day, 265 mg/kg/day, 270 mg/kg/day, 275 mg/kg/day, 280 mg/kg/day, 285 mg/kg/day, 290 mg/kg/day, 295 mg/kg/day, 300 mg/kg/day, 305 mg/kg/day, 310 mg/kg/day, 315 mg/kg/day, 320 mg/kg/day, 325 mg/kg/day, 330 mg/kg/day, 335 mg/kg/day, 340 mg/kg/day, 345 mg/kg/day, 350 mg/kg/day, 355 mg/kg/day, 360 mg/kg/day, 365 mg/kg/day, 370 mg/kg/day, 375 mg/kg/day, 380 mg/kg/day, 385 mg/kg/day, 390 mg/kg/day, 395 mg/kg/day, or 400 mg/kg/day).

In some embodiments, the third composition is administered to the subject once daily, twice daily, three times daily, four times daily, five times daily, or six times daily. The third composition may be administered to the subject orally in admixture with cow's milk, human breast milk, infant formula, and/or water.

In some embodiments, the first composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the second composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the third composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the subject is a mammal, such as a human. In some embodiments, the subject is a pediatric human subject, such as a human subject of from about 1 month to about 12 years of age (e.g., a human subject of from about 1 month to about 1 year, 2 years, 3 years, 4 years, 5 years, 6 years, 7 years, 8 years, 9 years, 10 years, 11 years, or 12 years of age).

In a further aspect, the disclosure features a composition containing a transgene encoding TK2, dGK, TP, p53R2, SUCLA2, SUCLG1, MPV17, and/or POLG.

In some embodiments of the preceding aspect, the transgene encodes TK2. The TK2 may have an amino acid sequence that is at least 85% identical to the amino acid sequence of SEQ ID NO: 1 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 90% identical to the amino acid sequence of SEQ ID NO: 1 (e.g., an amino acid sequence that is 90%, 91%, 92%, 93%, 94%, 95%, 96%, 97%, 98%, 99%, or 100% identical to the amino acid sequence of SEQ ID NO: 1). In some embodiments, the TK2 has an amino acid sequence that is at least 95% identical to the amino acid sequence of SEQ ID NO: 1 (e.g., an amino acid sequence that is 95%, 96%, 97%, 98%, 99%, or 100% identical to the amino acid sequence of SEQ ID NO: 1). In some embodiments, the TK2 has an amino acid sequence that differs from SEQ ID NO: 1 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TK2 has an amino acid sequence that differs from SEQ ID NO: 1 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding TK2 has a nucleic acid sequence that is at least 70% identical to the nucleic acid sequence of SEQ ID NO: 2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments of the preceding aspect, the transgene encoding TK2 has a nucleic acid sequence that is at least 85% identical to the nucleic acid sequence of SEQ ID NO: 2 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 90% identical to the nucleic acid sequence of SEQ ID NO: 2 (e.g., a nucleic acid sequence that is 90%, 91%, 92%, 93%, 94%, 95%, 96%, 97%, 98%, 99%, or 100% identical to the nucleic acid sequence of SEQ ID NO: 2). In some embodiments, the transgene encoding TK2 has a nucleic acid sequence that is at least 95% identical to the nucleic acid sequence of SEQ ID NO: 2 (e.g., a nucleic acid sequence that is 95%, 96%, 97%, 98%, 99%, or 100% identical to the nucleic acid sequence of SEQ ID NO: 2). In some embodiments, the transgene encoding TK2 is codon optimized to increase efficiency. In some embodiments, the nucleic acid of the transgene encoding TK2 is at least at least 70% identical to coding sequence of the transgene that encodes TK2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TK2 has the nucleic acid sequence of SEQ ID NO: 17.

In some embodiments of the preceding aspect, the transgene encodes dGK. The dGK may have an amino acid sequence that is at least 85% identical to the amino acid sequence of SEQ ID NO: 3 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 90% identical to the amino acid sequence of SEQ ID NO: 3 (e.g., an amino acid sequence that is 90%, 91%, 92%, 93%, 94%, 95%, 96%, 97%, 98%, 99%, or 100% identical to the amino acid sequence of SEQ ID NO: 3). In some embodiments, the dGK has an amino acid sequence that is at least 95% identical to the amino acid sequence of SEQ ID NO: 3 (e.g., an amino acid sequence that is 95%, 96%, 97%, 98%, 99%, or 100% identical to the amino acid sequence of SEQ ID NO: 3). In some embodiments, the dGK has an amino acid sequence that differs from SEQ ID NO: 3 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the dGK has an amino acid sequence that differs from SEQ ID NO: 3 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding dGK has a nucleic acid sequence that is at least 70% identical to the nucleic acid sequence of SEQ ID NO: 4 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments of the preceding aspect, the transgene encoding dGK has a nucleic acid sequence that is at least 85% identical to the nucleic acid sequence of SEQ ID NO: 4 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 90% identical to the nucleic acid sequence of SEQ ID NO: 4 (e.g., a nucleic acid sequence that is 90%, 91%, 92%, 93%, 94%, 95%, 96%, 97%, 98%, 99%, or 100% identical to the nucleic acid sequence of SEQ ID NO: 4). In some embodiments, the transgene encoding dGK has a nucleic acid sequence that is at least 95% identical to the nucleic acid sequence of SEQ ID NO: 4 (e.g., a nucleic acid sequence that is 95%, 96%, 97%, 98%, 99%, or 100% identical to the nucleic acid sequence of SEQ ID NO: 4). In some embodiments, the transgene encoding dGK is codon optimized to increase efficiency. In some embodiments, the nucleic acid of the transgene encoding dGK is at least at least 70% identical to coding sequence of the transgene that encodes dGK (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the 30 coding sequence). In some embodiments, the nucleic acid sequence of the transgene encoding dGK has the nucleic acid sequence of SEQ ID NO: 18.

In some embodiments of the preceding aspect, the transgene encodes TP. The TP may have an amino acid sequence that is at least 85% identical to the amino acid sequence of SEQ ID NO: 5 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 90% identical to the amino acid sequence of SEQ ID NO: 5 (e.g., an amino acid sequence that is 90%, 91%, 92%, 93%, 94%, 95%, 96%, 97%, 98%, 99%, or 100% identical to the amino acid sequence of SEQ ID NO: 5). In some embodiments, the TP has an amino acid sequence that is at least 95% identical to the amino acid sequence of SEQ ID NO: 5 (e.g., an amino acid sequence that is 95%, 96%, 97%, 98%, 99%, or 100% identical to the amino acid sequence of SEQ ID NO: 5). In some embodiments, the TP has an amino acid sequence that differs from SEQ ID NO: 5 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the TP has an amino acid sequence that differs from SEQ ID NO: 5 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding TP has a nucleic acid sequence that is at least 70% identical to the nucleic acid sequence of SEQ ID NO: 6 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments of the preceding aspect, the transgene encoding TP has a nucleic acid sequence that is at least 85% identical to the nucleic acid sequence of SEQ ID NO: 6 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 90% identical to the nucleic acid sequence of SEQ ID NO: 6 (e.g., a nucleic acid sequence that is 90%, 91%, 92%, 93%, 94%, 95%, 96%, 97%, 98%, 99%, or 100% identical to the nucleic acid sequence of SEQ ID NO: 6). In some embodiments, the transgene encoding TP has a nucleic acid sequence that is at least 95% identical to the nucleic acid sequence of SEQ ID NO: 6 (e.g., a nucleic acid sequence that is 95%, 96%, 97%, 98%, 99%, or 100% identical to the nucleic acid sequence of SEQ ID NO: 6). In some embodiments, the transgene encoding TP is codon optimized to increase efficiency. In some embodiments, the nucleic acid of the transgene encoding TP is at least at least 70% identical to coding sequence of the transgene that encodes TP (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding TP has the nucleic acid sequence of SEQ ID NO: 19.

In some embodiments of the preceding aspect, the transgene encodes p53R2. The p53R2 may have an amino acid sequence that is at least 85% identical to the amino acid sequence of SEQ ID NO: 7 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 90% identical to the amino acid sequence of SEQ ID NO: 7 (e.g., an amino acid sequence that is 90%, 91%, 92%, 93%, 94%, 95%, 96%, 97%, 98%, 99%, or 100% identical to the amino acid sequence of SEQ ID NO: 7). In some embodiments, the p53R2 has an amino acid sequence that is at least 95% identical to the amino acid sequence of SEQ ID NO: 7 (e.g., an amino acid sequence that is 95%, 96%, 97%, 98%, 99%, or 100% identical to the amino acid sequence of SEQ ID NO: 7). In some embodiments, the p53R2 has an amino acid sequence that differs from SEQ ID NO: 7 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the p53R2 has an amino acid sequence that differs from SEQ ID NO: 7 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding p53R2 has a nucleic acid sequence that is at least 70% identical to the nucleic acid sequence of SEQ ID NO: 8 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments of the preceding aspect, the transgene encoding p53R2 has a nucleic acid sequence that is at least 85% identical to the nucleic acid sequence of SEQ ID NO: 8 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 90% identical to the nucleic acid sequence of SEQ ID NO: 8 (e.g., a nucleic acid sequence that is 90%, 91%, 92%, 93%, 94%, 95%, 96%, 97%, 98%, 99%, or 100% identical to the nucleic acid sequence of SEQ ID NO: 8). In some embodiments, the transgene encoding p53R2 has a nucleic acid sequence that is at least 95% identical to the nucleic acid sequence of SEQ ID NO: 8 (e.g., a nucleic acid sequence that is 95%, 96%, 97%, 98%, 99%, or 100% identical to the nucleic acid sequence of SEQ ID NO: 8). In some embodiments, the transgene encoding p53R2 is codon optimized to increase efficiency. In some embodiments, the nucleic acid of the transgene encoding p53R2 is at least at least 70% identical to coding sequence of the transgene that encodes p53R2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding p53R2 has the nucleic acid sequence of SEQ ID NO: 20.

In some embodiments of the preceding aspect, the transgene encodes SUCLA2. The SUCLA2 may have an amino acid sequence that is at least 85% identical to the amino acid sequence of SEQ ID NO: 9 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 90% identical to the amino acid sequence of SEQ ID NO: 9 (e.g., an amino acid sequence that is 90%, 91%, 92%, 93%, 94%, 95%, 96%, 97%, 98%, 99%, or 100% identical to the amino acid sequence of SEQ ID NO: 9). In some embodiments, the SUCLA2 has an amino acid sequence that is at least 95% identical to the amino acid sequence of SEQ ID NO: 9 (e.g., an amino acid sequence that is 95%, 96%, 97%, 98%, 99%, or 100% identical to the amino acid sequence of SEQ ID NO: 9). In some embodiments, the SUCLA2 has an amino acid sequence that differs from SEQ ID NO: 9 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLA2 has an amino acid sequence that differs from SEQ ID NO: 9 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding SUCLA2 has a nucleic acid sequence that is at least 70% identical to the nucleic acid sequence of SEQ ID NO: 10 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments of the preceding aspect, the transgene encoding SUCLA2 has a nucleic acid sequence that is at least 85% identical to the nucleic acid sequence of SEQ ID NO: 10 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 90% identical to the nucleic acid sequence of SEQ ID NO: 10 (e.g., a nucleic acid sequence that is 90%, 91%, 92%, 93%, 94%, 95%, 96%, 97%, 98%, 99%, or 100% identical to the nucleic acid sequence of SEQ ID NO: 10). In some embodiments, the transgene encoding SUCLA2 has a nucleic acid sequence that is at least 95% identical to the nucleic acid sequence of SEQ ID NO: 10 (e.g., a nucleic acid sequence that is 95%, 96%, 97%, 98%, 99%, or 100% identical to the nucleic acid sequence of SEQ ID NO: 10). In some embodiments, the transgene encoding SUCLA2 is codon optimized to increase efficiency. In some embodiments, the nucleic acid of the transgene encoding SUCLA2 is at least at least 70% identical to coding sequence of the transgene that encodes SUCLA2 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding SUCLA2 has the nucleic acid sequence of SEQ ID NO: 21.

In some embodiments of the preceding aspect, the transgene encodes SUCLG1. The SUCLG1 may have an amino acid sequence that is at least 85% identical to the amino acid sequence of SEQ ID NO: 11 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 90% identical to the amino acid sequence of SEQ ID NO: 11 (e.g., an amino acid sequence that is 90%, 91%, 92%, 93%, 94%, 95%, 96%, 97%, 98%, 99%, or 100% identical to the amino acid sequence of SEQ ID NO: 11). In some embodiments, the SUCLG1 has an amino acid sequence that is at least 95% identical to the amino acid sequence of SEQ ID NO: 11 (e.g., an amino acid sequence that is 95%, 96%, 97%, 98%, 99%, or 100% identical to the amino acid sequence of SEQ ID NO: 11). In some embodiments, the SUCLG1 has an amino acid sequence that differs from SEQ ID NO: 11 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the SUCLG1 has an amino acid sequence that differs from SEQ ID NO: 11 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding SUCLG1 has a nucleic acid sequence that is at least 70% identical to the nucleic acid sequence of SEQ ID NO: 12 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments of the preceding aspect, the transgene encoding SUCLG1 has a nucleic acid sequence that is at least 85% identical to the nucleic acid sequence of SEQ ID NO: 12 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 90% identical to the nucleic acid sequence of SEQ ID NO: 12 (e.g., a nucleic acid sequence that is 90%, 91%, 92%, 93%, 94%, 95%, 96%, 97%, 98%, 99%, or 100% identical to the nucleic acid sequence of SEQ ID NO: 12). In some embodiments, the transgene encoding SUCLG1 has a nucleic acid sequence that is at least 95% identical to the nucleic acid sequence of SEQ ID NO: 12 (e.g., a nucleic acid sequence that is 95%, 96%, 97%, 98%, 99%, or 100% identical to the nucleic acid sequence of SEQ ID NO: 12). In some embodiments, the transgene encoding SUCLG1 is codon optimized to increase efficiency. In some embodiments, the nucleic acid of the transgene encoding SUCLG1 is at least at least 70% identical to coding sequence of the transgene that encodes SUCLG1 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding SUCLG1 has the nucleic acid sequence of SEQ ID NO: 22.

In some embodiments of the preceding aspect, the transgene encodes MPV17. The MPV17 may have an amino acid sequence that is at least 85% identical to the amino acid sequence of SEQ ID NO: 13 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 90% identical to the amino acid sequence of SEQ ID NO: 13 (e.g., an amino acid sequence that is 90%, 91%, 92%, 93%, 94%, 95%, 96%, 97%, 98%, 99%, or 100% identical to the amino acid sequence of SEQ ID NO: 13). In some embodiments, the MPV17 has an amino acid sequence that is at least 95% identical to the amino acid sequence of SEQ ID NO: 13 (e.g., an amino acid sequence that is 95%, 96%, 97%, 98%, 99%, or 100% identical to the amino acid sequence of SEQ ID NO: 13). In some embodiments, the MPV17 has an amino acid sequence that differs from SEQ ID NO: 13 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the MPV17 has an amino acid sequence that differs from SEQ ID NO: 13 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding MPV17 has a nucleic acid sequence that is at least 70% identical to the nucleic acid sequence of SEQ ID NO: 14 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments of the preceding aspect, the transgene encoding MPV17 has a nucleic acid sequence that is at least 85% identical to the nucleic acid sequence of SEQ ID NO: 14 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 90% identical to the nucleic acid sequence of SEQ ID NO: 14 (e.g., a nucleic acid sequence that is 90%, 91%, 92%, 93%, 94%, 95%, 96%, 97%, 98%, 99%, or 100% identical to the nucleic acid sequence of SEQ ID NO: 14). In some embodiments, the transgene encoding MPV17 has a nucleic acid sequence that is at least 95% identical to the nucleic acid sequence of SEQ ID NO: 14 (e.g., a nucleic acid sequence that is 95%, 96%, 97%, 98%, 99%, or 100% identical to the nucleic acid sequence of SEQ ID NO: 14). In some embodiments, the transgene encoding MPV17 is codon optimized to increase efficiency. In some embodiments, the nucleic acid of the transgene encoding MPV17 is at least at least 70% identical to coding sequence of the transgene that encodes MPV17 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding MPV17 has the nucleic acid sequence of SEQ ID NO: 23.

In some embodiments of the preceding aspect, the transgene encodes POLG. The POLG may have an amino acid sequence that is at least 85% identical to the amino acid sequence of SEQ ID NO: 15 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 90% identical to the amino acid sequence of SEQ ID NO: 15 (e.g., an amino acid sequence that is 90%, 91%, 92%, 93%, 94%, 95%, 96%, 97%, 98%, 99%, or 100% identical to the amino acid sequence of SEQ ID NO: 15). In some embodiments, the POLG has an amino acid sequence that is at least 95% identical to the amino acid sequence of SEQ ID NO: 15 (e.g., an amino acid sequence that is 95%, 96%, 97%, 98%, 99%, or 100% identical to the amino acid sequence of SEQ ID NO: 15). In some embodiments, the POLG has an amino acid sequence that differs from SEQ ID NO: 15 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions). In some embodiments, the POLG has an amino acid sequence that differs from SEQ ID NO: 15 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11,12, 13, 14, 15, 16, 17, 18, 19, 20, 21, 22, 23, 24, 25, or more, conservative amino acid substitutions).

In some embodiments of the preceding aspect, the transgene encoding POLG has a nucleic acid sequence that is at least 70% identical to the nucleic acid sequence of SEQ ID NO: 16 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments of the preceding aspect, the transgene encoding POLG has a nucleic acid sequence that is at least 85% identical to the nucleic acid sequence of SEQ ID NO: 16 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 90% identical to the nucleic acid sequence of SEQ ID NO: 16 (e.g., a nucleic acid sequence that is 90%, 91%, 92%, 93%, 94%, 95%, 96%, 97%, 98%, 99%, or 100% identical to the nucleic acid sequence of SEQ ID NO: 16). In some embodiments, the transgene encoding POLG has a nucleic acid sequence that is at least 95% identical to the nucleic acid sequence of SEQ ID NO: 16 (e.g., a nucleic acid sequence that is 95%, 96%, 97%, 98%, 99%, or 100% identical to the nucleic acid sequence of SEQ ID NO: 16). In some embodiments, the transgene encoding POLG is codon optimized to increase efficiency. In some embodiments, the nucleic acid of the transgene encoding POLG is at least at least 70% identical to coding sequence of the transgene that encodes POLG (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of the coding sequence). In some embodiments, the nucleic acid sequence of the transgene encoding POLG has the nucleic acid sequence of SEQ ID NO: 24.

In some embodiments of the preceding aspect, the composition is a vector, such as a viral vector. The viral vector may be, for example, an AAV, adenovirus, lentivirus, retrovirus, poxvirus, baculovirus, herpes simplex virus, vaccinia virus, or a synthetic virus (e.g., a chimeric virus, mosaic virus, or pseudotyped virus, and/or a virus that contains a foreign protein, synthetic polymer, nanoparticle, or small molecule).

In some embodiments of the preceding aspect, the viral vector is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments of the preceding aspect, the viral vector is a pseudotyped AAV, containing ITRs from one AAV serotype and capsid proteins from a different AAV serotype. 5 In some embodiments, the pseudotyped AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments of the preceding aspect, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAVrh74, AAVrh.8, or AAVrh.10.

In some embodiments of the preceding aspect, the composition is a liposome, vesicle, synthetic vesicle, exosome, synthetic exosome, dendrimer, or nanoparticle.

In some embodiments of the preceding aspect, the transgene is operably linked to a promoter that induces expression of the transgene in a muscle cell. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments of the preceding aspect, the transgene is operably linked to an enhancer that induces expression of the transgene in a muscle cell. Exemplary enhancers that may be used in conjunction with the compositions and methods of the disclosure are a CMV enhancer, a myocyte enhancer factor 2 (MEF2) enhancer, and a MyoD enhancer.

In another aspect, the disclosure features a kit containing the composition of the preceding aspect. The kit may further contain a package insert, such as a package insert instructing a user of the kit to administer the composition to a subject in accordance with the method of any of the above aspects or embodiments of the disclosure. The kit may further contain a pharmacological agent selected from the group consisting of dC, dT, dA, dG, dCMP, TMP, dAMP, dGMP, and mixtures thereof.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

In the drawings, certain abbreviations are used. “WT” means wild-type mice which are Tk2+. “Het” means mice heterozygous for the TK2 gene. “Mut” means Tk2^{-/-} mice. “P #” means post-natal day #. “AAV2-hTK2” refers to an AAV2 vector containing a human TK2 transgene. “AAV9-hTK2” refers to an AAV9 vector containing ahuman TK2 transgene. “dC” refers to deoxycytidine. “dT” refers to deoxythymidine. “IV” refers to intravenous administration. “vC” refers to a quantity of vector genome copies.

FIG. 1 is a schematic of a recombinant adeno-associated virus (AAV) vector comprising human TK2 cDNA.

FIG. 2 shows the survival curves of the following mice (curves from left to right): Tk2^{-/-} mice untreated (n=13); Tk2^{-/-} mice administered milk only (n=6); Tk2^{-/-} mice treated with AAV9-hTK2 at 4.2×10¹⁰ vc by IV at postnatal day 1 (n=5); Tk2^{-/-} mice treated with 520 mg of dC+dT (n=11); and Tk2^{-/-} mice treated with AAV9-hTK2 at 4.2×10¹¹ vc by IV at postnatal day 1 (n=10). Survival of untreated Tk2+(wild-type) is shown by the top horizontal line.

FIG. 3 shows graphs of weight versus time in the following male and female mice: Tk2⁺ treated with 520 mg of dC+dT; Tk2^{-/-} mice treated with 520 mg of dC+dT; Tk2⁺ treated AAV9-hTK2; Tk2^{-/-} mice treated with AAV9-hTK2 at 4.2×10¹¹ vc by IV at postnatal day 1; and Tk2^{-/-} mice treated with AAV9-hTK2 at 4.2×10¹⁰ vc by IV at postnatal day 1.

FIG. 4 shows graphs of the result of grip strength test of the fore limbs (bar test) and both fore and hind limbs (grid test) normalized by mouse weight at postnatal day 29 and postnatal day 60 of male and female mice including untreated Tk2⁺, Tk2⁺ treated with AAV9-hTK2 and Tk2^{-/-} mice treated with AAV9-hTK2.

FIG. 5 shows graphs of the results of mice motor function as measured by accelerating rotarod performance test at postnatal day 29 and postnatal day 60 of male and female mice including Tk2⁺ treated with AAV9-hTK2 and Tk2^{-/-} mice treated with AAV9-hTK2.

FIG. 6 shows graphs of the activity of TK2 enzyme measured as pmol/min/mg of protein at postnatal day 29 in brain, liver, muscle and kidney tissue of the following mice: untreated Tk2⁺; Tk2⁺ treated with AAV9-hTK2 at 4.2×10¹¹ vc; Tk2^{-/-} mice treated with AAV9-hTK2 at 4.2×10¹¹ vc; Tk2⁺ treated with 520 mg of dC+dT; and Tk2^{-/-} mice treated with 520 mg of dC+dT.

FIG. 7 shows graphs of mRNA expression of human TK2 measure as a percent of mouse TK2 mRNA in brain, liver, muscle and kidney tissue of Tk2⁺ treated with AAV9-hTK2 at one month, two months and six months.

FIG. 8 shows graphs of showing mtDNA copy number (mtDNA/nDNA as a percent of untreated Tk2+) at postnatal day 29 in brain, liver, kidney, heart, muscle, and intestine tissue of the following mice: untreated Tk2⁺; Tk2⁺ treated with AAV9-hTK2; mice which are heterozygous for the Tk2 gene treated with AAV9-hTK2; Tk2^{-/-} mice treated with AAV9-hTK2; and Tk2^{-/-} mice treated with 520 mg of dC+dT.

FIG. 9 shows graphs of showing mtDNA copy number (mtDNA/nDNA as a percent of untreated Tk2+) at postnatal day 60 in brain, liver, kidney, heart, muscle, and intestine tissue of the following mice: untreated Tk2⁺; Tk2⁺ treated with AAV9-hTK2; mice which are heterozygous for the Tk2 gene treated with AAV9-hTK2; and Tk2^{-/-} mice treated with AAV9-hTK2.

FIG. 10 shows graphs of respiratory chain enzyme activity at postnatal day 29 in brains of the following mice: untreated Tk2⁺; Tk2⁺ treated with AAV9-hTK2; mice which are heterozygous for the Tk2 gene treated with AAV9-hTK2; Tk2^{-/-} mice treated with AAV9-hTK2; and Tk2^{-/-} mice treated with 520 mg of dC+dT.

FIG. 11 shows graphs of respiratory chain enzyme levels (oxphos steady-state levels normalized to vinculin) at postnatal day 29 in brains of the following mice: untreated Tk2⁺; Tk2⁺ treated with AAV9-hTK2; Tk2^{-/-} mice treated with AAV9-hTK2; and Tk2^{-/-} mice treated with 520 mg of dC+dT.

FIG. 12 shows images of stained (H & E) kidneys of AAV9-hTK2 treated mutant and wild-type mice at postnatal day 96.

FIG. 13 shows graphs of creatinine and BUN index in AAV9-hTK2 treated mutant and wild-type mice in a survival cohort and a postnatal day 60 cohort.

FIG. 14 shows the survival curve of the following mice (curves from left to right): Tk2^{-/-} mice untreated (n=13); Tk2^{-/-} mice treated with AAV9-hTK2 at 4.2×10¹⁰ vc by IV at postnatal day 1 (n=5); Tk2^{-/-} mice treated with 520 mg of dC+dT (n=11); Tk2^{-/-} mice treated with AAV9-hTK2 at 4.2×10¹¹ vc by IV at postnatal day 1 (n=10); mice co-treated with AAV9-hTK2 at 2.1×10¹¹ at day 1 and AAV2-hTK2 at 1.05×10¹¹ at day 29 (n=7); and mice under co-treatment with AAV9-hTK2 at day 1, AAV2-hTK2 at day 29 and supplemented with 520 mg/kg/day of oral dC+dT from day 21 (n=7). Survival of untreated Tk2⁺ (wild-type) is shown by the top horizontal line.

FIG. 15 shows weight versus time in the following male mice: Tk2⁺ untreated; Tk2⁺ treated with 520 mg of dC+dT; Tk2^{-/-} mice treated with 520 mg of dC+dT; Tk2⁺ treated AAV9-hTK2 at 4.2×10¹¹ vc by IV at postnatal day 1; Tk2^{-/-} mice treated with AAV9-hTK2 at 4.2×10¹¹ vc by IV at postnatal day 1 (“AAV9”); Tk2^{-/-} mice treated with AAV9-hTK2 at 4.2×10¹⁰ vc by IV at postnatal day 1; Tk2 mice treated with AAV9-hTK2 at 2.1×10¹¹ vc by IV at postnatal day 1 and AAV2-hTK2 at 1.05×10¹¹ vc by IV at postnatal day 29 (“AAV9+AAV2”); and Tk2^{-/-} mice treated with AAV9-hTK2 at 2.1×10¹¹ vc by IV at postnatal day 1, AAV2-hTK2 at 1.05×10¹¹ vc by IV at postnatal day 29 and 520 mg/kg/day of oral dC+dT from day 21 (“AAV9+AAV2+dCdT”).

FIG. 16 shows weight versus time in the following female mice: Tk2+ untreated; Tk2+ treated with 520 mg of dC+dT; Tk2^{-/-} mice treated with 520 mg of dC+dT; Tk2⁺ treated AAV9-hTK2 at 4.2×10¹¹ vc by IV at postnatal day 1; Tk2^{-/-} mice treated with AAV9-hTK2 at 4.2×10¹¹ vc by IV at postnatal day 1 (“AAV9”); Tk2^{-/-} mice treated with AAV9-hTK2 at 4.2×10¹⁰ vc by IV at postnatal day 1; Tk2^{-/-} mice treated with AAV9-hTK2 at 2.1×10¹¹ vc by IV at postnatal day 1 and AAV2-hTK2 at 1.05×10¹¹ vc by IV at postnatal day 29 (“AAV9+AAV2”); and Tk2^{-/-} mice treated with AAV9-hTK2 at 2.1×10¹¹ vc by IV at postnatal day 1, AAV2-hTK2 at 1.05×10¹¹ vc by IV at postnatal day 29 and 520 mg/kg/day of oral dC+dT from day 21 (AAV9+AAV2+dCdT”).

FIG. 17 shows graphs of the result of grip strength test of the fore limbs (bar test) and both fore and hind limbs (grid test) normalized by mouse weight at postnatal day 60 of male and female mice including untreated Tk2+, Tk2⁺ treated with AAV9-hTK2, Tk2^{-/-} mice treated with AAV9-hTK2, Tk2^{-/-} mice treated with AAV9-hTK2 and AAV2-hTK2, and Tk2^{-/-} mice treated with AAV9-hTK2, AAV2-hTK2 and dC+dT.

FIG. 18 shows graphs of the result of grip strength test of the fore limbs (bar test) and both fore and hind limbs (grid test) normalized by mouse weight at postnatal day 90 of male and female mice including untreated Tk2+, Tk2⁺ treated with AAV9-hTK2, Tk2^{-/-} mice treated with AAV9-hTK2, Tk2^{-/-} mice treated with AAV9-hTK2 and AAV2-hTK2, and Tk2^{-/-} mice treated with AAV9-hTK2, AAV2-hTK2 and dC+dT.

FIG. 19 shows graphs of the results of mice motor function as measured by accelerating rotarod performance test at postnatal day 60 and postnatal day 90 of male and female mice including untreated Tk2+, Tk2⁺ treated with AAV9-hTK2, Tk2^{-/-} mice treated with AAV9-hTK2, Tk2^{-/-} mice treated with AAV9-hTK2 and AAV2-hTK2, and Tk2^{-/-} mice treated with AAV9-hTK2, AAV2-hTK2 and dC+dT.

FIG. 20 shows graphs of showing mtDNA copy number (mtDNA/nDNA as a percent of untreated Tk2+) at postnatal day 60 in brain, liver, muscle, heart, kidney, and intestine tissue of the following mice: untreated Tk2⁺; Tk2^{-/-} mice treated with AAV9-hTK2; Tk2^{-/-} mice treated with AAV9-hTK2 and AAV2-hTK2; and Tk2^{-/-} mice treated with AAV9-hTK2, AAV2-hTK2 and dC+dT.

FIG. 21 depicts the results from the protein test in a urine dip stick using urine from mice at age 21-29 days and age 60 days. All Tk2^{-/-} mice were treated with AAV9-hTK2 at day 1, while 3 out of the four Tk2^{-/-} mice were treated with AAV2-hTK2 at day 29.

FIG. 22 shows graphs of BUN index in mice at day 60 and mice in an endpoint cohort including untreated Tk2+, Tk2⁺ treated with AAV9-hTK2, Tk2^{-/-} mice treated with AAV9-hTK2, Tk2^{-/-} mice treated with AAV9-hTK2 and AAV2-hTK2, and Tk2^{-/-} mice treated with AAV9-hTK2, AAV2-hTK2 and dC+dT.

FIG. 23 shows graphs of the activity of TK2 enzyme measured as pmol/min/mg of protein at postnatal day 60 in brain, liver, muscle and kidney tissue of the following mice: untreated Tk2⁺; Tk2⁺ treated with AAV9-hTK2; Tk2^{-/-} mice treated with AAV9-hTK2; Tk2^{-/-} mice treated with AAV9-hTK2 and AAV2-hTK2; and Tk2^{-/-} mice treated with AAV9-hTK2, AAV2-hTK2 and dC+dT.

### DETAILED DESCRIPTION OF THE INVENTION

The current invention is based, at least in part, upon the surprising discovery that mitochondrial DNA depletion syndromes, including TK2 deficiency, can be treated, prevented and/or cured via gene therapy by delivery of nucleic acids encoding for a functional protein, such as a functional protein encoded by a nuclear gene. Exemplary nucleic acids that can be used in conjunction with the compositions and methods of the disclosure encode thymidine kinase 2 (TK2), deoxyguanosine kinase (dGK), thymidine phosphorylase (TP), p53 inducible small subunit of ribonucleotide reductase (p53R2), succinyl-CoA ligase ADP-forming subunit beta (SUCLA2), succinyl-CoA ligase GDP-forming subunit alpha (SUCLG1), mitochondrial inner membrane protein MPV17 (MPV17), and/or DNA polymerase subunit gamma (POLG). Such nucleic acids can be delivered by way of one or more viral vectors, such as by way of an adeno-associated virus (AAV), among others described herein. The gene therapy can be administered alone or in combination with pharmacological therapy, such as deoxynucleoside or deoxyribonucleoside monophosphate therapy. The sections that follow describe the compositions and methods of the disclosure in further detail.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the 10 specification, including examples of any terms discussed herein, is illustrative only, and in no way limits the scope and meaning of the invention or any exemplified term. Likewise, the invention is not limited to its preferred embodiments.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The terms “mutant mouse”, “TK2 knockin mouse” and “Tk2^{-/-}, refer to the homozygous 25 Tk2 H126N knock-in mutant (Tk2^{-/-}) mouse model, described in Akman, et al. 2008. The terms “wild-type mouse”, “WT”, and “Tk2+” will be used interchangeably as well. In some cases, mice which are heterozygous for the Tk2 gene (“Tk2^{-/-}) are used for comparison and would be expected to behave the same as the wild-type mice. These mice are designated “Het.” The term “subject” as used in this application refers to animals in need of therapeutic or prophylactic treatment. Subjects include mammals, such as canines, felines, rodents, bovine, equines, porcines, ovines, and primates. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is known or suspected of having a disease or disorder characterized by unbalanced nucleotide pools, mitochondrial disease, mitochondrial DNA depletion syndrome, or TK2 deficiency.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small.

The term “in need thereof” would be a subject known or suspected of having or being at risk of having a disease or disorder characterized by unbalanced nucleotide pools, mitochondrial disease, mitochondrial DNA depletion syndrome, or TK2 deficiency.

The term “agent” as used herein means a substance that produces or is capable of producing an effect and would include, but is not limited to, chemicals, pharmaceuticals, biologics, small organic molecules, antibodies, nucleic acids, peptides, and proteins.

The terms “deoxynucleoside” or “dN” as used herein means deoxythymidine or dT, deoxycytidine or dC, deoxyadenosine or dA, and deoxyguanosine or dG. The full length name and common abbreviation for each will be used interchangeably. Such deoxynucleosides also include physiologically functional derivatives of the deoxynucleosides.

The term “deoxyribonucleoside monophosphate” as used herein means thymidine-5′-monophosphate or (TMP) or 2′-deoxycytidine-5′-monophosphate (dCMP), deoxyadenosine monophosphate or dAMP, and deoxyguanosine monophosphate or dGMP. The full length name and common abbreviation for each will be used interchangeably.

The term “pharmacological agent” as used herein means deoxynucleosides or dNs and/or deoxyribonucleoside monophosphate or dNMPs, alone or in a composition or a pharmaceutical composition.

The terms “pharmacological therapy” or “pharmacological treatment” as used herein means the administration of dNs and/or dNMPs as disclosed herein and as fully described in co-owned U.S. application Ser. No. 15/082,207 (hereinafter “the '207 application”) and U.S. Ser. No. 15/736,092 (hereinafter the '092 application”).

As used herein, the term “physiologically functional derivative” refers to a compound (e.g, a drug precursor) that is transformed in vivo to yield a deoxynucleoside. The transformation may occur by various mechanisms (e.g., by metabolic or chemical processes), such as, for example, through hydrolysis in blood. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

As used herein “an adverse effect” is an unwanted reaction caused by the administration of a drug. In most cases, the administration of the gene and pharmacological therapy caused no adverse effects.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

In some aspects, the invention provides isolated adeno-associated vectors (AAVs). As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities.

Methods for obtaining recombinant AAVs having a desired capsid protein have been described (See, for example, U.S. Pat. No. 7,906,111). A number of different AAV capsid proteins have been described, for example, those disclosed in Gao, et al., J Virology 78(12):6381-6388 (June 2004); Gao, et al., Proc Natl Acad Sci USA 100(10):6081-6086 (May 13, 2003); and U.S. Pat. Nos. 7,906,111; 8,999,678. For the desired packaging of the presently described constructs and methods, the AAV9 vector and capsid, or the AAV2 vector and capsid, is preferred. However, it is noted that other suitable AAVs such as rAAVrh.8 and rAAVrh.10, or other similar vectors may be adapted for use in the present invention. Typically the methods involve culturing a host cell which contains a nucleic acid sequence encoding an AAV capsid protein or fragment thereof, a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., Fisher et al, J. Virology 70:520-532 (1993) and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

As used herein, the terms “AAV1,” “AAV2,” “AAV3,” “AAV4,” and the like refer to AAV vectors containing ITRs from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

As used herein, the term “TK2” refers to a gene encoding thymidine kinase 2, or the corresponding protein product. The terms “TK2” and “thymidine kinase 2” include wild-type forms of the TK2 gene or protein, as well as variants (e.g., splice variants, truncations, concatemers, and fusion constructs, among others) of wild-type TK2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TK2 protein (e.g., SEQ ID NO: 1), provided that the TK2 variant retains the function of a wild-type TK2. Additionally, the terms “TK2” and “thymidine kinase 2” may refer to fusion proteins, or nucleic acids encoding the same, in which the TK2 is operably linked to another polypeptide, half-life-modifying agent, or therapeutic agent. The term “TK2” may refer to the protein or the gene encoding this protein, depending upon the context, as will be appreciated by one of skill in the art.

Exemplary TK2 amino acid sequence (SEQ ID NO: 1):
MGAFCQRPSSDKEQEKEKKSVICVEGNIASGKTTCLEFFSNATDVEVLTEPVSKWRNVRGHNPLGLMY
HD
ASRWGLTLQTYVQLTMLDRHTRPQVSSVRLMERSIHSARYIFVENLYRSGKMPEVDYVVLSEWFDWIL
RN
MDVSVDLIVYLRTNPETCYQRLKKRCREEEKVIPLEYLEAIHHLHEEWLIKGSLFPMAAPVLVIEADHH
M
ERMLELFEQNRDRILTPENRKHCP
Exemplary TK2 nucleic acid sequence (SEQ ID NO: 2):
GCCAAGTTATGGGTGCGTTCTGCCAGCGTCCTAGCAGTGATAAAGAACAGGAAAAAGAGAAAAAATCAGT
GATCTGTGTCGAGGGCAATATTGCAAGTGGGAAGACGACATGCCTGGAATTCTTCTCCAACGCGACAGAC
GTCGAGGTGTTAACGGAGCCTGTGTCCAAGTGGAGAAATGTCCGTGGCCACAATCCTCTGGGCCTGATGT
ACCACGATGCCTCTCGCTGGGGTCTTACGCTACAGACTTATGTGCAGCTCACCATGCTGGACAGGCATAC
TCGTCCTCAGGTGTCATCTGTACGGTTGATGGAGAGGTCGATTCACAGCGCAAGATACATTTTTGTAGAA
AACCTGTATAGAAGTGGGAAGATGCCAGAAGTGGACTATGTAGTTCTGTCGGAATGGTTTGACTGGATCT
TGAGGAACATGGACGTGTCTGTTGATTTGATAGTTTACCTTCGGACCAATCCTGAGACTTGTTACCAGAG
GTTAAAGAAGAGATGCAGGGAAGAGGAGAAGGTCATTCCGCTGGAATACCTGGAAGCAATTCACCATCTC
CATGAGGAGTGGCTCATCAAAGGCAGCCTTTTCCCCATGGCAGCCCCTGTTCTGGTGATTGAGGCTGACC
ACCACATGGAGAGGATGTTAGAACTCTTTGAACAAAATCGGGATCGAATATTAACTCCAGAGAATCGGAA
GCATTGCCCATAGGAGGCAAAAGGTCTATGGCTCATGTCTGAAAAATGCCTGCTGCTGCCAAGTTAGCTA
TTGGGAGCAATCTGGAAAAACTTGCTCCCAGGAGGGCTTTGTGTCTGGCCAGCTTGATTTTCCTAATGGT
CTCATCTCCTTTGCTAGTGTCTTTGTCATGCGTCTCTGGCCCTCGTGGGTAAATGACAAACGGGACCAAT
GGGTTTGCCAAGCCCTTTGCTGTTCGCAGCCTCACATTCCCCCGGTGCCTCTCCCATGGCTTTGTGCTGC
TGAGTCGCTCTCATGAAGCCCTTAGGGAGAGCACCTGTTGTGTGCCTGACACCACGCTGGAGCTGTGTAC
CAATCGTCTCAGCCTTCATTAGGAGGCCGAGGTAGGAGTCTTATATCCCAGGTGAGGAATTTGAAGCTCA
GAAAGGTTGAGGGGCTCCCCAGAGGTCACACAGCCTGTGTGCAGTGGAGCTGGCACCATTCAGACTTTCA
GCCGACTCAGCAACTTTCCCTTGCCCTGGGCTGCCTCCTCCTGAGAGCTGTTCCCCACCGCCCTGCCTCT
TCCGGTTGGAGGCTCTCATGTCTCTTTGGGGAGAGCTGGCAGTGTGCGGAGCTGATAACATTTTCCCAAT
ATTGAGCAGTTCCCAAGGACAGTCAGCATTTCTAGACTTCCACAAAATTATGCTGCATTTGGCTGGAGCC
CGGTGTTCAGTGGTTTCCCTGCCCGAGGTCGCTGCAGCCCCATCTACCACATCTTCATGTGGACATTGAG
ATTCACATGCTGGCTCCTGAAGGGTGCTCAGTCTCCTTGGTGATTAAGGTCCTGCTTGAACTGCTGCCAA
CTCCATGTCAGGGAAGTCGCTTTTGGTGCCTGGCTGGTTTGCCCAGAGCCAAGCTGGGGCAAGGGGCAGC
CAGCCCTGGCTTCCAAGGCTCCCGTACTGTCTGTGTCCTTGTATAAGGAGCTTTGCTCTTGGAATTACTG
AAAGTCTGTGGCCCAAGAGAGAGACACAAGTGGCCTTAAGTCTTTTTGAAGTGTTATTTCATCCAGGGAA
ATGCCTCGAGCCATAGAGCCTGAAATCATCTTTGTTGGCTCAGAAAATACCTTAGCTTCACTCAGCTGGA
CTGCATTGAAGGCGAGGCTGCCCCTTGGATCAAGCAGAAAACAAGAGAAAGAAAGAACGTTCCCTTTGGG
GATAGTCTGGAAAGTTGGGATTTGCAAATAAAGGCTCTGGAAGCATTGCTGGTCCTGAAGCTTTGGAGGT
GGGCAGAGAGAGCTTCAAGAAGACTAGATGCAAACCCTGGAAAGGATTAAGGCTCAACTCTGGAGAAACA
GGCCACAGCCTCTCAGAGCAGCTGTTGGCTGTAAATAGAGGTAGCAAGGCCGCTCCCAGGCCCCTGTGAG
TGTGGGCACCTGTGCATGCAATGCTCCGACTCTGCAGAGGTGCCAAGTGCCCCTGCTGGGCCAGTCCCAG
AGAGTTAGGAAGTCAAGGCCTGCAACTCCTGGTTCTTCCTGTTTGGACCAGTTCTTGTGCCATTGGCAGG
ATGAGAGGCAGCAGCCAGGCGGGAGCTGTGTCTAGCAGCACCTGTAGCCCACGTGCTGCTAATTAGCTGG
AAAACTGGCGAAGGCAGAACCTTTGCTACCAGGAATCTTGACATGTGGGGTCTGTCTTTGAGAATTTGTA
AATGAACAGTCCAATATTTCCTCTCGGCTCATTTTGCACATCCATTTTTGGGGAAATGTGATTTCTCTCT
CTTTTTTTTTTTTTTTTTTTGCCTAAGGCACAATCTCAAGAGGTCCTGAGACCACGTACCCATGATTTTT
TTCTTGCTCTGTGATACCCAATAACTCCTTCACCTAAGCCCTGTTGTTGATTTTGAAGTGCTTCCTAGGC
CAGTGATTTTAGCTTCTGCCAGCTGCTTTTGCCAGTAGATTAACGTGTTTTTATTTTTCAAACTCCGTGT
TTCCTAACGTGGAGTGTATGGGTCTAAGAGAGCCTGCTGTCCTCCCTGCCTTCCACCTTGGAGAGGAGGC
TGGACGCATCAGCAGTGGCCAGGGCAGGTCGCAAAATCTCCCAGCCTAGAGACCACACCTGAAACGGCTG
AAGCCAGCTTGCACAAGGGCTGCTGTCCCTCTGCGGCAGGCAGAGCTGGTGGGGGCAGGGGTCACAGAGC
AGTCATAGACACCATGGACCAGGGCAGGAGAAGGGCAGATGGCACATGGGCACAACAGGGCCTTGTCCTT
AGAGCACTGGGGGGTCATGGCTGGGAGGGGCATGGCAGGGGCTGGCATCCCTGTAGAGCCAGAGGGGCCA
CCCAGGGCAGTGACATTCCAGATATGTTGGGCTCACCTCATCCTTGCTGTGAGACTGGAGTTCCATGGGG
ACATGAAGTCAGTACACCGCAGAGCTGCTCAGCTGCTCTACCTCTCGCTGACTTTTTTGTTGCACATATA
CATTTTCTTTCAATTAGCATTTATTTCAGCTTTTATTTAAGCTTTTTGACAGTACATGTAAAATATATGA
TTATAACCATTTAAAAATACCTTATGTACCTGGTTTTTTTTGGAAACTAGATAGAAATATATTTATCTTT
TACATAAAAGAAGTGTGTAGTGGGGTGGTCCAGGGCTTTGTGGTGGCTTAGTGGCCATCGGGGTCCCAGG
CTCTTCCCACCTTTCTCTTTTGGGTCCACCTCTTGGCTCCTGGCTTCCTTCCTCTGGGGCCTGACTGTCC
AGGATGGCAGCTGGAGCTCCTGCTCTGGGCACGGTTGGCATCAGAGCCCACTGCTCCCCATCCACTCTTT
AATCCAGATGCTGGTAATGTCCCTTTCCAAGGCATAACTAAGATAAGCTGGAAGGTTCTTACGAGGCTTT
GCTAGAGGCCCTGGGAGGTGGGGGGAAAGGCAAGAGGGCAGTGCCCACCCATAGACCGGGTCACATGACC
TGGCATCAGCGCCGTGGGGTCCTTTGGGCTTCACCTCCCTCTCCCCTCTGCCCCCACGTCATCCCACCCT
CATCCTCACCCTCACCATTCCCATCCCGTACCGTAATGTCCCTTGCAAGGCCTAACTCTGTGAGGAATGA
AACCACCTCATCCTTGTTCCAATAACATGAGTGACCGAATTTACAAACGAGTGAATGTGGACCTCTGGAA
ACATTACCCAGCTTGTTTTTACTTTTCACTTTCTCTTTCTGCCCCTTTCATTTCCGTAGGAGCCCTTTAC
CTAGATGAGAAGTGTCCCCCCGCCTGGGGAATATATCAGTCAGAACAATCTTCCTGCAGACATGCACCAT
TAGACCCGAGTGACGGTGGTGCCATTTAAACCTCAGAGCAGGTAAAAGGTGGTCCTGAAACCTGTCTACC
CACAGGGTGCTATGGAATCTGAATCACTTCTTTTTTCCTAGAGCCCTGGGGTGGGGAGCTCCCTCAAGTG
TTCACATGTGTGTGGAATGAGGAACACCCATCTCCTTGGCCCTCTCCACCCTGAAGAGTTAGTTATTAAA
ATAATTGGCAAGCTCTTGCAAATGTCAGTCATCCATTGTTCAGAATGGAATAGCAATAATACATCCCTGG
CTGCCCTGGGCTTGGCCAGGATTACTCACTGAAGGCCTCAGGGTTACTGGCACACACTTTCTTTTCCTAA
TAATCCCATCCCCTCAGCTTTCCTAAGGCTAGAGTGAATTTCGTGTTCCTTTAGTTTACATAAGATGGTG
AACTTGGCAAAAGCTATCATTAAACAGAAGCTAAGAGAAAGCCTATGTCGTGGAATCCAGAATGGGTATT
GCCATTCACTGCTGTCCACAGAAGCTGTCTTGAATTTCTTTCTGTGTCTTTTCTTTTTTTTTCTTTAAGA
CTGTTGTTTACCAGACTGGGCTCTGTGGAACACAGGTGTCCTGGGAGATGGTTAATCATTACAAAATATT
GGTAACAATCTAAAGATGCATACATAAGAGAGTGGTCAAATAAACCATTTTCCATTCA

As used herein, the term “dGK” refers to a gene encoding deoxyguanosine kinase, or the corresponding protein product. The terms “dGK” and “deoxyguanosine kinase” include wild-type forms of the dGK gene or protein, as well as variants (e.g., splice variants, truncations, concatemers, and fusion constructs, among others) of wild-type dGK proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type dGK protein (e.g., SEQ ID NO: 3), provided that the dGK variant retains the function of a wild-type dGK. Additionally, the terms “dGK” and “deoxyguanosine kinase” may refer to fusion proteins, or nucleic acids encoding the same, in which the dGK is operably linked to another polypeptide, half-life-modifying agent, or therapeutic agent. The term “dGK” may refer to the protein or the gene encoding this protein, depending upon the context, as will be appreciated by one of skill in the art.

Exemplary dGK amino acid sequence (SEQ ID NO: 3):
MAAGRLFLSRLRAPFSSMAKSPLEGVSSSRGLHAGRGPRRLSIEGNIAVG
KSTFVKLLTKTYPEWHVATEPVATWQNIQAAGTQKACTAQSLGNLLDMMY
REPARWSYTFQTFSFLSRLKVQLEPFPEKLLQARKPVQIFERLHFEALMN
IPVLVLDVNDDFSEEVTKQEDLMREVNTFVKNL
Exemplary dGK nucleic acid sequence (SEQ ID NO:
4):
AACGGTGCGCTGGAGCGAGTGAGCAGCGATACCTAGGGCGGAAGTGCTCT
CGGCGGAAGTGATCGCTGTGTGAATCGTGGGTGGGATGGCCGCGGGCCGC
CTCTTTCTAAGTCGGCTTCGAGCACCCTTCAGTTCCATGGCCAAGAGCCC
ACTCGAGGGCGTTTCCTCCTCCAGAGGCCTGCACGCGGGGCGCGGGCCCC
GAAGGCTCTCCATCGAAGGCAACATTGCTGTGGGAAAGTCCACGTTTGTG
AAGTTACTCACGAAAACTTACCCAGAATGGCACGTAGCTACAGAACCTGT
AGCAACATGGCAGAATATCCAGGCTGCTGGCACCCAAAAAGCCTGCACTG
CCCAAAGTCTTGGAAACTTGCTGGATATGATGTACCGGGAGCCAGCACGA
TGGTCCTACACATTCCAGACATTTTCCTTTTTGAGCCGCCTGAAAGTACA
GCTGGAGCCCTTCCCTGAGAAACTCTTACAGGCCAGGAAGCCAGTACAGA
TCTTTGAGAGGCTCCACTTTGAGGCTCTGATGAACATTCCAGTGCTGGTG
TTGGATGTCAATGATGATTTTTCTGAGGAAGTAACCAAACAAGAAGACCT
CATGAGAGAGGTAAACACCTTTGTAAAGAATCTGTAACCAATACCATGAA
GTTCAGGCTGTGATCTGGGCTCCCTGACTTTCTGAAGCTAGAAAAATGTT
GTGTCTCCCAACCACCTTTCCATCCCCAGCCCCTCTCATCCCTGGAGCAC
TCTGCCGCTCAAGAGCTGGTTTGTTAATTATTGTTAGACTTTGCCATTGT
TTTCTTTTGTACCTGAAGCATTTTGAAAATAAAGTTTACTTAAGTTATGC
TTGTTTTTCTAAAAAAAAAAAAAAAAAAA

As used herein, the term “p53R2” refers to a gene encoding p53 inducible subunit of 5 ribonucleotide reductase, or the corresponding protein product. The terms “p53R2” and “p53 inducible subunit of ribonucleotide reductase” include wild-type forms of the p53R2 gene or protein, as well as variants (e.g., splice variants, truncations, concatemers, and fusion constructs, among others) of wild-type p53R2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type p53R2 protein (e.g., SEQ ID NO: 5), provided that the p53R2 variant retains the function of a wild-type p53R2. Additionally, the terms “p53R2” and “p53 inducible subunit of ribonucleotide reductase” may refer to fusion 15 proteins, or nucleic acids encoding the same, in which the p53R2 is operably linked to another polypeptide, half-life-modifying agent, or therapeutic agent. The term “p53R2” may refer to the protein or the gene encoding this protein, depending upon the context, as will be appreciated by one of skill in the art.

Exemplary p53R2 amino acid sequence (SEQ ID NO: 5):
MLLLRLPPHRSHASPLDCKLQDRCRKCYSPRSGQACPPALAAAWLRRCERRGGRPRGGRRKELTLGLRPA
RCSAPGPAKDDAWRPQAGRSSSDTNESEIKSNEEPLLRKSSRRFVIFPIQYPDIWKMYKQAQASFWTAEE
VDLSKDLPHWNKLKADEKYFISHILAFFAASDGIVNENLVERFSQEVQVPEARCFYGFQILIENVHSEMY
SLLIDTYIRDPKKREFLFNAIETMPYVKKKADWALRWIADRKSTFGERVVAFAAVEGVFFSGSFAAIFWL
KKRGLMPGLTFSNELISRDEGLHCDFACLMFQYLVNKPSEERVREIIVDAVKIEQEFLTEALPVGLIGMN
CILMKQYIEFVADRLLVELGFSKVFQAENPFDFMENISLEGKTNFFEKRVSEYQRFAVNAETTDNVFTLD
ADF
Exemplary p53R2 nucleic acid sequence (SEQ ID NO: 6):
ATGAGGTAAATGTTGCTGTTGCGTCTTCCCCCTCACCGCAGTCACGCCAGCCCGTTAGATTGCAAGTTGC
AGGACCGCTGTAGGAAATGTTATTCGCCGCGGTCAGGACAGGCCTGTCCGCCCGCCCTCGCCGCAGCCTG
GCTTCGTCGTTGCGAGCGCCGGGGAGGCCGTCCCCGGGGAGGGCGGAGGAAGGAGCTGACTTTGGGTTTG
CGTCCCGCTCGCTGCTCTGCCCCGGGGCCAGCCAAGGACGACGCTTGGAGGCCTCAGGCCGGGAGATCAT
CTTCAGACACCAACGAAAGTGAAATAAAGTCAAATGAAGAGCCACTCCTAAGAAAGAGTTCTCGCCGGTT
TGTCATCTTTCCAATCCAGTACCCTGATATTTGGAAAATGTATAAACAGGCACAGGCTTCCTTCTGGACA
GCAGAAGAGGTCGACTTATCAAAGGATCTCCCTCACTGGAACAAGCTTAAAGCAGATGAGAAGTACTTCA
TCTCTCACATCTTAGCCTTTTTTGCAGCCAGTGATGGAATTGTAAATGAAAATTTGGTGGAGCGCTTTAG
TCAGGAGGTGCAGGTTCCAGAGGCTCGCTGTTTCTATGGCTTTCAAATTCTCATCGAGAATGTTCACTCA
GAGATGTACAGTTTGCTGATAGACACTTACATCAGAGATCCCAAGAAAAGGGAATTTTTATTTAATGCAA
TTGAAACCATGCCCTATGTTAAGAAAAAAGCAGATTGGGCCTTGCGATGGATAGCAGATAGAAAATCTAC
TTTTGGGGAAAGAGTGGTGGCCTTTGCTGCTGTAGAAGGAGTTTTCTTCTCAGGATCTTTTGCTGCTATA
TTCTGGCTAAAGAAGAGAGGTCTTATGCCAGGACTCACTTTTTCCAATGAACTCATCAGCAGAGATGAAG
GACTTCACTGTGACTTTGCTTGCCTGATGTTCCAATACTTAGTAAATAAGCCTTCAGAAGAAAGGGTCAG
GGAGATCATTGTTGATGCTGTCAAAATTGAGCAGGAGTTTTTAACAGAAGCCTTGCCAGTTGGCCTCATT
GGAATGAATTGCATTTTGATGAAACAGTACATTGAGTTTGTAGCTGACAGATTACTTGTGGAACTTGGAT
TCTCAAAGGTTTTTCAGGCAGAAAATCCTTTTGATTTTATGGAAAACATTTCTTTAGAAGGAAAAACAAA
TTTCTTTGAGAAACGAGTTTCAGAGTATCAGCGTTTTGCAGTTATGGCAGAAACCACAGATAACGTCTTC
ACCTTGGATGCAGATTTTTAAAAAACCTCTCGTTTTAAAACTCTATAAACTTGTCATTGGTAAATAGTAG
TCTATTTTCCTCTGCTTAAAAAAAATTTTAAGTATATCCTTTAAAGGACTGGGGGTTTGCTCAAAAGGAA
ATCCAAAACCTATTCTAAACAATTTGCATTTATATAATTTTCCTGTTTAACAACAAGAGTGTGACCTAAA
TGCTTTTGTCTTGTCACTGAAATAAAAGATGGCATTATGTGGTTAAGAGCATGGGGCGAGGGGTCAGACA
TGAGTCTAAGGTTCTGCCCTTACTCCAGTGTGTGACCCTTGGCAAGTCAGTTAATCTTGGTAAACCTCGG
TGTACTTATCTTTAAAATGGGAGTAATAGTAGGTCCTAAATTCATAGAGTGGATATTAGGATTAGGATGC
AAAAATAAATGCTTAACCAACACTACTACTGTTAGCACCACTACTAATTATCATTCATTGATAATATTAA
TTGCAATGATGTTGTAATAAAATACTCTCATTTCCTTAAAATAATTGTGATTCTAGGTCCTAGGATCTAG
AATTAGATCTTTGTATTTTTAATGCTTAGGGGAAGAATATAAGTATCTCCTTAAAAAGAACATAATTCTC
ATTCACGCAAGAATAAGTTCTTTGAATTCCTTAGTATGTAGTGAAGAAAATTTAGTTGTTAGTTGCTTTG
GGAAGCCTACTTATGGAGTGGAAACCAGGAGGTTATCATGGTAGTTGACCTTATAAGAAAAATGATTCTT
CTTCAGAAATTAAAAACATAACTATTGCCAGATTTAGCTCTGGAATGTTTAGAATCAGGCTAGAATAGCA
TTTTCCAAAGAATATTCTAAGAGCTATTAGCTCCTCTAGATATTTTTTTGGGGGAAAAAGGGGATTCTGT
GGTCAGATGAGTTTGGGAAATGCTGAACACTTCATTCTTCTTTAGCAAGTACAGTCAGTACATCAAAGAC
TGAGCAGTTCAGTGGTACATAAATTTATCTCGCCCTGCATATTCCCAACATACTTAACACAGATGTTTTT
TACCTGTTAACATCTCACCCAGCTAGTGTTCCTCAGAACAAAGATTGGAAAAAGCTGGCCGAGAACCATT
TATACATAGAGGAAGGGCTTATGGACTGAGAAAGGGAGAACATGGTAGGGATTATTGAATCATTTCAAAT
TTATACCAGCCTGAATAGTGTACCAGCAATTGACTTAGGCTGTGTTTCTTTATGGTTTTAAAACTCTTGA
GCTGTTATAAGAGATAGTTCTTTTAATGTGACTATGCAACATGATAGCCAATGGTGAGGGAAAAGGAGGT
TTCTCTAGAAGAGTCTGATGAAAGGCCGGGAACCAAGGTTTTTGAGAAGTCTGCCCCTATTTATTTTTAG
TAAGTATCAAGAGGTAGCCTGAGCCTAGTTAGAGTTAGACCTGTCTTTGGATGAAGAAGTCTTAATACTG
AAATACTGAATTTTTAATACATTATTATTTGGTATTCTGTATACCCCTTCAAGCAGTTGTTTCCCATTCC
CAACAAACTGTACTTTATACAATTCTGGATGCTAAAACTTAGAGATTTTCTCTTTGCATAAATTTTGGCT
CCATTCTTTCCATAACAATCTAATCAAAACTGGGAGTTCTCAAGTGAATGCAAAAGGAGCAGGCCATAAC
TTTATTTGTTAGATACACTGTCAGAAACTTGAGATCTTTTGGCCTATGATAATACCATTAATTTTTGCAT
TGCTTCAGTTTGCCAAGTGTTTTTACATCATCTCATTTGATCTCAAAACAGCTTGACAGAGCAACTGTTA
TTGAAATATTACAGATGGAAAGAATGAGGCTCAGGGAAGTTAAATGACTTGGCCAAGATCTGCTCATCGT
CACTGTCTGTACAGTATTTTTTTTTAGAGGTTGTAATGTCTCAGATTTAGTCCTTTACCATCTATGTTGA
TTTGCTTTTGTCTATTTCCTCATTAATTGAATATACTTTAAATATATATATTAAAGTATCAAAATATAGA
GAGACATTTGAACTGTATTCAGGTAATATGTTTAAAGATATTTATATATTGCCATACAAAAACTTAACAT
TTAAAACTGATAATATCTGTAATGACATCAGAATGAAAGAAAAAAAATTGTACAGTGTATATTCCTTTGT
TTTGAATCCAAATCTTTTTCATAGGTAATGACAGATGCCTTAATGTGAAGCTTATTTATAATAGCAATAA
ACCTAACTGGATTTGGATGAAGAAGTCTTAATACTGACATACTGGATTTTTAATGCACTGGTTTGTTATT
TGGTATTCTATCTCTTTTTCCAGGCCTCCAGGTTGCACATTTATTTATTATGTTCAATACTTTGGTTCTT
AGTTCTTAAAGAATCAAGAAGTTGTGTAATCTTTTAAAAATATTATCTTGCAGATAAAGAAAAAAATTAA
GAGTGTGTTTACAACTGTTTTCTCTTTTTTACAGTACATGTATTTAAATCATTGCTATAATAAAGTTAAG
TTCATTAGGAATATAAAAACTTGCAGTTCTATGATAGATTGCATTTATTAAAAATGTTTCATTGTATCAC
ATAGAAATATGGCCAGGAAGGACTTGAGAAGACAGTTTGATCCATTGCTTTTAGACAGGACTGGGTTTTG
CTGTCCAATTATATACAATAATAGTTTTTCTTACAACTAAGCTGGCCCCAGCCTTGTCTTGATATTAATA
CATGAAATTTTTATAATTGTCTCATTGTCTCATTTAGAAACATCCATATTTTTCTGCTTTTTCTATTGCC
ATTTTTTATTTGTGCATGAATTGATTATTGAGAAAATGTAGCAGTTTGCATATTTAAAAATTAATCATTT
TGCATTTTACATTTAAATATGCTAACATCACTGTCATAGAATTCCCAAATTTCATTTGTAGATACTGAAC
TAAGGGCTAATGTCAGGAGCTGATTTTTAATGATAAAGCTGCAGATGGGCTAAATAAAAGCCAAATTAAT
CCTACAATCAGGTATTATGTTTTTAAACCAAGTTGAGTGAATTGGTAGTGGACTTGGGAAATCTTCCCCA
GCAGAATCTGGATGAATGGCACAGAATTGAAATCTCTTTGTTTCCCACCATTTCCCTTTAAGTGCTCTGC
TCCTTTGTAAAAAGTTAAAGATTTGAAAGAGAATCTCATATTCCCGAGGCATTAGGAAGAAAGGATTTAA
TCCCTTCAATTTGGGGCTTAATCTTGTTTAAAAAAATGTAAGTGAAGATGGAAGGCTGGAGAGAATGATT
GCTTTTTGTACAGTTAAATAAGGTCACAATATTCTTACATACTTTGTTTTACAACTGTGTTTTCATTTTT
TCAAATGTCTGGCCATTTAGCAAAGTTATTTACTATTTACTGTGTACATAGAAAGCTTTATTATGTGTGG
TGTATCTAAATTTTTTTTGCTGAAATACATTATGGTCAATCAAGCCAAGCCTGCATGTACAGAATTTGTT
TTTTTTTCAAATAAATTAGTTGTTTTCTTATTTTTTTGGCTTAGTATGTTGAAATAAACTATGGTATCTT
CATCATTTTGTACATTTCCTTTTTGAGGAAGGTTTCTTTATAAGTGCAAGGGCTACCCTAATAAAGGAAT
GTATATACTTACT

As used herein, the term “TP” refers to a gene encoding thymidine phosphorylase, or the corresponding protein product. The terms “TP” and “thymidine phosphorylase” include wild-type forms of the TP gene or protein, as well as variants (e.g., splice variants, truncations, concatemers, and fusion constructs, among others) of wild-type TP proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type TP protein (e.g., SEQ ID NO: 7), provided that the TP variant retains the function of a wild-type TP. Additionally, the terms “TP” and “thymidine phosphorylase” may refer to fusion proteins, or nucleic acids encoding the same, in which the TP is operably linked to another polypeptide, half-life-modifying agent, or therapeutic agent. The term “TP” may refer to the protein or the gene encoding this protein, depending upon the context, as will be appreciated by one of skill in the art.

Exemplary TP amino acid sequence (SEQ ID NO: 7):
MAALMTPGTGAPPAPGDFSGEGSQGLPDPSPEPKQLPELIRMKRDGGRLS
EADIRGFVAAVVNGSAQGAQIGAMLMAIRLRGMDLEETSVLTQALAQSGQ
QLEWPEAWRQQLVDKHSTGGVGDKVSLVLAPALAACGCKVPMISGRGLGH
TGGTLDKLESIPGFNVIQSPEQMQVLLDQAGCCIVGQSEQLVPADGILYA
ARDVTATVDSLPLITASILSKKLVEGLSALVVDVKFGGAAVFPNQEQARE
LAKTLVGVGASLGLRVAAALTAMDKPLGRCVGHALEVEEALLCMDGAGPP
DLRDLVTTLGGALLWLSGHAGTQAQGAARVAAALDDGSALGRFERMLAAQ
GVDPGLARALCSGSPAERRQLLPRAREQEELLAPADGTVELVRALPLALV
LHELGAGRSRAGEPLRLGVGAELLVDVGQRLRRGTPWLRVHRDGPALSGP
QSRALQEALVLSDRAPFAAPSPFAELVLPPQQ
Exemplary TP nucleic acid sequence (SEQ ID NO: 8):
CGACTGCCGAGCTCCGCCCTCCAGGCGGCCCCACCCGCCTGCCGTCCTGG
GGCGCCGCCGCCCCGCCGCCGGCAGTGGACCGCTGTGCGCGAACCCTGAA
CCCTACGGTCCCGACCCGCGGGCGAGGCCGGGTACCTGGGCTGGGATCCG
GAGCAAGCGGGCGAGGGCAGCGCCCTAAGCAGGCATCCCCGCAGGCCCGG
AGCGATGGCAGCCTTGATGACCCCGGGAACCGGGGCCCCACCCGCGCCTG
GTGACTTCTCCGGGGAAGGGAGCCAGGGACTTCCCGACCCTTCGCCAGAG
CCCAAGCAGCTCCCGGAGCTGATCCGCATGAAGCGAGACGGAGGCCGCCT
GAGCGAAGCGGACATCAGGGGCTTCGTGGCCGCTGTGGTGAATGGGAGCG
CGCAGGGCGCACAGATCGGGGCCATGCTGATGGCCATCCGACTTCGGGGC
ATGGATCTGGAGGAGACCTCGGTGCTGACCCAGGCCCTGGCTCAGTCGGG
ACAGCAGCTGGAGTGGCCAGAGGCCTGGCGCCAGCAGCTTGTGGACAAGC
ATTCCACAGGGGGTGTGGGTGACAAGGTCAGCCTGGTCCTCGCACCTGCC
CTGGCGGCATGTGGCTGCAAGGTGCCAATGATCAGCGGACGTGGTCTGGG
GCACACAGGAGGCACCTTGGATAAGCTGGAGTCTATTCCTGGATTCAATG
TCATCCAGAGCCCAGAGCAGATGCAAGTGCTGCTGGACCAGGCGGGCTGC
TGTATCGTGGGTCAGAGTGAGCAGCTGGTTCCTGCGGACGGAATCCTATA
TGCAGCCAGAGATGTGACAGCCACCGTGGACAGCCTGCCACTCATCACAG
CCTCCATTCTCAGTAAGAAACTCGTGGAGGGGCTGTCCGCTCTGGTGGTG
GACGTTAAGTTCGGAGGGGCCGCCGTCTTCCCCAACCAGGAGCAGGCCCG
GGAGCTGGCAAAGACGCTGGTTGGCGTGGGAGCCAGCCTAGGGCTTCGGG
TCGCGGCAGCGCTGACCGCCATGGACAAGCCCCTGGGTCGCTGCGTGGGC
CACGCCCTGGAGGTGGAGGAGGCGCTGCTCTGCATGGACGGCGCAGGCCC
GCCAGACTTAAGGGACCTGGTCACCACGCTCGGGGGCGCCCTGCTCTGGC
TCAGCGGACACGCGGGGACTCAGGCCCAGGGCGCTGCCCGGGTGGCCGCG
GCGCTGGACGACGGCTCGGCCCTTGGCCGCTTCGAGCGGATGCTGGCGGC
GCAGGGCGTGGATCCCGGTCTGGCCCGAGCCCTGTGCTCGGGAAGTCCCG
CAGAACGCCGGCAGCTGCTGCCTCGCGCCCGGGAGCAGGAGGAGCTGCTG
GCGCCCGCAGATGGCACCGTGGAGCTGGTCCGGGCGCTGCCGCTGGCGCT
GGTGCTGCACGAGCTCGGGGCCGGGCGCAGCCGCGCTGGGGAGCCGCTCC
GCCTGGGGGTGGGCGCAGAGCTGCTGGTCGACGTGGGTCAGAGGCTGCGC
CGTGGGACCCCCTGGCTCCGCGTGCACCGGGACGGCCCCGCGCTCAGCGG
CCCGCAGAGCCGCGCCCTGCAGGAGGCGCTCGTACTCTCCGACCGCGCGC
CATTCGCCGCCCCCTCGCCCTTCGCAGAGCTCGTTCTGCCGCCGCAGCAA
TAAAGCTCCTTTGCCGCGAAAAAAAAAAA

As used herein, the term “SUCLA2” refers to a gene encoding succinate-CoA ligase ADP-forming beta subunit, or the corresponding protein product. The terms “SUCLA2” and “succinate-CoA ligase ADP-forming beta subunit” include wild-type forms of the SUCLA2 gene or protein, as well as variants (e.g., splice variants, truncations, concatemers, and fusion constructs, among others) of wild-type SUCLA2 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SUCLA2 protein (e.g., SEQ ID NO: 9), provided that the SUCLA2 variant retains the function of a wild-type SUCLA2. Additionally, the terms “SUCLA2” and “succinate-CoA ligase ADP-forming beta subunit” may refer to fusion proteins, or nucleic acids encoding the same, in which the SUCLA2 is operably linked to another polypeptide, half-life-modifying agent, or therapeutic agent. The term “SUCLA2” may refer to the protein or the gene encoding this protein, depending upon the context, as will be appreciated by one of skill in the art.

Exemplary SUCLA2 amino acid sequence (SEQ ID NO:
9):
MAASMFYGRLVAVATLRNHRPRTAQRAAAQVLGSSGLFNNHGLQVQQQQQ
RNLSLHEYMSMELLQEAGVSVPKGYVAKSPDEAYAIAKKLGSKDVVIKAQ
VLAGGRGKGTFESGLKGGVKIVFSPEEAKAVSSQMIGKKLFTKQTGEKGR
ICNQVLVCERKYPRREYYFAITMERSFQGPVLIGSSHGGVNIEDVAAESP
EAIIKEPIDIEEGIKKEQALQLAQKMGFPPNIVESAAENMVKLYSLFLKY
DATMIEINPMVEDSDGAVLCMDAKINFDSNSAYRQKKIFDLQDWTQEDER
DKDAAKANLNYIGLDGNIGCLVNGAGLAMATMDIIKLHGGTPANFLDVGG
GATVHQVTEAFKLITSDKKVLAILVNIFGGIMRCDVIAQGIVMAVKDLEI
KIPVVVRLQGTRVDDAKALIADSGLKILACDDLDEAARMVVKLSEIVTLA
KQAHVDVKFQLPI
Exemplary SUCLA2 nucleic acid sequence (SEQ ID NO:
10):
CCGCCTGTCGCCTGTGCGCCTGCGCGCGGCGCCGAGGGGACGGGGTCCGA
CTCAGAAATGGCGGCCTCCATGTTCTACGGCAGGCTAGTGGCCGTGGCCA
CCCTTCGGAACCACCGGCCTCGGACGGCCCAGCGGGCTGCTGCTCAGGTT
CTGGGAAGTTCTGGATTGTTTAATAACCATGGACTCCAAGTACAGCAGCA
ACAGCAAAGGAATCTCTCACTACATGAATACATGAGTATGGAATTATTGC
AAGAAGCTGGTGTCTCCGTTCCCAAAGGATATGTGGCAAAGTCACCAGAT
GAAGCTTATGCAATTGCCAAAAAATTAGGTTCAAAAGATGTCGTGATAAA
GGCACAGGTTTTAGCTGGTGGTAGAGGAAAAGGAACATTTGAAAGTGGCC
TCAAAGGAGGAGTGAAGATAGTTTTCTCTCCAGAAGAAGCAAAAGCTGTT
TCTTCACAAATGATTGGGAAAAAATTGTTTACCAAGCAAACGGGAGAAAA
GGGCAGAATATGCAATCAAGTATTGGTCTGTGAGCGAAAATATCCCAGGA
GAGAATACTACTTTGCAATAACAATGGAAAGGTCATTTCAAGGTCCTGTA
TTAATAGGAAGTTCACATGGTGGTGTCAACATTGAAGATGTTGCTGCTGA
GTCTCCTGAAGCAATAATTAAAGAACCTATTGATATTGAAGAAGGCATCA
AAAAGGAACAAGCTCTCCAGCTTGCACAGAAGATGGGATTTCCACCTAAT
ATTGTGGAATCAGCAGCAGAAAACATGGTCAAGCTTTACAGCCTTTTTCT
GAAATACGATGCAACCATGATAGAAATAAATCCAATGGTGGAAGATTCAG
ATGGAGCTGTATTGTGTATGGATGCAAAGATCAATTTTGACTCTAATTCA
GCCTATCGCCAAAAGAAAATCTTTGATCTACAGGACTGGACCCAGGAAGA
TGAAAGGGACAAAGATGCTGCTAAGGCAAATCTCAACTACATTGGCCTCG
ATGGAAATATAGGCTGCCTAGTAAATGGTGCTGGTTTGGCTATGGCCACA
ATGGATATAATAAAACTTCATGGAGGGACTCCAGCCAACTTCCTTGATGT
TGGTGGTGGTGCTACAGTCCATCAAGTAACAGAAGCATTTAAGCTTATCA
CTTCAGATAAAAAGGTACTGGCTATTCTGGTCAACATTTTTGGAGGAATC
ATGCGCTGTGATGTTATTGCACAGGGTATAGTCATGGCAGTAAAAGACTT
GGAAATTAAAATACCTGTTGTGGTACGGTTACAAGGTACACGAGTCGATG
ATGCTAAGGCACTGATAGCGGACAGTGGACTTAAAATACTTGCTTGTGAT
GACTTGGATGAAGCTGCTAGAATGGTTGTAAAGCTCTCTGAAATAGTGAC
CTTAGCGAAGCAAGCACATGTGGATGTGAAATTTCAGTTGCCAATATGAT
CTGAAAACCCAGTGGATGGCTGAAGGTGTTAAATGTGCTATAATCATTAA
GAATACTGTGTTCTGTGTTATTGTTCTTTTTCTTTTTAGTGTGTGGAGAT
TGTAATTGCCATCTAGGCACACAAACATTTAAAAGGATTTGGACTGCATT
TAATTGTACCATTCAGAATGGACTGTTTGTACGAAGCATGTATAATGCAG
TTATCTTCTTTCTTTTGTCGCAGCCAGTCTTTTTTGCTTCTCCTACAAAA
CGTAACTTGCAATTTGCCAGTTTATTATTGTTGGATACAAAGTTCTTCAT
TGATAAGAGTCCTATAAATAAGATAAATACGAAGATAAAGCTTTATTCTT
TAGTGTTAAAATACAGTATATCTAATAACTAGCCTCATTAGTAGAGCAGT
ATATTAAAACAATGTTTTATGTAAAAAGTGTTTATCTTCAGCACCAAATA
CATGATAAATGTATCAATCACTATTTATAAACAGAGCTTTCAAACACTCC
TCAGAATATTCTTCTAAGTATTTTGATGAAGTAACTTTGTAATTATTTGA
ACATTGTTTTAATCATTAGGAAACACTGATTAACTGCAAGTCTTCATGAT
TCTGTCATATTAAGAAACACCTGTAGGTTTGCTTCAAATAAAGGCATATA
TACCAAGGACTTACAGACAAAATTAAGAATGTCAATTTAAGTTAATAAAA
ATCTCCCAATATGAAAAAAAAAAAAAAAAAAA

As used herein, the term “SUCLG1” refers to a gene encoding succinate-CoA ligase ADP-forming alpha subunit, or the corresponding protein product. The terms “SUCLG1” and “succinate-CoA ligase ADP-forming alpha subunit” include wild-type forms of the SUCLG1 gene or protein, as well as variants (e.g., splice variants, truncations, concatemers, and fusion constructs, among others) of wild-type SUCLG1 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type SUCLG1 protein (e.g., SEQ ID NO: 11), provided that the SUCLG1 variant retains the function of a wild-type SUCLG1. Additionally, the terms “SUCLG1” and “succinate-CoA ligase ADP-forming alpha subunit” may refer to fusion proteins, or nucleic acids encoding the same, in which the SUCLG1 is operably linked to another polypeptide, half-life-modifying agent, or therapeutic agent. The term “SUCLG1” may refer to the protein or the gene encoding this protein, depending upon the context, as will be appreciated by one of skill in the art.

Exemplary SUCLG1 amino acid sequence (SEQ ID NO:
11):
MTATLAAAADIATMVSGSSGLAAARLLSRSFLLPQNGIRHCSYTASRQHL
YVDKNTKIICQGFTGKQGTFHSQQALEYGTKLVGGTTPGKGGQTHLGLPV
FNTVKEAKEQTGATASVIYVPPPFAAAAINEAIEAEIPLVVCITEGIPQQ
DMVRVKHKLLRQEKTRLIGPNCPGVINPGECKIGIMPGHIHKKGRIGIVS
RSGTLTYEAVHQTTQVGLGQSLCVGIGGDPFNGTDFIDCLEIFLNDSATE
GIILIGEIGGNAEENAAEFLKQHNSGPNSKPVVSFIAGLTAPPGRRMGHA
GAIIAGGKGGAKEKISALQSAGVVVSMSPAQLGTTIYKEFEKRKML
Exemplary SUCLG1 nucleic acid sequence (SEQ ID NO:
12):
GTCATTGGCGTATGACCGCAACCCTTGCCGCTGCCGCTGACATCGCTACC
ATGGTCTCCGGCAGCAGCGGCCTCGCCGCCGCCCGTCTCCTGTCGCGCAG
CTTCCTCCTGCCGCAGAATGGAATTCGGCATTGTTCCTACACAGCTTCTC
GGCAACATCTCTATGTTGATAAAAATACAAAGATTATTTGCCAGGGTTTC
ACTGGCAAACAGGGCACCTTTCACAGCCAGCAGGCATTGGAATATGGCAC
CAAACTCGTTGGAGGAACCACTCCAGGGAAAGGAGGCCAGACACATCTGG
GCTTACCTGTCTTTAATACTGTGAAGGAGGCCAAAGAACAGACAGGAGCA
ACGGCTTCTGTCATTTATGTTCCTCCGCCTTTTGCTGCTGCTGCCATTAA
TGAAGCTATTGAGGCAGAAATTCCCTTGGTTGTGTGTATCACTGAAGGAA
TTCCCCAGCAGGACATGGTACGAGTCAAGCACAAACTGCTGCGCCAGGAA
AAGACAAGGCTAATTGGGCCCAACTGCCCTGGAGTCATCAATCCTGGAGA
ATGTAAAATTGGCATCATGCCTGGCCATATTCACAAAAAAGGAAGGATTG
GCATTGTGTCCAGATCTGGCACCCTGACTTATGAAGCAGTTCACCAAACA
ACGCAAGTTGGATTGGGGCAGTCTTTGTGCGTTGGCATTGGAGGTGATCC
TTTTAATGGAACAGATTTTATTGACTGCCTCGAAATCTTTTTGAACGATT
CTGCCACAGAAGGCATCATATTGATTGGTGAAATTGGTGGTAATGCAGAA
GAGAATGCTGCAGAATTTTTGAAGCAACATAATTCAGGTCCAAATTCCAA
GCCTGTAGTGTCCTTCATTGCTGGTTTAACTGCTCCTCCTGGGAGAAGAA
TGGGTCATGCCGGGGCAATTATTGCTGGAGGAAAAGGTGGAGCTAAAGAG
AAGATCTCTGCCCTTCAGAGTGCAGGAGTTGTGGTCAGTATGTCTCCTGC
ACAGCTGGGAACCACGATCTACAAGGAATTTGAAAAGAGGAAGATGCTAT
GAAAGAAAAAAAAAATTCCTAAAACTGTGGAATGGATCACGTAGACATGT
AACCCAGCAGCAGTTTGCTTCTGTTGTCCACTGATTAATCAGCCTATGTG
CCTGACACTGGTCTTGCAGTACAACTGGAAGCCAAAACAAGGTGGAAGAT
GTCCTGAATTAAGATGTTTTCACCACATTGTATTACAGAGACAGCCAATA
AATCTACTATTTGATTTCAA

As used herein, the term “MPV17” refers to a gene encoding mitochondrial inner membrane protein MPV17, or the corresponding protein product. The terms “MPV17” and “mitochondrial inner membrane protein MPV17” include wild-type forms of the MPV17 gene or protein, as well as variants (e.g., splice variants, truncations, concatemers, and fusion constructs, among others) of wild-type MPV17 proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type MPV17 protein (e.g., SEQ ID NO: 13), provided that the MPV17 variant retains the function of a wild-type MPV17. Additionally, the terms “MPV17” and “mitochondrial inner membrane protein MPV17” may refer to fusion proteins, or nucleic acids encoding the same, in which the MPV17 is operably linked to another polypeptide, half-life-modifying agent, or therapeutic agent. The term “MPV17” may refer to the protein or the gene encoding this protein, depending upon the context, as will be appreciated by one of skill in the art.

Exemplary MPV17 amino acid sequence (SEQ ID NO:
13):
MALWRAYQRALAAHPWKVQVLTAGSLMGLGDIISQQLVERRGLQEHQRGR
TLTMVSLGCGFVGPVVGGWYKVLDRFIPGTTKVDALKKMLLDQGGFAPCF
LGCFLPLVGALNGLSAQDNWAKLQRDYPDALITNYYLWPAVQLANFYLVP
LHYRLAVVQCVAVIWNSYLSWKAHRL
Exemplary MPV17 nucleic acid sequence (SEQ ID NO:
14):
AGTTCCTAGGCCAGCCTGTCACGTGGGAGGGAGGCTCGGCGCTCAGGAAG
CATGGCACTCTGGCGGGCATACCAGCGGGCCCTGGCCGCTCACCCGTGGA
AAGTACAGGTCCTGACAGCTGGGTCCCTGATGGGCCTGGGTGACATTATC
TCACAGCAGCTGGTGGAGAGGCGGGGTCTGCAGGAACACCAGAGAGGCCG
GACTCTGACCATGGTGTCCCTGGGCTGTGGCTTTGTGGGCCCTGTGGTAG
GAGGCTGGTACAAGGTTTTGGATCGGTTCATCCCTGGCACCACCAAAGTG
GATGCACTGAAGAAGATGTTGTTGGATCAGGGGGGCTTTGCCCCGTGTTT
TCTAGGCTGCTTTCTCCCACTGGTAGGGGCACTTAATGGACTGTCAGCCC
AGGACAACTGGGCCAAACTACAGCGGGATTATCCTGATGCCCTTATCACC
AACTACTATCTATGGCCTGCTGTGCAGTTAGCCAACTTCTACCTGGTCCC
CCTTCATTACAGGTTGGCCGTTGTCCAATGTGTTGCTGTTATCTGGAACT
CCTACCTGTCCTGGAAGGCACATCGGCTCTAAGCCTGCCTCACTCCATCG
TTTCCACCTTGCAGTGATGCAGCTTGACCCTGGAACGGTCAGACAACCTC
CTCAAAGTGGGCATACCAGTTTCCACGGGGTTGGGTTGCCGGTCAGAGCT
TAAGAGGACTAGCACCCTGCAATGCCCCTCTTCACTCTAAAATGTACACT
GACTGCTITAGAGCCCTTGATAATAGTCTTATTCCCACCACATACTAGGC
ACTCCATAAATATCTGTTGAACCTTCATGACCTTATCAACTTTACACCCA
TATCCCAGCAAATGCCACTCATCCCCACTCTTCATAGACACATTTGTTAC
TCTAACCCTGCCTAGGCTTCTTGTAGCTCCAGCTCTTTAGAGACTCCCGG
AACCCTTTATATGGTGCCTCAGTAAATATGTTATTAAATATGTAATCCGG
AA

As used herein, the term “POLG” refers to a gene encoding DNA polymerase gamma, catalytic subunit, or the corresponding protein product. The terms “POLG” and “DNA polymerase gamma, catalytic subunit” include wild-type forms of the POLG gene or protein, as well as variants (e.g., splice variants, truncations, concatemers, and fusion constructs, among others) of wild-type POLG proteins and nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type POLG protein (e.g., SEQ ID NO: 15), provided that the POLG variant retains the function of a wild-type POLG. Additionally, the terms “POLG” and “DNA polymerase gamma, catalytic subunit” may refer to fusion proteins, or nucleic acids encoding the same, in which the POLG is operably linked to another polypeptide, half-life-modifying agent, or therapeutic agent. The term “POLG” may refer to the protein or the gene encoding this protein, depending upon the context, as will be appreciated by one of skill in the art.

Exemplary POLG amino acid sequence (SEQ ID NO: 15):
MSRLLWRKVAGATVGPGPVPAPGRWVSSSVPASDPSDGQRRRQQQQQQQQQQQQQPQQPQVLSSEGGQLR
HNPLDIQMLSRGLHEQIFGQGGEMPGEAAVRRSVEHLQKHGLWGQPAVPLPDVELRLPPLYGDNLDQHFR
LLAQKQSLPYLEAANLLLQAQLPPKPPAWAWAEGWTRYGPEGEAVPVAIPEERALVFDVEVCLAEGTCPT
LAVAISPSAWYSWCSQRLVEERYSWTSQLSPADLIPLEVPTGASSPTQRDWQEQLVVGHNVSFDRAHIRE
QYLIQGSRMRFLDTMSMHMAISGLSSFQRSLWIAAKQGKHKVQPPTKQGQKSQRKARRGPAISSWDWLDI
SSVNSLAEVHRLYVGGPPLEKEPRELFVKGTMKDIRENFQDLMQYCAQDVWATHEVFQQQLPLFLERCPH
PVTLAGMLEMGVSYLPVNQNWERYLAEAQGTYEELQREMKKSLMDLANDACQLLSGERYKEDPWLWDLEW
DLQEFKQKKAKKVKKEPATASKLPIEGAGAPGDPMDQEDLGPCSEEEEFQQDVMARACLQKLKGTTELLP
KRPQHLPGHPGWYRKLCPRLDDPAWTPGPSLLSLQMRVTPKLMALTWDGFPLHYSERHGWGYLVPGRRDN
LAKLPTGTTLESAGVVCPYRAIESLYRKHCLEQGKQQLMPQEAGLAEEFLLTDNSAIWQTVEELDYLEVE
AEAKMENLRAAVPGQPLALTARGGPKDTQPSYHHGNGPYNDVDIPGCWFFKLPHKDGNSCNVGSPFAKDF
LPKMEDGTLQAGPGGASGPRALEINKMISFWRNAHKRISSQMVVWLPRSALPRAVIRHPDYDEEGLYGAI
LPQVVTAGTITRRAVEPTWLTASNARPDRVGSELKAMVQAPPGYTLVGADVDSQELWIAAVLGDAHFAGM
HGCTAFGWMTLQGRKSRGTDLHSKTATTVGISREHAKIFNYGRIYGAGQPFAERLLMQFNHRLTQQEAAE
KAQQMYAATKGLRWYRLSDEGEWLVRELNLPVDRTEGGWISLQDLRKVQRETARKSQWKKWEVVAERAWK
GGTESEMFNKLESIATSDIPRTPVLGCCISRALEPSAVQEEFMTSRVNWVVQSSAVDYLHLMLVAMKWLF
EEFAIDGRFCISIHDEVRYLVREEDRYRAALALQITNLLTRCMFAYKLGLNDLPQSVAFFSAVDIDRCLR
KEVTMDCKTPSNPTGMERRYGIPQGEALDIYQIIELTKGSLEKRSQPGP
Exemplary POLG nucleic acid sequence (SEQ ID NO: 16):
GCGGACCGGCCGGGTGGAGGCCACACGCTACCCCGAGGCTGCGTAGGCCGCGCGAAGGGGGACGCCGTGC
CGTGGGCCTGGGGTCGGGGGAGCAGCAGACCGGGAAGCACCGATTTGGGGTGGAAGGCAGGCATGGTCAA
ACCCATTTCACTGACAGGAGAGCAGAGACAGGACGTGTCTCTCTCCACGTCTTCCAGCCAGTAAAAGAAG
CCAAGCTGGAGCCCAAAGCCAGGTGTTCTGACTCCCAGCGTGGGGGTCCCTGCACCAACCATGAGCCGCC
TGCTCTGGAGGAAGGTGGCCGGCGCCACCGTCGGGCCAGGGCCGGTTCCAGCTCCGGGGCGCTGGGTCTC
CAGCTCCGTCCCCGCGTCCGACCCCAGCGACGGGCAGCGGCGGCGGCAGCAGCAGCAGCAGCAGCAGCAG
CAGCAGCAACAGCAGCCTCAGCAGCCGCAAGTGCTATCCTCGGAGGGCGGGCAGCTGCGGCACAACCCAT
TGGACATCCAGATGCTCTCGAGAGGGCTGCACGAGCAAATCTTCGGGCAAGGAGGGGAGATGCCTGGCGA
GGCCGCGGTGCGCCGCAGCGTCGAGCACCTGCAGAAGCACGGGCTCTGGGGGCAGCCAGCCGTGCCCTTG
CCCGACGTGGAGCTGCGCCTGCCGCCCCTCTACGGGGACAACCTGGACCAGCACTTCCGCCTCCTGGCCC
AGAAGCAGAGCCTGCCCTACCTGGAGGCGGCCAACTTGCTGTTGCAGGCCCAGCTGCCCCCGAAGCCCCC
GGCTTGGGCCTGGGCGGAGGGCTGGACCCGGTACGGCCCCGAGGGGGAGGCCGTACCCGTGGCCATCCCC
GAGGAGCGGGCCCTGGTGTTCGACGTGGAGGTCTGCTTGGCAGAGGGAACTTGCCCCACATTGGCGGTGG
CCATATCCCCCTCGGCCTGGTATTCCTGGTGCAGCCAGCGGCTGGTGGAAGAGCGTTACTCTTGGACCAG
CCAGCTGTCGCCGGCTGACCTCATCCCCCTGGAGGTCCCTACTGGTGCCAGCAGCCCCACCCAGAGAGAC
TGGCAGGAGCAGTTAGTGGTGGGGCACAATGTTTCCTTTGACCGAGCTCATATCAGGGAGCAGTACCTGA
TCCAGGGTTCCCGCATGCGTTTCCTGGACACCATGAGCATGCACATGGCCATCTCAGGGCTAAGCAGCTT
CCAGCGCAGTCTGTGGATAGCAGCCAAGCAGGGCAAACACAAGGTCCAGCCCCCCACAAAGCAAGGCCAG
AAGTCCCAGAGGAAAGCCAGAAGAGGCCCAGCGATCTCATCCTGGGACTGGCTGGACATCAGCAGTGTCA
ACAGTCTGGCAGAGGTGCACAGACTTTATGTAGGGGGGCCTCCCTTAGAGAAGGAGCCTCGAGAACTGTT
TGTGAAGGGCACCATGAAGGACATTCGTGAGAACTTCCAGGACCTGATGCAGTACTGTGCCCAGGACGTG
TGGGCCACCCATGAGGTTTTCCAGCAGCAGCTACCGCTCTTCTTGGAGAGGTGTCCCCACCCAGTGACTC
TGGCCGGCATGCTGGAGATGGGTGTCTCCTACCTGCCTGTCAACCAGAACTGGGAGCGTTACCTGGCAGA
GGCACAGGGCACTTATGAGGAGCTCCAGCGGGAGATGAAGAAGTCGTTGATGGATCTGGCCAATGATGCC
TGCCAGCTGCTCTCAGGAGAGAGGTACAAAGAAGACCCCTGGCTCTGGGACCTGGAGTGGGACCTGCAAG
AATTTAAGCAGAAGAAAGCTAAGAAGGTGAAGAAGGAACCAGCCACAGCCAGCAAGTTGCCCATCGAGGG
GGCTGGGGCCCCTGGTGATCCCATGGATCAGGAAGACCTCGGCCCCTGCAGTGAGGAGGAGGAGTTTCAA
CAAGATGTCATGGCCCGCGCCTGCTTGCAGAAGCTGAAGGGGACCACAGAGCTCCTGCCCAAGCGGCCCC
AGCACCTTCCTGGACACCCTGGATGGTACCGGAAGCTCTGCCCCCGGCTAGACGACCCTGCATGGACCCC
GGGCCCCAGCCTCCTCAGCCTGCAGATGCGGGTCACACCTAAACTCATGGCACTTACCTGGGATGGCTTC
CCTCTGCACTACTCAGAGCGTCATGGCTGGGGCTACTTGGTGCCTGGGCGGCGGGACAACCTGGCCAAGC
TGCCGACAGGTACCACCCTGGAGTCAGCTGGGGTGGTCTGCCCCTACAGAGCCATCGAGTCCCTGTACAG
GAAGCACTGTCTCGAACAGGGGAAGCAGCAGCTGATGCCCCAGGAGGCCGGCCTGGCGGAGGAGTTCCTG
CTCACTGACAATAGTGCCATATGGCAAACGGTAGAAGAACTGGATTACTTAGAAGTGGAGGCTGAGGCCA
AGATGGAGAACTTGCGAGCTGCAGTGCCAGGTCAACCCCTAGCTCTGACTGCCCGTGGTGGCCCCAAGGA
CACCCAGCCCAGCTATCACCATGGCAATGGACCTTACAACGACGTGGACATCCCTGGCTGCTGGTTTTTC
AAGCTGCCTCACAAGGATGGTAATAGCTGTAATGTGGGAAGCCCCTTTGCCAAGGACTTCCTGCCCAAGA
TGGAGGATGGCACCCTGCAGGCTGGCCCAGGAGGTGCCAGTGGGCCCCGTGCTCTGGAAATCAACAAAAT
GATTTCTTTCTGGAGGAACGCCCATAAACGTATCAGCTCCCAGATGGTGGTGTGGCTGCCCAGGTCAGCT
CTGCCCCGTGCTGTGATCAGGCACCCCGACTATGATGAGGAAGGCCTCTATGGGGCCATCCTGCCCCAAG
TGGTGACTGCCGGCACCATCACTCGCCGGGCTGTGGAGCCCACATGGCTCACCGCCAGCAATGCCCGGCC
TGACCGAGTAGGCAGTGAGTTGAAAGCCATGGTGCAGGCCCCACCTGGCTACACCCTTGTGGGTGCTGAT
GTGGACTCCCAAGAGCTGTGGATTGCAGCTGTGCTTGGAGACGCCCACTTTGCCGGCATGCATGGCTGCA
CAGCCTTTGGGTGGATGACACTGCAGGGCAGGAAGAGCAGGGGCACTGATCTACACAGTAAGACAGCCAC
TACTGTGGGCATCAGCCGTGAGCATGCCAAAATCTTCAACTACGGCCGCATCTATGGTGCTGGGCAGCCC
TTTGCTGAGCGCTTACTAATGCAGTTTAACCACCGGCTCACACAGCAGGAGGCAGCTGAGAAGGCCCAGC
AGATGTACGCTGCCACCAAGGGCCTCCGCTGGTATCGGCTGTCGGATGAGGGCGAGTGGCTGGTGAGGGA
GTTGAACCTCCCAGTGGACAGGACTGAGGGTGGCTGGATTTCCCTGCAGGATCTGCGCAAGGTCCAGAGA
GAAACTGCAAGGAAGTCACAGTGGAAGAAGTGGGAGGTGGTTGCTGAACGGGCATGGAAGGGGGGCACAG
AGTCAGAAATGTTCAATAAGCTTGAGAGCATTGCTACGTCTGACATACCACGTACCCCGGTGCTGGGCTG
CTGCATCAGCCGAGCCCTGGAGCCCTCGGCTGTCCAGGAAGAGTTTATGACCAGCCGTGTGAATTGGGTG
GTACAGAGCTCTGCTGTTGACTACTTACACCTCATGCTTGTGGCCATGAAGTGGCTGTTTGAAGAGTTTG
CCATAGATGGGCGCTTCTGCATCAGCATCCATGACGAGGTTCGCTACCTGGTGCGGGAGGAGGACCGCTA
CCGCGCTGCCCTGGCCTTGCAGATCACCAACCTCTTGACCAGGTGCATGTTTGCCTACAAGCTGGGTCTG
AATGACTTGCCCCAGTCAGTCGCCTTTTTCAGTGCAGTCGATATTGACCGGTGCCTCAGGAAGGAAGTGA
CCATGGATTGTAAAACCCCTTCCAACCCAACTGGGATGGAAAGGAGATACGGGATTCCCCAGGGTGAAGC
GCTGGATATTTACCAGATAATTGAACTCACCAAAGGCTCCTTGGAAAAACGAAGCCAGCCTGGACCATAG
CACTGCCTGGAGGCTCTGTATTTGCTCCCGTGGAGCTTCATCGGGGTGGTGCAGGCTCCCAAACTCAGGC
TTTCAGCTGTGCTTTTTGCAAAAGGGCTTGCCTAAGGCCAGCCATTTTTCAGTAGCAGGACCTGCCAAGA
AGATTCCTTCTAACTGAAGGTGCAGTTGAATTCAGTGGGTTCAGAACCAAGATGCCAACATCGGTGTGGA
CTACAGGACAAGGGGCATTGTTGCTTGTTGGGTAAAAATGAAGCAGAAGCCCCAAAGTTCACATTAACTC
AGGCATTTCATTTATTTTTTCCTTTTCTTCTTGGCTGGTTCTTTGTTCTGTCCCCCATGCTCTGATGCAG
TGCCCTAGAAGGGGAAAGAATTAATGCTCTAACGTGATAAACCTGCTCCAAGGCAGTGGAAATAAAAAGA
AGGAAAAAAAAGACTCTATCTTCTCAAAAAAAAAAAAAAAAA

Standard methods in molecular biology are described Sambrook, Fritsch and Maniatis Molecular Cloning, A Laboratory Manual, Cold Spring Harbor Laboratory Press, Cold Spring Harbor, NY (1982 & 1989 2nd Edition, 2001 3rd Edition); Sambrook and Russell Molecular 35 Cloning, 3rd ed., Cold Spring Harbor Laboratory Press, Cold Spring Harbor, NY (2001); Wu Recombinant DNA, Vol. 217, Academic Press, San Diego, CA) (1993). Standard methods also appear in Ausbel, et al. Current Protocols in Molecular Biology, Vols. 1-4, John Wiley and Sons, Inc. New York, NY (2001).

### Mitochondrial DNA Depletion Syndrome Treatment and Prevention

The compositions and methods of the present disclosure may be used to treat, prevent, and/or cure a variety of pathologies. For example, gene therapy methods described herein may involve administration of one or more compositions containing a nucleic acid encoding a functional protein so as to treat, prevent, and/or cure a disease associated with mitochondrial dysfunction. For the treatment of a mitochondrial DNA depletion syndrome, such as a deficiency of TK2, dGK, TP, p53R2, SUCLA2, SUCLG1, MPV17, and/or POLG, the gene therapy may involve administration of one or more compositions containing a gene encoding a functional form of the deficient protein. The treatment can also involve the administration of pharmacological therapy in conjunction with the gene therapy. Such a treatment protocol involving gene therapy alone or in combination with pharmacological therapy may be used to treat a variety of disorders characterized by unbalanced nucleoside pools, such as those found in mitochondrial DNA depletion syndrome.

Mitochondrial DNA (mtDNA) depletion syndrome (MDS) comprises several severe autosomal diseases characterized by a reduction in mtDNA copy number in affected tissues. Most of the MDS causative nuclear genes encode proteins that belong to the mtDNA replication machinery or are involved in deoxyribonucleoside triphosphate (dNTP) metabolism making all of these diseases candidates for gene therapy.

One form of MDS is thymidine kinase deficiency or TK2. TK2 encoded by the nuclear gene, TK2, is a mitochondrial matrix protein that phosphorylates thymidine and cytidine nucleosides to generate deoxythymidine monophosphate (TMP) and deoxycytidine monophosphate (dCMP), which in turn, are converted to deoxynucleotide triphosphates (dNTPs) required for mitochondrial DNA synthesis. Autosomal recessive TK2 mutations cause devastating neuromuscular weakness with severe depletion of mitochondrial DNA (mtDNA) in infants and children, as well as progressive external ophthalmoplegia with mtDNA multiple deletions in adults. Many patients cannot walk and require some type of mechanical ventilation and feeding tube. The central nervous system is variably involved in these disorders, with symptoms that include seizures, encephalopathy, cognitive impairment, and hearing loss. Less than 7% of patients live more than 42 years.

Based on clinical and molecular genetics findings of patients thus diagnosed, three disease presentations were identified: i) infantile-onset (<1 year-old) myopathy with onset of weakness in the first year of life with severe mtDNA depletion and early mortality; ii) childhood-onset (>1-11 years-old) myopathy with severe mtDNA depletion; and iii) late-onset myopathy (>12 years-old) with mild weakness at onset and slow progression to loss of ambulation, respiratory insufficiency, or both, often with chronic progressive external ophthalmoparesis in adolescence or adulthood in association with mtDNA multiple deletions, reduced mtDNA copy number, or both. See Garone, et al. 2018.

Attempts to study the pathogenesis and test therapies for TK2 deficiency using cultured fibroblasts from patients have been unsuccessful, because the replicating cells failed to manifest mtDNA depletion. In contrast, a homozygous Tk2 H126N knock-in mutant (Tk2^{-/-}) mouse model, manifests a phenotype that is strikingly similar to the human infantile encephalomyopathy caused by TK2 mutations, characterized by onset at age 10 days with decreased ambulation, unstable gait, coarse tremor, growth retardation, and depletion of mitochondrial DNA (mtDNA) progressing rapidly to early death at age 14 to 16 days, which is a time period analogous to the human infantile-onset disease (Akman, et al. 2008; Dorado, et al. 2011). This mouse model has been used to show the administration of oral dC/dT prolonged delayed the onset of clinical symptoms of TK2 deficiency and prolonged the lives of the mice by two- to three-fold ('092 application). It was also used to show the administration of oral dCMP/TMP delayed clinical symptoms and prolonged life ('207 application).

Exemplary Findings from AAV Vectors Encoding TK2 in Mouse Models of MDS

As shown herein using a mouse model of Tk2 deficiency, the disease can be cured by replacing the mutant gene with a normal gene that encodes a functional Tk2 protein. An exemplary gene therapy described herein involves the use of an AAV (e.g., an AAV2 or AAV9 virus) containing a transgene encoding TK2. It has been discovered that administration of an AAV9 virus expressing human TK2 (referred to as “AAV9-hTK2” in the Examples provided below) prolonged the lifespan of TK2 mutant mice up to a maximum lifespan of about 4 months and an average of about 3 months. This was more than double the lifespan of the oral dC/dT pharmacological-treated TK2 mutant mice, which was an average of 43 days, and five times longer than the untreated TK2 mutant mice which was 18 days (see, e.g., Example 2). Weight also increased from about 55% over mice with dC/dT treatment alone and about 80% over the weight of wild type mice (see, e.g., Example 3).

Muscle strength (normalized to weight) and motor function assessed by Rotarod test was performed at postnatal day 60 and showed no differences between the treated TK2 mutant mice and untreated wild-type mice (see, e.g., Examples 3 and 4). Wild-type mice showed no adverse side-effects related to the therapy and were overall healthy.

Activity and levels of OXPHOS enzyme complexes in brain of treated mutant mice were similar to that showed in untreated and treated wild-type mice (see, e.g., Examples 7 and 8).

Additionally, treated mutant mice never showed head tremor, characteristic of untreated and dC+dT treated Tk2^{-/-} mice, confirming that rescue of mitochondrial function in central nervous system prevents pathology.

Mitochondrial DNA was also rescued by the AAV9 administration in most tissue of the mutant mice (see, e.g., Example 6).

Treatment with 4.2×10¹¹ vc of AAV9-hTK2 at P1 does not efficiently transduce kidney cells, as previously observed in other studies. Consequently, TK2 activity in treated mutant mice was very poor compared to wild-type mice and there were signs of mtDNA depletion as soon as post-natal day 29. Histology and biochemical analysis showed compromised kidney function, which may be the underlying condition leading to early death of Tk2^{-/-} mice (see, e.g., Example 8).

However, when the TK2 mutant mice were administered with a combination of the AAV9-hTK2 and an AAV2 virus expressing human TK2 gene (AAV2-hTK2), they survived significantly longer despite having lower levels of total viral dose. The mutant mice treated with both constructs grew as much as the mice treated with only AAV9-hTK2 and had slightly higher levels of mtDNA than mice treated with only AAV9-hTK2. They had equal growth and strength and motor coordination as the mutant mice treated with AAV9-hTK2 only as well as a decrease of protein in their urine and lower levels of index BUN (see, e.g., Example 9).

Additionally, when the mutant mice were treated with both AAV9-hTK2 and AAV2-hTK2 were further administered oral dC+dT in water, they survived even longer and have increased growth and mtDNA levels in the liver and kidney and decreased protein in their urine (see, e.g., Example 10).

Additionally, the TK2 mutant mice are treated with the oral dC/dT as described in the '092 application or the oral dCMP/TMP as described in '207 application, starting at an early age, e.g., postnatal day 4, and subsequently administered the AAV9-hTK2 vector, alone or in combination with the AAV2-hTK2 vector, at a later age, ranging from one month to about two months, with about 21 days being ideal. The AAV2-hTK2 vector can be administered simultaneously with the AAV9-hTK2 vector or subsequently. These mutant mice survived longer than the mutant mice administered only the AAV9-hTK2 and have increased growth and strength, and mtDNA and RCEs are restored in all tissues including kidney (see, e.g., Examples 11 and 12).

Methods of Treating, Preventing, and/or Curing Mitochondrial Diseases in Patients

Patients who would benefit from the administration of the described gene therapy include those diagnosed with a deficiency in one or more of TK2, dGK, TP, p53R2, SUCLA2, SUCLG1, MPV17, and/or POLG. In these patients, compositions containing a nucleic acid encoding one or more of the deficient proteins (e.g., viral vectors, such as AAV vectors, containing such nucleic acids) may be administered to the patient. These compositions may be administered alone or in combination with pharmacological agents, such as deoxynucleosides (e.g., dC and dT or mixtures thereof), or deoxyribonucleoside monophosphates (e.g., dCMP and TMP or mixtures thereof). For example, the subject may have a mitochondrial DNA depletion syndrome and the treatment comprises administering (i) a composition containing a viral vector (e.g. AAV, such as an AAV that targets at least muscle tissue, optionally at least muscle tissue and CNS, for example AAV9) containing a nucleic acid encoding one or more of the deficient proteins, and (ii) a composition comprising deoxynucleosides (e.g., dC and dT or mixtures thereof), or deoxyribonucleoside monophosphates (e.g., dCMP and TMP or mixtures thereof). In one embodiment, the mitochondrial DNA depletion syndrome is TK2 deficiency and the viral vector (e.g. AAV, such as an AAV that targets at least muscle tissue, optionally at least muscle tissue and CNS, for example AAV9) comprises a nucleic acid encoding TK2. In a particular embodiment, the mitochondrial DNA depletion syndrome is TK2 deficiency and the viral vector is AAV9 comprising a nucleic acid encoding TK2.

In some embodiments, the present disclosure provides methods of treating, preventing, curing, and/or reducing the severity or extent of TK2 deficiency by administering to a subject in need thereof a therapeutically effective amount of a composition, such as a viral vector (e.g., an AAV), comprising a nucleic acid encoding TK2. In some embodiments, the viral vector is an AAV, such as AAV9 or AAV2. In some embodiments, the composition (e.g., viral vector, such as an AAV) comprising a nucleic acid encoding TK2 is administered as soon as TK2 deficiency is diagnosed or suspected. In some embodiments, the amount of AAV comprising the transgene administered is about 4.2×10¹¹ or 4.2×10¹⁰ genome or vector or vector copies.

The disclosure also provides methods of treating, preventing, curing, and/or reducing the severity or extent of TK2 deficiency by administering to a subject in need thereof a therapeutically effective amount of a first composition (e.g., viral vector, such as AAV) containing a nucleic acid encoding TK2 and further comprising administering to the subject a therapeutically effective amount of a second composition (e.g., viral vector, such as AAV) containing a nucleic acid encoding TK2. In some embodiments, the first and second AAV are each independently an AAV2 or AAV9 vector encoding TK2. In some embodiments, the first AAV vector is administered prior to the second AAV vector. In some embodiments, the first composition (e.g., AAV vector) is administered as soon as TK2 deficiency is diagnosed or suspected, and the second composition (e.g., AAV vector) is administered at a time point after the first composition. In some embodiments, the second composition (e.g., AAV vector) is administered within days of the first composition (e.g., AAV vector). In some embodiments, the second composition (e.g., AAV vector) is administered within weeks of the first composition (e.g., AAV vector). In some embodiments, the second composition (e.g., AAV vector) is administered months after the first composition (e.g., AAV vector). In some embodiments, the first composition (e.g., AAV vector) and the second composition (e.g., AAV vector) are administered simultaneously at any given time point. In some embodiments, the two compositions (e.g., AAV vectors) are present within the same larger composition, and in some embodiments, the two are separate compositions.

In some embodiments, the method further includes further administering a therapeutically effective amount of a pharmacological agent, such as dC and/or dT. The pharmacological agent may be administered at the same time that the first composition (e.g., viral vector, such as an AAV vector) is administered or at a time point after the first composition is administered. In some embodiments, the pharmacological agent, such as dC and/or dT, is administered within days of the first composition. In some embodiments, the pharmacological agent, such as dC and/or dT, is administered within weeks of the first composition, and in some embodiments, the pharmacological agent, such as dC and/or dT, is administered months after the first composition. In some embodiments, the first composition, second composition, and the pharmacological agent are administered simultaneously at any given time point.

In some embodiments, the first composition (e.g., viral vector, such as an AAV, encoding a transgene described herein, for example TK2) is administered as soon as TK2 deficiency is known or suspected. The pharmacological agent (e.g., dC and/or dT) may be administered starting from about 7 days to about 35 days after the administration of the first composition, starting from about 14 days to about 28 days after the administration of the first composition, starting from about 14 days to about 21 days after the administration of the first composition, or starting from about 21 days after the administration of the first composition. The second composition (e.g., viral vector, such as an AAV, encoding a transgene described herein, for example TK2) may be administered starting from about 14 days to about 45 days after the administration of the first composition, starting from about 21 days to about 35 days after the administration of the first composition, starting from about 21 days to about 30 days after the administration of the first composition, or starting from about 29 days after the administration of the first composition. In some embodiments, administration of the pharmacological agent (e.g., dC and/or dT) continues throughout the subject's lifetime.

In some embodiments, the disclosure provides methods of treating, preventing, curing, and/or reducing the severity or extent of TK2 deficiency by administering to a subject in need thereof a therapeutically effective amount of a pharmacological agent (e.g., dC and/or dT) and further includes administering to the subject a therapeutically effective amount of a composition containing a viral vector, such as an AAV vector (e.g. rAAV), containing a nucleic acid encoding TK2. For example, the composition may contain an AAV2 or AAV9 containing a nucleic acid encoding TK2. In certain embodiments, the AAV is AAV9. In some embodiments, the pharmacological agent is administered prior to the AAV vector. In some embodiments, the pharmacological agent is administered as soon as TK2 deficiency is diagnosed or suspected, and the AAV vector is administered at a time point after. In some embodiments, the AAV vector is administered within days of the pharmacological agent, and in some embodiments, the AAV vector is administered within weeks of the pharmacological agent. In some embodiments, the AAV vector is administered months after the pharmacological agent. In some embodiments, the pharmacological agent and the AAV vector are administered simultaneously at any given time point. In some embodiments, a second AAV vector (e.g., AAV2 or AAV9) encoding TK2 is also administered. The first and second AAV vector may be the same or different. For example, the first AAV vector may be an AAV2 vector (i.e., containing AAV2 ITRs and AAV2 capsid proteins) and the second AAV vector may be an AAV9 vector (e.g., containing AAV9 ITRs and AAV9 capsid proteins). In some embodiments, one or both of the first and second vectors is a pseudotyped AAV vector containing AAV2 ITRs and capsid proteins from a different AAV serotype (e.g., an AAV2/1, AAV2/8, or AAV2/9 vector). In some embodiments, the first AAV vector is administered simultaneously or later in time relative to administration of the second AAV vector. The first and second vectors may be administered in the same composition or in different compositions.

In some embodiments, the disclosure provides methods of treating, preventing and/or reducing the severity or extent of TK2 deficiency by administering to a subject in need thereof a therapeutically effective amount of a pharmacological agent containing dCMP and/or TMP and additionally administering to the subject a therapeutically effective amount of a composition containing an AAV vector (e.g., an AAV2 or AAV9) containing a nucleic acid encoding TK2. In some embodiments, the dCMP and/or TMP is administered prior to administration of the AAV vector. In some embodiments, the dCMP and/or TMP is administered as soon as TK2 deficiency is diagnosed or suspected, and the AAV vector is administered at a time point after. In some embodiments, the AAV vector is administered within days of dCMP and/or TMP administration, and in some embodiments, the AAV vector is administered within weeks of the dCMP and/or TMP. In some embodiments, the AAV vector is administered months after the dCMP and/or TMP. In some embodiments, the dCMP and/or TMP and the AAV vector are administered simultaneously at any time point. In some embodiments, an additional AAV vector (e.g., an AAV2 or AAV9 vector) containing a nucleic acid encoding TK2 is also administered to the subject. The second AAV vector can be administered simultaneously or later in time relative to administration of the first AAV vector. The first and second AAV vectors may be administered as part of the same composition or as different composition.

All of the aforementioned methods can be used to restore function of a dysfunctional TK2 protein in a subject with TK2 deficiency.

A parallel defect of dGK, due to autosomal recessive mutations in DGUOK with deficiencies in dGMP and dAMP, causes mtDNA depletion typically manifesting as early childhood-onset hepatocerebral disease (Mandel, et al. 2001). These patients would benefit from the administration of an AAV vector comprising the nucleic acid that encodes dGK and at least one deoxypurine, dG or dA, or mixtures thereof. Similarly, patients having deficiencies of TP, p53R2, SUCLA2, SUCLG1, MPV17, and/or POLG may be treated using the compositions and methods of the disclosure, for example, by providing such a patient with a therapeutically effective amount of a composition (e.g., a viral vector, such as an AAV vector) containing a transgene encoding the deficient protein. The composition may be provided to the patient alone or in combination with a pharmacological agent, such as a deoxynucleoside or deoxynucleoside monophosphate described herein.

For example, other forms of MDS, as well as other disorders related to unbalanced nucleotide pools, can be treated by administration of compositions (e.g., viral vectors, such as AAV vectors) containing nucleic acids encoding one or more of TK2, dGK, TP, p53R2, SUCLA2, SUCLG1, MPV17, and/or POLG. The composition may be administered alone or in combination with a pharmacological agent, such as a deoxynucleosides (e.g., dA, dG, dC, or dT, or mixture thereof) or deoxynucleoside monophosphate (e.g., dAMP, dGMP, dCMP, or TMP or mixture thereof). These disorders include, without limitation, deficiencies related to RRM2B (encoding p53R2, the p53 inducible small subunit of ribonucleotide reductase, RNR) and mutations in TYMP (encoding thymidine phosphorylase, TP) which cause mitochondrial neurogastrointestinal encephalomyopathy (MNGIE). Additional nuclear genes that disrupt mitochondrial dNTP pools include but are not limited to SUCLA2, SUCLG1 and MPV17.

### Assessing the Condition of a Patient Having a Mitochondrial Disease

Patients that exhibit the phenotype discussed above for TK2 deficiency including the most typical presentation of progressive muscle disease characterized by generalized hypotonia, proximal muscle weakness, loss of previously acquired motor skills, poor feeding, and respiratory difficulties, can be tested to definitively diagnose the disease.

If the clinical presentation is highly suspicious for mtDNA depletion syndrome, molecular genetic testing using a panel of genes known to cause mtDNA depletion syndrome should be performed (Chanprasert, et al. 2012). The TK2 gene is the only gene in which mutations are known to cause TK2-related mitochondrial DNA depletion syndrome. This testing can include a sequence analysis of the entire coding and exon/intron junction regions of TK2 for sequence variants and deletion/duplication. If compound heterozygous or homozygous deleterious mutations are identified in the sequence analysis, the diagnosis of TK2 deficiency is confirmed, and thus, the subject would benefit from the therapy described herein. If sequence analysis does not identify two compound heterozygous or homozygous deleterious mutations, deletion/duplication analysis should be considered to determine and/or confirm a TK2 deficiency diagnosis.

Further tests to determine and/or confirm a TK2 deficiency diagnosis may include testing serum creatine kinase (CK) concentration, electromyography, histopathology on skeletal muscle, mitochondrial DNA (mtDNA) content (copy number), and electron transport chain (ETC) activity in skeletal muscle. If one or more of the following is found in these tests, the TK2 deficiency is determined and/or confirmed. Elevated CK concentration as compared to healthy controls can indicate TK2 deficiency. A skeletal muscle biopsy can be performed, and then a mtDNA content analysis in skeletal muscle performed. If the skeletal muscle biopsy shows prominent variance in fiber size, variable sarcoplasmic vacuoles, variable increased connective tissue, and ragged red fibers as well as increased succinate dehydrogenase (SDH) activity and low to absent cytochrome c oxidase (COX) activity, and mtDNA copy number is severely reduced (typically less than 20% of age- and tissue-matched healthy controls), a diagnosis of TK2 deficiency can be determined and/or confirmed (Chanprasert, et al. 2012).

Additionally, TK2 deficiency is inherited in an autosomal recessive manner. Thus, a sibling of an affected patient can be tested as early as possible after birth to diagnose the disease.

In all of these examples, gene therapy alone or in combination with deoxynucleoside therapy or deoxyribonucleoside monophosphate therapy should be started as soon as possible after a diagnosis of TK2 deficiency.

### Recombinant AAV Vectors

“Recombinant AAV (rAAV) vectors” described herein generally include a transgene (e.g., encoding TK2, dGK, TP, p53R2, SUCLA2, SUCLG1, MPV17, and/or POLG). The transgene is flanked by 5′ and 3′ ITRs, and may be operably linked to one or more regulatory elements in a manner that permits transgene transcription, translation, and/or expression in a cell of a target tissue. Such regulatory elements may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). The AAV vector may then be delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product of interest (e.g., TK2). Components of exemplary AAV vectors that may be used in conjunction with the compositions and methods of the disclosure are described below.

Any AAV serotype or combination of AAV serotype can be used in the methods and compositions of the present invention. Because the methods and compositions of the present invention are for the treatment and cure of mitochondrial disorders, AAV serotypes that target at least muscle, or at least muscle and the central nervous system can be used in some embodiments and include but are not limited to AAV1, AAV2, AAV4, AAV5, AAV6, AAV7, AAV8, and AAV9.

In some embodiments, AAV9 serotype, which has a wide tropism, is used.

### Components of AAV Vectors

The AAV vectors described herein may contain cis-acting 5′ and 3′ ITRs (See, e.g., Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, (1989) and Fisher et al., (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA).

For nucleic acids encoding proteins, a polyadenylation sequence generally is inserted following the transgene sequences and before the 3′ AAV ITR sequence. An rAAV construct useful in the present invention may also contain an intron, desirably located between the promoter/enhancer sequence and the transgene. One possible intron sequence is derived from SV-40, and is referred to as the SV-40 T intron sequence.

Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available (see, e.g., Sambrook et al, and references cited). Such a motif may be useful, for example, for instances in which multiple genes or portions thereof are expressed from the same AAV vector.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a 13-actin promoter, a phosphoglycerol kinase (PGK) promoter, and an EF1a promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system (WO 98/10088); a ecdysone insect promoter (No et al., Proc. Natl. Acad. Sci. USA 93:3346-3351 (1996)), a tetracycline-repressible system (Gossen et al., Proc. Natl. Acad. Sci. USA 89:5547-5551 (1992)), a tetracycline-inducible system (Gossen et al., Science 268:1766-1769 (1995), a RU486-inducible system (Wang et al., Nat. Biotech. 15:239-243 (1997) and Wang et al., Gene 5 Ther. 4:432-441 (1997)) and a rapamycin-inducible system (Magari et al., J Clin. Invest. 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, a native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner.

In some embodiments, one or more bindings sites for one or more of miRNAs are incorporated in a transgene of a rAAV vector, to inhibit the expression of the transgene in one or more tissues of a subject harboring the transgenes. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

For example, a 3′UTR site which would inhibit the expression of the transgene in the liver can be incorporated into a transgene. This would be beneficial for transgenes which encode therapeutic proteins which are toxic to the liver as most of the virus administered (approximately 60 to 90%) is eventually found in the liver. Thus suppressing the therapeutic gene expression in liver relieves the burden from liver cells.

In some embodiments, the AAV vector will be modified to be a self-complementing AAV. A self-complementing AAV carries complementary sequence of the transgene (i.e., a double copy of the transgene). Self complementation makes the gene more stable after it enters the cell.

### Transgene Coding Sequences

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In embodiments of the current invention the transgenes would encode a functional protein including but not limited to TK2, dGK, p53R2, TP, SUCLA2, SUCLG1, POLG1, and a mitochondrial membrane protein MPV17.

The human TK2 gene (Gene ID: 7084) can be used to obtain a transgene encoding a functional TK2 protein. The human DGUOK (Gene ID: 1716) can be used to obtain a transgene encoding a functional dGK protein. The human TYMP gene (Gene ID: 1890) can be used to obtain a transgene encoding a functional TP protein. The human RRM2B gene (Gene ID: 50584) can be used to obtain a transgene encoding a functional p53R2 protein. The human SUCLA2 gene (Gene ID: 8803) can be used to obtain a transgene encoding a functional SUCLA2 protein. The human SUCLG1 gene (Gene ID: 8802) can be used to obtain a transgene encoding a functional SUCLG1 protein. The human POLG gene (Gene ID: 5428) can be used to obtain a transgene encoding a functional POLG1 protein. The human MPV17 gene (Gene ID: 4538) can be used to obtain a transgene encoding a functional mitochondrial membrane protein MPV17.

### Codon Optimization of Transgene Coding Sequences

Codon optimization of the transgene coding sequences can increase the efficiency of the gene therapy. Thus, in some embodiments, a nucleic acid that is at least 70% identical to the coding sequence of the transgene encoding the therapeutic protein (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence) is used.

Codon optimization tools are known in the art.

Exemplary codon optimized nucleic acids are as follows.

Exemplary codon optimized TK2 nucleic acid (SEQ ID NO: 17):
ATGGGAGCATTTTGCCAGCGACCTAGTTCCGATAAAGAACAGGAGAAAGAGAAGAAGAGCGTCATCTG
TGTGGAAGGTAACATCGCTAGTGGCAAAACGACCTGTTTGGAATTTTTCTCTAATGCCACCGATGTGG
AGGTGCTTACAGAGCCTGTTAGTAAATGGCGAAATGTTCGAGGACATAATCCGCTGGGACTGATGTAT
CACGATGCCTCCAGATGGGGACTGACCCTTCAGACTTATGTCCAACTTACAATGCTTGACAGGCACAC
TAGACCGCAGGTTAGCAGCGTGAGGCTGATGGAGCGCTCAATTCACTCTGCCAGGTATATATTCGTTG
AGAACCTCTACCGATCTGGTAAGATGCCTGAGGTAGATTACGTAGTGCTGAGTGAATGGTTTGACTGG
ATTCTTCGCAACATGGATGTATCAGTGGATCTCATTGTTTACCTTAGGACTAACCCGGAGACGTGTTA
TCAGCGCTTGAAAAAGCGCTGCCGGGAAGAGGAAAAAGTGATTCCTCTGGAATACTTGGAAGCTATTC
ATCACCTTCACGAAGAGTGGCTGATAAAAGGTTCCCTGTTTCCCATGGCGGCCCCCGTGCTTGTCATA
GAAGCGGATCACCACATGGAAAGGATGCTCGAACTTTTCGAGCAAAATCGAGACCGGATTCTCACGCC
GGAGAATCGAAAACATTGCCCATAG
Exemplary codon optimized dGK nucleic acid (SEQ ID NO: 18):
ATGGCTGCAGGGAGGCTTTTTTTGTCTAGATTGAGGGCACCTTTTAGCTCCATGGCGAAGTCTCCGCT
TGAGGGGGTGAGTAGCAGCCGGGGGCTCCATGCTGGCAGAGGGCCCCGGAGGCTGTCTATTGAGGGGA
ATATTGCAGTTGGTAAGAGCACGTTTGTTAAATTGCTCACGAAGACCTATCCAGAATGGCACGTTGCT
ACAGAACCAGTAGCTACATGGCAAAACATTCAAGCAGCAGGCACACAGAAGGCTTGCACTGCACAGTC
TCTCGGAAACCTGCTCGATATGATGTACAGAGAGCCCGCCCGGTGGTCATACACGTTTCAGACCTTCA
GCTTTCTTTCTCGGTTGAAGGTACAGTTGGAGCCCTTCCCAGAGAAGTTGCTTCAAGCACGGAAACCT
GTACAGATATTCGAACGATTGCATTTCGAGGCGCTGATGAACATACCTGTGCTTGTTCTGGATGTGAA
CGACGATTTTTCCGAGGAAGTTACTAAGCAAGAGGACCTCATGCGGGAGGTAAACACGTTCGTTAAGA
ACCTGTAA
Exemplary codon optimized TP nucleic acid (SEQ ID NO: 19):
ATGGCAGCATTGATGACACCAGGAACGGGGGCTCCGCCTGCGCCTGGCGATTTTTCAGGAGAGGGGAG
TCAAGGTCTGCCGGACCCTTCACCGGAACCCAAACAGCTTCCCGAGCTTATTAGAATGAAGCGAGATG
GGGGTCGCCTCTCTGAGGCTGACATACGCGGCTTTGTTGCGGCGGTCGTCAACGGCTCAGCACAAGGT
GCGCAAATAGGAGCGATGCTGATGGCTATCAGGCTGAGGGGAATGGATTTGGAAGAGACCAGTGTGCT
GACTCAAGCGCTGGCACAGAGCGGCCAGCAGCTTGAATGGCCAGAAGCTTGGCGCCAACAATTGGTCG
ATAAACACTCAACCGGGGGCGTCGGGGACAAAGTCTCCCTTGTACTGGCACCTGCTCTGGCTGCGTGC
GGATGTAAGGTTCCTATGATTAGCGGGCGGGGCTTGGGACATACGGGAGGAACGTTGGACAAACTCGA
ATCCATCCCTGGTTTCAACGTGATTCAGAGCCCTGAACAAATGCAAGTACTCCTGGATCAGGCAGGCT
GCTGTATTGTTGGCCAAAGCGAACAACTCGTGCCTGCGGATGGTATCCTCTATGCTGCCAGGGATGTG
ACCGCCACAGTTGACTCCCTCCCGCTGATAACAGCGTCAATTTTGAGTAAGAAGCTCGTCGAAGGGCT
CAGCGCTCTTGTGGTTGATGTAAAATTTGGGGGGGCCGCAGTATTCCCCAACCAGGAACAAGCACGGG
AACTGGCAAAAACCCTGGTTGGTGTGGGCGCTTCACTGGGCCTGAGAGTTGCCGCTGCCTTGACCGCC
ATGGATAAGCCGCTCGGTAGGTGCGTAGGTCATGCACTTGAAGTAGAAGAAGCCCTTTTGTGCATGGA
TGGGGCCGGTCCCCCTGACTTGCGGGATCTCGTAACCACGCTTGGCGGCGCGTTGCTTTGGCTTTCTG
GACACGCTGGTACCCAAGCGCAAGGGGCAGCAAGAGTCGCAGCAGCGCTTGATGACGGATCAGCTCTT
GGGCGATTTGAAAGAATGCTTGCAGCTCAGGGCGTAGATCCAGGGCTGGCGCGGGCCCTTTGCTCAGG
TTCTCCAGCAGAGCGCCGACAGCTCCTTCCCAGGGCGCGAGAGCAGGAAGAATTGCTGGCCCCGGCTG
ATGGTACCGTCGAACTCGTACGGGCTTTGCCGCTGGCTCTTGTTTTGCACGAACTGGGGGCTGGGCGG
AGTCGCGCCGGTGAACCTCTCAGACTCGGTGTGGGTGCGGAGCTCCTCGTTGACGTCGGGCAACGACT
TCGCAGGGGAACCCCTTGGCTTAGGGTACACAGGGACGGGCCAGCACTCAGCGGCCCTCAGTCCAGGG
CCCTTCAAGAAGCTCTCGTGCTGAGTGATCGAGCGCCTTTTGCCGCTCCCTCACCATTTGCTGAATTG
GTATTGCCACCCCAGCAGTAA
Exemplary codon optimized p53R2 nucleic acid (SEQ ID NO: 20):
ATGCTGCTTCTCAGACTGCCACCCCACCGCAGTCATGCTTCCCCACTTGACTGTAAACTTCA
AGATAGGTGCCGGAAGTGTTACTCACCCCGATCAGGACAAGCGTGCCCTCCTGCGTTGGCGG
CCGCCTGGCTTCGGCGGTGTGAACGCCGAGGAGGTCGGCCGAGGGGTGGGCGACGCAAGGAA
CTGACCCTTGGCCTGCGCCCTGCACGGTGTTCAGCTCCCGGTCCTGCCAAGGATGATGCATG
GAGACCTCAGGCAGGTCGCTCCTCCTCAGATACCAACGAGAGTGAAATTAAATCTAATGAAG
AACCCCTTCTTCGCAAGAGTAGTCGCAGGTTCGTGATATTCCCCATACAATATCCTGATATC
TGGAAAATGTACAAGCAAGCCCAAGCATCATTCTGGACCGCAGAAGAGGTTGATCTCAGTAA
GGACCTCCCACACTGGAACAAACTGAAAGCCGACGAAAAATATTTCATATCCCATATACTTG
CTTTCTTTGCAGCCTCAGATGGAATTGTGAATGAAAACTTGGTAGAGCGCTTCTCTCAAGAG
GTCCAAGTTCCTGAAGCACGATGCTTTTACGGATTTCAAATCCTGATAGAAAATGTACATTC
CGAAATGTATTCCTTGCTTATAGACACGTATATCAGAGATCCCAAAAAAAGGGAGTTCTTGT
TCAATGCGATCGAGACCATGCCATATGTTAAGAAAAAAGCCGATTGGGCCCTGAGGTGGATA
GCTGATAGGAAGTCTACATTTGGGGAACGCGTTGTTGCCTTCGCCGCCGTCGAGGGAGTCTT
CTTCTCCGGATCCTTCGCTGCTATCTTTTGGCTGAAAAAACGCGGCTTGATGCCTGGTTTGA
CCTTTAGCAATGAGTTGATATCACGGGATGAAGGGCTGCATTGTGACTTTGCCTGCCTTATG
TTCCAGTACTTGGTGAACAAACCGTCTGAGGAACGGGTAAGGGAAATCATAGTGGATGCAGT
AAAAATAGAGCAGGAGTTCCTTACTGAAGCGCTCCCCGTAGGGCTCATTGGCATGAACTGCA
TTCTTATGAAGCAGTACATCGAGTTCGTGGCCGATAGACTTTTGGTGGAGCTCGGGTTCTCA
AAGGTTTTTCAGGCTGAAAATCCTTTTGATTTTATGGAGAACATATCTCTGGAGGGCAAGAC
TAACTTTTTTGAAAAAAGAGTATCCGAGTATCAAAGGTTTGCCGTGATGGCAGAAACAACAG
ATAATGTCTTCACACTTGATGCGGATTTTTAA
Exemplary codon optimized SUCLA2 nucleic acid (SEQ ID NO: 21):
ATGGCGGCTTCTATGTTCTATGGACGCCTGGTCGCCGTTGCCACGTTGCGGAATCACAGGCCCCGAAC
TGCACAAAGGGCTGCCGCTCAGGTGCTTGGAAGCAGCGGATTGTTCAACAATCACGGCCTCCAAGTTC
AGCAACAGCAGCAGAGAAACCTCTCTCTGCATGAGTATATGAGTATGGAACTGCTCCAGGAAGCAGGG
GTGTCTGTACCAAAGGGCTACGTAGCAAAAAGCCCCGACGAGGCGTACGCCATAGCTAAAAAACTGGG
AAGTAAGGACGTAGTTATCAAAGCACAGGTTCTGGCCGGTGGCCGCGGTAAGGGAACCTTCGAGAGTG
GTTTGAAAGGCGGTGTCAAGATCGTATTTAGTCCCGAGGAGGCCAAGGCTGTAAGTTCCCAGATGATC
GGCAAAAAATTGTTCACAAAGCAAACCGGCGAAAAGGGTAGAATTTGCAACCAAGTACTTGTCTGCGA
AAGAAAGTATCCGAGAAGGGAGTATTATTTCGCTATAACCATGGAAAGATCATTCCAAGGTCCCGTGC
TCATCGGCAGTTCACACGGGGGCGTTAATATAGAGGATGTCGCTGCAGAGTCTCCGGAGGCCATAATT
AAGGAGCCTATAGACATCGAGGAAGGCATTAAAAAAGAACAAGCCTTGCAACTGGCACAAAAAATGGG
TTTTCCTCCGAACATCGTCGAGAGTGCCGCAGAAAATATGGTGAAACTGTACAGCCTGTTTTTGAAGT
ACGATGCGACAATGATAGAAATTAATCCGATGGTCGAGGACTCAGATGGAGCCGTGCTTTGTATGGAC
GCCAAAATTAACTTCGATTCAAACAGCGCTTATCGACAGAAGAAAATTTTCGATTTGCAAGATTGGAC
CCAAGAGGATGAGCGAGACAAAGATGCCGCCAAAGCAAATCTCAATTATATAGGACTCGATGGTAATA
TTGGATGTTTGGTCAATGGTGCCGGTCTCGCGATGGCAACTATGGATATAATCAAGTTGCATGGTGGG
ACTCCCGCCAACTTTCTTGACGTCGGGGGAGGCGCCACCGTTCACCAAGTTACGGAGGCTTTCAAACT
CATAACCTCTGACAAGAAGGTGCTGGCGATTCTTGTAAATATCTTTGGTGGCATTATGCGGTGTGATG
TTATTGCCCAGGGAATAGTAATGGCTGTCAAAGATTTGGAGATTAAAATACCCGTCGTGGTGCGGCTC
CAAGGTACTCGAGTAGATGATGCTAAGGCTCTGATTGCCGACAGCGGGCTGAAGATTCTGGCATGTGA
TGATTTGGACGAGGCAGCGAGAATGGTCGTGAAGCTCTCAGAAATAGTAACTTTGGCGAAACAGGCTC
ATGTCGATGTAAAGTTCCAACTTCCTATATGA
Exemplary codon optimized SUCLG1 nucleic acid (SEQ ID NO: 22):
ATGACCGCTACGCTTGCGGCTGCAGCCGACATCGCGACAATGGTCTCTGGAAGTTCAGGCTTGGCCGC
GGCTCGGCTTCTGAGTAGAAGTTTCTTGTTGCCGCAAAACGGTATCAGGCACTGCTCCTACACCGCCA
GTAGACAACACCTTTATGTTGATAAGAACACGAAGATAATTTGCCAAGGATTCACCGGTAAACAGGGG
ACCTTCCATTCCCAGCAGGCACTGGAATATGGAACTAAATTGGTAGGCGGCACTACGCCTGGGAAGGG
TGGCCAAACACATCTTGGTCTTCCCGTTTTTAACACAGTGAAGGAGGCTAAAGAACAAACGGGGGCAA
CGGCTAGCGTTATCTATGTCCCACCCCCGTTTGCCGCTGCAGCCATAAATGAGGCGATTGAAGCCGAG
ATCCCGCTTGTAGTCTGCATAACGGAGGGAATTCCGCAACAAGACATGGTGCGAGTAAAGCACAAGCT
TCTTCGACAGGAAAAAACAAGACTGATAGGTCCGAATTGTCCTGGCGTAATTAACCCCGGTGAATGCA
AAATCGGAATTATGCCGGGACATATTCACAAAAAAGGCCGAATCGGGATAGTCAGCAGATCAGGCACC
TTGACATACGAAGCGGTTCACCAGACTACGCAAGTCGGTTTGGGACAAAGCCTTTGCGTCGGTATCGG
TGGCGACCCATTTAACGGGACGGATTTCATTGACTGTCTGGAGATCTTCCTCAACGATTCCGCGACAG
AAGGAATCATTTTGATAGGAGAAATAGGCGGGAACGCCGAAGAGAATGCAGCGGAATTCCTCAAACAA
CATAACTCTGGCCCTAATAGTAAGCCGGTGGTATCATTCATAGCCGGTCTTACAGCGCCGCCGGGTCG
CAGAATGGGCCACGCAGGTGCAATTATAGCGGGGGGCAAGGGAGGTGCCAAAGAAAAGATATCCGCTC
TTCAAAGCGCAGGTGTAGTAGTTAGTATGAGTCCAGCTCAACTGGGCACAACCATCTACAAAGAATTT
GAAAAGCGGAAAATGCTTTGA
Exemplary codon optimized MPV17 nucleic acid (SEQ ID NO: 23):
ATGGCCCTCTGGAGAGCTTACCAACGAGCCTTGGCCGCGCACCCTTGGAAGGTACAGGTCTTGACCGC
CGGATCTTTGATGGGTCTTGGAGATATTATTTCTCAACAGTTGGTTGAACGGCGAGGCCTCCAGGAAC
ACCAAAGGGGGAGAACCCTTACAATGGTAAGTCTGGGTTGCGGATTCGTCGGGCCTGTCGTGGGGGGA
TGGTATAAGGTTCTGGATCGCTTTATACCGGGGACCACCAAGGTCGATGCCTTGAAGAAGATGTTGCT
GGATCAGGGAGGATTCGCTCCGTGTTTTTTGGGATGTTTTCTGCCACTTGTTGGGGCTCTGAACGGAC
TGTCCGCGCAGGATAACTGGGCGAAGTTGCAACGCGACTACCCAGACGCCCTGATAACAAATTACTAT
CTCTGGCCAGCAGTTCAGTTGGCCAATTTTTACCTGGTACCTCTCCACTATCGCCTTGCTGTAGTACA
GTGTGTCGCCGTCATCTGGAACTCATACCTTTCATGGAAGGCTCATAGATTGTAA
Exemplary codon optimized POLG nucleic acid (SEQ ID NO: 24):
ATGTCCAGGCTGCTCTGGCGGAAGGTCGCAGGCGCCACTGTCGGTCCAGGACCAGTTCCAGCTCCCGG
TCGCTGGGTGAGCAGCAGCGTGCCAGCTAGCGATCCGAGCGACGGTCAGCGGCGACGGCAACAACAGC
AGCAACAGCAACAACAGCAACAACAACAGCCCCAGCAGCCTCAGGTTCTCAGTTCCGAGGGTGGCCAA
CTCCGACACAACCCACTGGATATACAGATGCTCTCCCGCGGTCTCCACGAACAAATATTCGGACAAGG
GGGTGAGATGCCGGGAGAGGCTGCGGTCAGGCGCAGTGTAGAACATCTCCAGAAACACGGGTTGTGGG
GCCAACCGGCCGTTCCTCTCCCCGATGTTGAACTGCGGCTTCCACCTCTCTACGGTGATAATCTGGAC
CAGCACTTTAGACTGCTCGCTCAAAAGCAGAGTCTCCCTTACCTGGAAGCCGCTAACCTCCTGCTCCA
AGCCCAATTGCCCCCTAAACCGCCAGCCTGGGCTTGGGCGGAGGGATGGACGAGGTATGGACCCGAAG
GGGAGGCTGTGCCAGTTGCTATACCAGAGGAACGCGCTCTGGTTTTCGACGTAGAGGTTTGTCTCGCG
GAGGGAACTTGTCCTACACTGGCTGTAGCAATTTCCCCTTCAGCCTGGTACAGCTGGTGCTCTCAGAG
ATTGGTGGAAGAAAGGTATAGCTGGACTAGCCAGCTGAGTCCCGCGGACCTCATTCCACTTGAGGTAC
CCACCGGGGCGTCAAGCCCAACTCAGAGGGACTGGCAGGAACAATTGGTAGTTGGGCATAATGTGAGT
TTTGACAGGGCTCATATCCGCGAACAGTATCTTATCCAGGGCTCTAGAATGCGATTCCTTGACACGAT
GAGCATGCACATGGCAATCAGCGGACTTAGTTCCTTTCAGAGGTCATTGTGGATTGCAGCCAAGCAGG
GAAAGCATAAGGTCCAACCCCCGACAAAACAAGGTCAGAAATCCCAGAGAAAAGCCCGGCGAGGCCCC
GCCATCAGTTCCTGGGATTGGTTGGATATCAGTAGTGTGAATAGCCTTGCTGAGGTGCATCGCCTGTA
TGTGGGTGGACCCCCACTTGAGAAAGAGCCTAGGGAGCTCTTCGTCAAAGGCACCATGAAGGATATTA
GAGAGAATTTTCAAGATCTCATGCAATACTGCGCACAAGACGTATGGGCAACGCATGAGGTCTTTCAA
CAGCAACTCCCCCTCTTTTTGGAACGATGTCCACATCCCGTCACTCTTGCTGGGATGTTGGAAATGGG
TGTAAGTTATTTGCCAGTCAATCAAAATTGGGAGAGATACTTGGCTGAAGCGCAGGGTACATATGAGG
AACTTCAGCGAGAAATGAAAAAAAGTCTTATGGATTTGGCCAATGACGCCTGCCAGCTGCTTTCCGGT
GAGCGGTACAAAGAAGATCCATGGCTTTGGGATCTCGAATGGGATTTGCAGGAATTTAAACAGAAAAA
GGCCAAGAAGGTCAAGAAAGAGCCAGCTACAGCCTCAAAGCTCCCTATAGAGGGAGCGGGAGCACCGG
GTGATCCGATGGATCAAGAGGATTTGGGACCTTGCTCCGAAGAGGAGGAATTCCAACAAGATGTAATG
GCAAGGGCCTGCCTGCAAAAGCTCAAAGGGACAACAGAACTCTTGCCCAAGAGGCCTCAACATCTGCC
CGGCCATCCAGGTTGGTATCGCAAACTCTGTCCAAGGCTGGACGATCCCGCCTGGACCCCGGGGCCCT
CCCTTCTGAGTCTGCAGATGAGAGTGACACCTAAGCTGATGGCACTTACTTGGGATGGGTTCCCTCTT
CACTATTCAGAGAGACACGGGTGGGGATATCTTGTCCCAGGTCGAAGGGACAATCTGGCGAAGCTTCC
CACAGGAACTACCTTGGAGAGTGCGGGCGTAGTATGTCCTTATCGAGCCATAGAAAGTCTGTATAGAA
AGCATTGCCTTGAACAAGGCAAACAACAACTCATGCCTCAGGAAGCCGGCCTCGCTGAAGAATTTCTT
CTTACTGATAACTCTGCTATCTGGCAAACAGTGGAGGAACTGGATTACCTCGAAGTCGAGGCCGAAGC
GAAGATGGAAAATTTGCGGGCGGCGGTCCCAGGTCAGCCCCTTGCTTTGACAGCACGGGGGGGCCCTA
AGGATACCCAGCCCAGCTATCATCACGGGAACGGTCCATATAATGATGTTGATATACCTGGTTGTTGG
TTTTTTAAACTTCCTCATAAAGATGGCAATTCATGTAACGTTGGATCCCCATTCGCAAAGGACTTCCT
CCCCAAAATGGAGGATGGGACGTTGCAAGCAGGTCCCGGTGGAGCTTCTGGGCCGCGAGCCCTGGAAA
TAAATAAGATGATTAGCTTCTGGAGGAATGCACACAAGCGCATTTCTTCACAGATGGTAGTGTGGCTG
CCTCGGAGTGCTTTGCCCAGGGCTGTGATCAGACACCCCGATTATGATGAAGAGGGACTGTACGGGGC
AATATTGCCCCAGGTGGTTACGGCTGGTACTATTACCCGCCGGGCAGTTGAGCCGACCTGGCTGACAG
CATCTAATGCCAGGCCTGATCGCGTGGGTTCTGAACTTAAAGCAATGGTCCAAGCTCCGCCTGGATAC
ACGCTTGTCGGCGCGGACGTGGACTCCCAGGAACTTTGGATAGCGGCTGTCCTTGGCGATGCACATTT
TGCAGGGATGCACGGGTGCACGGCTTTTGGCTGGATGACACTTCAGGGGAGGAAATCAAGGGGGACCG
ACCTGCACTCCAAGACCGCGACAACGGTAGGAATCTCAAGGGAACACGCTAAAATTTTCAATTATGGG
AGAATATATGGTGCCGGTCAACCGTTCGCTGAGCGCCTTCTGATGCAGTTTAACCATAGGCTGACACA
GCAGGAGGCAGCGGAGAAGGCGCAGCAGATGTACGCCGCAACTAAGGGTCTCAGATGGTATCGCCTCT
CAGACGAGGGCGAGTGGCTTGTCCGGGAATTGAACCTCCCGGTCGATCGGACGGAAGGTGGTTGGATT
AGTCTTCAGGATCTCAGAAAGGTGCAGCGGGAAACAGCACGCAAGTCTCAGTGGAAGAAATGGGAGGT
GGTCGCCGAGCGGGCATGGAAGGGTGGAACAGAATCCGAGATGTTTAACAAGCTGGAGAGCATAGCAA
CTAGTGACATCCCTAGGACACCGGTCTTGGGATGTTGCATTAGCCGGGCACTCGAGCCATCTGCCGTA
CAGGAAGAATTCATGACGTCACGGGTCAACTGGGTTGTCCAGTCTTCAGCCGTCGATTATCTGCATTT
GATGCTGGTCGCGATGAAGTGGCTCTTCGAAGAGTTTGCTATAGATGGCCGATTTTGCATCTCTATTC
ACGATGAGGTGCGCTACCTGGTAAGGGAAGAAGACCGATACCGAGCCGCCCTCGCCCTTCAGATAACA
AATCTGCTTACCAGGTGTATGTTTGCATACAAGCTGGGGTTGAACGACCTTCCCCAGTCCGTCGCTTT
CTTTTCAGCTGTTGATATAGATCGCTGCCTGAGAAAAGAGGTTACGATGGACTGCAAAACGCCCTCAA
ACCCCACTGGTATGGAGCGCAGATATGGCATCCCCCAAGGAGAAGCCCTCGACATATACCAGATAATT
GAGCTCACGAAGGGCAGCCTGGAGAAGAGATCCCAACCTGGCCCATAG

### Exemplary Recombinant AAV Compositions

The current disclosure provides for compositions containing a recombinant AAV containing a nucleic acid sequence that encodes a functional protein. Such proteins include, without limitation, TK2, dGK, p53R2, TP, SUCLA2, SUCLG1, POLG1, and a mitochondrial membrane protein MPV17. In some embodiments, the rAAV comprises a chicken beta-actin promoter. In some embodiments, the rAAV further comprises a CMV enhancer. In some embodiments, the rAAV further comprises ITRs. In some embodiments, the rAAV further comprises a 3′UTR. In some embodiments, the rAAV is AAV 2 or AAV9. In some embodiments, the transgene encodes a functional TK2 enzyme.

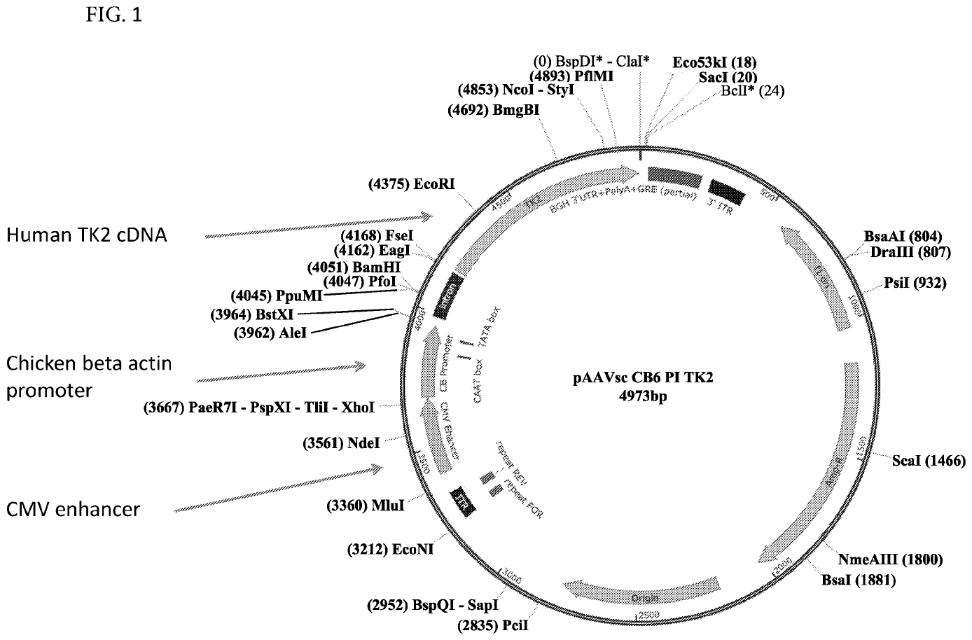

An exemplary rAAV-hTK2 vector comprising the human TK2 transgene, a chicken beta-actin promoter, a CMV IE enhancer, a BGH 3′UTR, a poly A signal, and a 5′ and 3′ITR is shown in FIG. 1.

Both an rAAV9-hTK2 and an rAAV2-hTK2 vector were used in the Examples. These constructs have the same ITR but different capsid protein sequences.

In some embodiments, the composition further comprises a pharmaceutical carrier.

In some embodiments, the composition comprises more than one rAAV comprising a transgene that encodes a functional protein including but not limited to TK2, dGK, p53R2, TP, SUCLA2, SUCLG1, POLG1, and a mitochondrial membrane protein MPV17.

### Routes of Administration and Dosing

The current invention provides rAAV vectors for use in methods of treating, preventing, and/or curing a disease or disorder characterized by unbalanced nucleotide pools, and/or restoring function of a protein that is dysfunctional in a disease or disorder characterized by unbalanced nucleotide pools, and/or alleviating in a subject at least one of the symptoms associated with unbalanced nucleotide pools. In some embodiments, methods involve administration of a rAAV vector that encodes one or more therapeutic peptides, polypeptides, shRNAs, microRNAs, or antisense nucleotides, in a pharmaceutically-acceptable carrier to the subject in an amount and for a period of time sufficient to treat, prevent and/or cure the disease or disorder characterized by unbalanced nucleotide pools in the subject having or suspected of having such a disorder.

Diseases or disorders characterized by unbalanced nucleotide pools that can be treated, prevented and/or cured by the method of the current invention include, but are not limited to, those characterized by mutations in the following genes: TK2; DGUOK; TYMP; RRM2B; SUCLA2; SUCLG1; and MPV17. The proteins encoded by these genes can be restored by the method of the invention and include but are not limited to TK2, dGK, p53R2, TP, SUCLA2, SUCLG1, POLG1, and a mitochondrial membrane protein MPV17.

The rAAVs may be delivered to a subject in compositions according to any appropriate methods known in the art. The rAAV, preferably suspended in a physiologically compatible carrier (e.g., in a composition), may be administered to a subject. In certain embodiments, compositions may comprise a rAAV alone, or in combination with one or more other viruses (e.g., a second rAAV encoding having one or more different transgenes). In one embodiment, a composition can comprise an rAAV9 vector comprising a nucleic acid sequence comprising a transgene encoding a functional protein including but not limited to TK2, dGK, p53R2, TP, SUCLA2, SUCLG1, POLG1, and a mitochondrial membrane protein MPV17.

In a further embodiment, a composition can comprise an rAAV2 vector comprising a nucleic acid sequence comprising a transgene encoding a functional protein including but not limited to TK2, dGK, p53R2, TP, SUCLA2, SUCLG1, POLG1, and a mitochondrial membrane protein MPV17.

In yet a further embodiment, a composition can comprise an rAAV9 vector comprising a nucleic acid sequence comprising a transgene encoding a functional TK2. In a further embodiment, a composition can comprise an rAAV2 vector comprising a nucleic acid sequence comprising a transgene encoding a functional TK2. In a further embodiment, a composition can comprise an rAAV9 vector comprising a nucleic acid sequence comprising a transgene encoding TK2 and an rAAV2 vector comprising a nucleic acid sequence comprising a transgene encoding TK2.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pH adjustment, and salt concentration adjustment (see, e.g., Wright, et al., Molecular Therapy 12:171-178 (2005).

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

rAAVS are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, and other parental routes of administration. Routes of administration may be combined, if desired. The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

The present invention provides stable pharmaceutical compositions comprising rAAV virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration; the level of gene or RNA expression required to achieve a therapeutic effect; the specific disease or disorder being treated; and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target desired tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby, etc. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient or carriers. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions.

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

A preferred route of administration of the AAVs is intravenously.

A preferred dose ranges from about 1×10¹⁰ to about 8×10¹¹, from about 2×10¹⁰ to about 6×10¹¹, from about 4×10¹⁰ to about 4×10¹¹ genome or viral copy (vc) total administration. A preferred dose is about 4×10¹¹ genome or viral copy (vc) total administration of rAAV.

If more than one rAAV is used a preferred total dose of vector ranges from about 1×10¹⁰ to about 6×10¹¹ from about 2×10¹⁰ to about 5×10¹¹, from about 1×10¹⁰ to about 4×10¹¹ genome or viral copy (vc) total administration. A preferred dose of total vector is about 3×10¹¹. The AAV can be administered in equal amounts, e.g., ratio of 50/50, or in or in ratios of about 5/95, 10/90, 15/85, 20/80, 25/75, 30/70, 35/65, 40/60, 45/55, 55/45, 60/40, 65/35, 70/30, 75/25, 80/20, 85/15, 90/10, and 95/5.

Doses can be adjusted to optimize the effects in the subject. Additionally, a subject can be monitored for improvement of their condition prior to increasing the dosage. A subject's response to the therapeutic administration of the rAAV can be monitored by observing a subject's muscle strength and control, and mobility as well as changes in height and weight. If one or more of these parameters increase after the administration, the treatment can be continued. If one or more of these parameters stays the same or decreases, the dosage can be increased.

### Pharmacological Therapy—Methods of Administration and Dosing

The present invention encompasses the administration of deoxynucleosides, and/or deoxyribonucleoside monophosphates, in combination with the administration of a composition containing a transgene encoding a protein described herein.

Most preferred methods of administration are oral, intrathecal and parental including intravenous. The deoxynucleosides or deoxyribonucleoside monophosphates must be in the appropriate form for administration of choice.

Deoxynucleosides or deoxyribonucleoside monophosphates are easily dissolved in liquid are easily dissolved in liquid (such as water, formula or milk) whereas the free acid form does not readily dissolve in liquid.

Such pharmaceutical compositions comprising one of more deoxynucleosides or deoxyribonucleoside monophosphates for administration may comprise a therapeutically effective amount of the deoxynucleosides or deoxyribonucleoside monophosphates and a pharmaceutically acceptable carrier. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Oral administration is a preferred method of administration. The deoxynucleosides or deoxyribonucleoside monophosphates can be added to any form of liquid a patient would consume including but not limited to, milk, both cow's and human breast, infant formula, and water.

Additionally, pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

In order to overcome any issue of the deoxynucleosides or deoxyribonucleoside monophosphates crossing the blood/brain barrier, intrathecal administration is an additional preferred form of administration (Galbiati, et al. 2006; Gotz, et al. 2008). Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Additionally, since some patients may be receiving enteral nutrition by the time the deoxynucleoside or deoxyribonucleoside monophosphates treatment begins, the dNs or dNMPs can be administered through a gastronomy feeding tube or other enteral nutrition means.

Further methods of administration include mucosal, such as nasal, sublingual, vaginal, buccal, or rectal; or transdermal administration to a subject.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for transdermal administration may be provided as discrete patches intended to remain in intimate contact with the epidermis of the recipient over a prolonged period of time.

The deoxynucleoside therapy comprises the administration of one or more deoxynucleosides chosen from the group consisting of deoxythymidine (dT), deoxycytidine (dC), deoxyadenosine (dA) and deoxyguanosine (dG).

A skilled practitioner can determine which deoxynucleosides are beneficial based upon the deficiency. It is also within the skill of the art for the practitioner to determine if mixtures of the deoxynucleosides should be administered and in what ratio. If two deoxynucleosides are to be administered, they can be in a ratio of 50/50 of each deoxynucleoside, e.g., dC and dT, or in ratios of about 5/95, 10/90, 15/85, 20/80, 25/75, 30/70, 35/65, 40/60, 45/55, 55/45, 60/40, 65/35, 70/30, 75/25, 80/20, 85/15, 90/10, and 95/5.

By way of example, dT and dC are administered in mixture of equal amounts for TK2 deficiency.

The deoxyribonucleoside monophosphates therapy comprises the administration of one or more deoxyribonucleoside monophosphates chosen from the group consisting of TMP, dCMP, dAMP, and dGMP. If two deoxyribonucleoside monophosphates, ratios can be determined.

By way of example, TMP and dCMP are administered in mixture of equal amounts for TK2 deficiency.

Selection of a therapeutically effective dose will be determined by the skilled artisan considering several factors, which will be known to one of ordinary skill in the art. Such factors include the particular form of the deoxynucleoside or deoxyribonucleoside monophosphate, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

A preferred dose ranges from about 100 mg/kg/day to about 1,000 mg/kg/day. A further preferred dose ranges from about 200 mg/kg/day to about 800 mg/kg/day. A further preferred dose ranges from about 250 mg/kg/day to about 400 mg/kg/day. These dosage amounts are of individual deoxynucleosides or deoxyribonucleoside monophosphates or of a composition with a mixture of more than one deoxynucleosides, e.g., dT and dC or deoxyribonucleoside monophosphates. For example, a dose can comprise 400 mg/kg/day of dT alone. In a further example, a dose can comprise a mixture of 200 mg/kg/day of dT and 200 mg/kg/day of dC. In a further example, a dose can comprise 400 mg/kg/day of a mixture of dT and dC.

Administration of the deoxynucleosides or deoxyribonucleoside monophosphates can be once a day, twice a day, three times a day, four times a day, five times a day, up to six times a day, preferably at regular intervals. For example, when the deoxynucleosides are administered four times daily, doses would be at 8:00 AM, 12:00 PM, 4:00 PM, and 8:00 PM.

Doses can also be lowered if being administered intravenously or intrathecally. Preferred dose ranges for such administration are from about 50 mg/kg/day to about 500 mg/kg/day.

As shown in the '092 application, doses can be adjusted to optimize the effects in the subject. For example, the deoxynucleosides can be administered at 100 mg/kg/day to start, and then increased over time to 200 mg/kg/day, to 400 mg/kg/day, to 800 mg/kg/day, up to 1000 mg/kg/day, depending upon the subject's response and tolerability.

A subject can be monitored for improvement of their condition prior to increasing the dosage. A subject's response to the therapeutic administration of the deoxynucleosides or deoxyribonucleoside monophosphates can be monitored by observing a subject's muscle strength and control, and mobility as well as changes in height and weight. If one or more of these parameters increase after the administration, the treatment can be continued. If one or more of these parameters stays the same or decreases, the dosage of the deoxynucleosides can be increased.

As shown in the prior patent applications, the pharmacological therapy is well tolerated. Any observed adverse effects were minor and were mostly diarrhea, abdominal bloating and other gastrointestinal manifestations. A subject can also be monitored for any adverse effects, such as gastrointestinal intolerance, e.g., diarrhea. If one or more adverse effects are observed after administration, then the dosage can be decreased. If no such adverse effects are observed, then the dosage can be increased. Additionally, once a dosage is decreased due to the observation of an adverse effect, and the adverse effect is no longer observed, the dosage can be increased.

The deoxynucleosides or deoxyribonucleoside monophosphates can also be co-administered with other agents. Such agents would include therapeutic agents for treating the symptoms of the particular form of MDS. In particular, for TK2 deficiency, the dT and dC can be co-administered with an inhibitor of ubiquitous nucleoside catabolic enzymes, including but not limited to enzyme inhibitors such as tetrahydrouridine (inhibitor of cytidine deaminase) and immucillin H (inhibitor of purine nucleoside phosphorylase) and tipiracil (inhibitor of thymidine phosphorylase). Such inhibitors are known and used in the treatment of some cancers.

### Kits

The present invention also provides kits comprising the components of the combinations of the invention in kit form. A kit of the present invention includes one or more components including, but not limited to, viral vectors (e.g., AAV vectors) and/or pharmacological agents (e.g., deoxynucleosides and/or nucleoside monophosphates) described herein. Kits may further include a pharmaceutically acceptable carrier, as discussed herein. The viral vector or pharmacological agent can be formulated as a pure composition or in combination with a pharmaceutically acceptable carrier, in a pharmaceutical composition.

In some embodiments, a kit includes an AAV vector containing a transgene described herein in one container (e.g., in a sterile glass or plastic vial).

In some embodiments, a kit includes an AAV vector containing a transgene described herein in one container (e.g., in a sterile glass or plastic vial) and a second AAV vector encoding a transgene described herein in another container (e.g., in a sterile glass or plastic vial).

In some embodiments, a kit includes an AAV vector containing a transgene described herein in one container (e.g., in a sterile glass or plastic vial) and a pharmacological agent in another container (e.g., in a sterile glass or plastic vial).

In some embodiments, a kit includes an AAV2 vector containing a transgene described herein in one container (e.g., in a sterile glass or plastic vial) and a pharmacological agent in another container (e.g., in a sterile glass or plastic vial).

In some embodiments, a kit includes an AAV9 vector containing a transgene described herein in one container (e.g., in a sterile glass or plastic vial) and a pharmacological agent in another container (e.g., in a sterile glass or plastic vial).

In some embodiments, a kit includes an AAV vector encoding TK2, dGK, p53R2, TP, SUCLA2, SUCLG1, POLG1, and/or a mitochondrial membrane protein MPV17, or a pharmaceutical composition thereof in one container (e.g., in a sterile glass or plastic vial).

If the kit includes one or more pharmaceutical compositions for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Exemplary Embodiments of the Invention Include

1. A method of preventing or treating thymidine kinase 2 (TK2) deficiency in a subject in need thereof comprising administering to the subject a therapeutically effective amount of a composition comprising a recombinant adeno-associated vector (rAAV) comprising a nucleic acid sequence comprising a transgene encoding a functional TK2 protein.
2. The method of embodiment 1, wherein the rAAV is chosen from the group consisting of AAV9 and AAV2.
3. The method of embodiment 1, wherein the subject is a mammal.
4. The method of embodiment 1, wherein the subject is a human.
5. The method of embodiment 1, wherein the composition is a pharmaceutical composition.
6. The method of embodiment 1, wherein the composition is the first composition comprising a first rAAV, wherein the first rAAV is AAV9 and further comprising administering a therapeutically effective amount of a second composition comprising a second rAAV comprising a nucleic acid sequence comprising a transgene encoding a functional TK2 protein, wherein the second rAAV is AAV2 and is administered at a time point after the administration of the first rAAV.
7. The method of embodiment 6, wherein the second composition is a pharmaceutical composition.
8. The method of embodiment 6, wherein the first composition is administered as soon as the subject is diagnosed with TK2 deficiency or suspected of having TK2 deficiency, and is continued to be administered during the administration of the second composition.
9. The method of embodiment 6, wherein the second composition is administered within days of the first composition.
10. The method of embodiment 6, wherein the second composition is administered within weeks of the first composition.
11. The method of embodiment 6, wherein the second composition is administered at least a month after the first composition.
12. The method of embodiment 6, further comprising administering a therapeutically effective amount of a third composition comprising a pharmacological agent chosen from the group consisting of deoxycytidine (dC), deoxythymidine (dT), and mixtures thereof.
13. The method of embodiment 12, wherein the third composition is administered at the same time point as the second composition.
14. The method of embodiment 12, wherein the third composition is administered at a later time point as the second composition.
15. The method of embodiment 12, wherein the therapeutically effective amount of the third composition is between about 100 mg/kg/day and about 1000 mg/kg/day.
16. The method of embodiment 12, wherein the therapeutically effective amount of the third composition is between about 200 mg/kg/day and about 800 mg/kg/day.
17. The method of embodiment 12, wherein the therapeutically effective amount of the third composition is between about 250 mg/kg/day and about 400 mg/kg/day.
18. The method of embodiment 12, wherein the third composition is administered once daily, twice daily, three times daily, four times daily, five times daily or six times daily.
19. The method of embodiment 12, wherein the third composition is administered orally, intrathecally, enterally, or intravenously.
20. The method of embodiment 19, wherein the third composition is administered orally and comprises deoxynucleoside mixed with cow's milk, human breast milk, infant formula or water.
21. A method of preventing or treating thymidine kinase 2 (TK2) deficiency in a subject in need thereof comprising administering to the subject a therapeutically effective amount of a first composition comprising a pharmacological agent, and further administering a therapeutically effective amount of a second composition comprising a recombinant adeno-associated vector (rAAV) comprising a nucleic acid sequence comprising a transgene encoding a functional TK2 protein.
22. The method of embodiment 21, wherein the pharmacological agent is chosen from the group consisting of deoxycytidine (dC), deoxythymidine (dT), and mixtures thereof; and deoxycytidine monophosphate (dCMP), deoxythymidine monophosphate (dTMP), and mixtures thereof.
23. The method of embodiment 21, wherein the rAAV is AAV9.
24. The method of embodiment 21, wherein the second composition is a pharmaceutical composition.
25. The method of embodiment 21, wherein the first composition is administered as soon as the subject is diagnosed with TK2 deficiency or suspected of having TK2 deficiency, and is continued to be administered during the administration of the second composition.
26. The method of embodiment 21, wherein the second composition is administered within days of the first composition.
27. The method of embodiment 21, wherein the second composition is administered within weeks of the first composition.
28. The method of embodiment 21, wherein the second composition is administered at least a month after the first composition.
29. The method of embodiment 21, wherein the therapeutically effective amount of the first composition is between about 100 mg/kg/day and about 1000 mg/kg/day.
30. The method of embodiment 21, wherein the therapeutically effective amount of the first composition is between about 200 mg/kg/day and about 800 mg/kg/day.
31. The method of embodiment 21, wherein the therapeutically effective amount of the first composition is between about 250 mg/kg/day and about 400 mg/kg/day.
32. The method of embodiment 21, wherein the first composition is administered once daily, twice daily, three times daily, four times daily, five times daily or six times daily.
33. The method of embodiment 21, wherein the first composition is administered orally, intrathecally, enterally, or intravenously.
34. The method of embodiment 33, wherein the first composition is administered orally and comprises the pharmacological agent mixed with cow's milk, human breast milk, infant formula or water.
35. The method of embodiment 21, further comprising the administration of a third composition comprising a second rAAV comprising a recombinant adeno-associated vector (rAAV) comprising a nucleic acid sequence comprising a transgene encoding a functional TK2 protein wherein the second rAAV is AAV2.
36. A method of restoring thymidine kinase 2 enzyme activity in a subject in need thereof comprising administering to the subject a therapeutically effective amount of a composition comprising a recombinant adeno-associated vector (rAAV) comprising a nucleic acid sequence comprising a transgene encoding a functional TK2 protein.
37. The method of embodiment 36, wherein the rAAV is chosen from the group consisting of AAV9 and AAV2.
38. The method of embodiment 36, wherein the subject is a mammal.
39. The method of embodiment 36, wherein the subject is a human.
40. The method of embodiment 36, wherein the composition is a pharmaceutical composition.
41. The method of embodiment 36, wherein the composition is the first composition comprising a first rAAV, wherein the first rAAV is AAV9 and further comprising administering a therapeutically effective amount of a second composition comprising a second rAAV comprising a nucleic acid sequence comprising a transgene encoding a functional TK2 protein, wherein the second rAAV is AAV2 and is administered at a time point after the administration of the first rAAV.
42. The method of embodiment 41, wherein the second composition is a pharmaceutical composition.
43. The method of embodiment 41, wherein the first composition is administered as soon as the subject is diagnosed with TK2 deficiency or suspected of having TK2 deficiency, and is continued to be administered during the administration of the second composition.
44. The method of embodiment 41, wherein the second composition is administered within days of the first composition.
45. The method of embodiment 41, wherein the second composition is administered within weeks of the first composition.
46. The method of embodiment 41, wherein the second composition is administered at least a month after the first composition.
47. The method of embodiment 41, further comprising administering a therapeutically effective amount of a third composition comprising a pharmacological agent chosen from the group consisting of deoxycytidine (dC), deoxythymidine (dT), and mixtures thereof.
48. The method of embodiment 47, wherein the third composition is administered at the same time point as the second composition.
49. The method of embodiment 47, wherein the third composition is administered at a later time point as the second composition.
50. The method of embodiment 47, wherein the therapeutically effective amount of the third composition is between about 100 mg/kg/day and about 1000 mg/kg/day.
51. The method of embodiment 47, wherein the therapeutically effective amount of the third composition is between about 200 mg/kg/day and about 800 mg/kg/day.
52. The method of embodiment 47, wherein the therapeutically effective amount of the third composition is between about 250 mg/kg/day and about 400 mg/kg/day.
53. The method of embodiment 47, wherein the third composition is administered once daily, twice daily, three times daily, four times daily, five times daily or six times daily.
54. The method of embodiment 47, wherein the third composition administered orally, intrathecally, enterally, or intravenously.
55. The method of embodiment 54, wherein the third composition is administered orally and comprises deoxynucleoside mixed with cow's milk, human breast milk, infant formula or water.
56. A method of restoring thymidine kinase 2 enzyme activity in a subject in need thereof comprising administering to the subject a therapeutically effective amount of a first composition comprising a pharmacological agent, and further administering a therapeutically effective amount of a second composition comprising a recombinant adeno-associated vector (rAAV) comprising a nucleic acid sequence comprising a transgene encoding a functional TK2 protein.
57. The method of embodiment 56, wherein the pharmacological agent is chosen from the group consisting of deoxycytidine (dC), deoxythymidine (dT), and mixtures thereof, and deoxycytidine monophosphate (dCMP), deoxythymidine monophosphate (dTMP), and mixtures thereof.
58. The method of embodiment 56, wherein the rAAV is AAV9.
59. The method of embodiment 56, wherein the second composition is a pharmaceutical composition.
60. The method of embodiment 56, wherein the first composition is administered as soon as the subject is diagnosed with TK2 deficiency or suspected of having TK2 deficiency, and is continued to be administered during the administration of the second composition.
61. The method of embodiment 56, wherein the second composition is administered within days of the first composition.
62. The method of embodiment 56, wherein the second composition is administered within weeks of the first composition.
63. The method of embodiment 56, wherein the second composition is administered at least a month after the first composition.
64. The method of embodiment 56, wherein the therapeutically effective amount of the first composition is between about 100 mg/kg/day and about 1000 mg/kg/day.
65. The method of embodiment 56, wherein the therapeutically effective amount of the first composition is between about 200 mg/kg/day and about 800 mg/kg/day.
66. The method of embodiment 56, wherein the therapeutically effective amount of the first composition is between about 250 mg/kg/day and about 400 mg/kg/day.
67. The method of embodiment 56, wherein the first composition is administered once daily, twice daily, three times daily, four times daily, five times daily or six times daily.
68. The method of embodiment 56, wherein the first composition administered orally, intrathecally, enterally, or intravenously.
69. The method of embodiment 56, wherein the first composition is administered orally and comprises the pharmacological agent mixed with cow's milk, human breast milk, infant formula or water.
70. The method of embodiment 56, further comprising the administration of a third composition comprising a second rAAV comprising a recombinant adeno-associated vector (rAAV) comprising a nucleic acid sequence comprising a transgene encoding a functional TK2 protein wherein the second rAAV is AAV2.
71. A method of preventing or treating a disease or disorder characterized by unbalanced nucleotide pools in a subject in need thereof comprising administering to the subject a therapeutically effective amount of a composition comprising a recombinant adeno-associated vector (rAAV) comprising a nucleic acid sequence comprising a transgene chosen from the group consisting of TK2; DGUOK; TYMP; RRM2B; SUCLA2; SUCLGJ; and MPV17.
72. A method of preventing or treating a disease or disorder characterized by unbalanced nucleotide pools in a subject in need thereof comprising administering to the subject a therapeutically effective amount of a first composition comprising a pharmacological agent, and further administering a therapeutically effective amount of a second composition comprising a recombinant adeno-associated vector (rAAV) comprising a nucleic acid sequence comprising a transgene chosen from the group consisting of TK2; DGUOK; TYMP; RRM2B; SUCLA2; SUCLGJ; and MPV17.
73. The method of embodiment 72, wherein the pharmacological agent is chosen from the group consisting of deoxycytidine (dC), deoxythymidine (dT), deoxyadenosine (dA), deoxyguanosine (dG), and mixtures thereof; and deoxycytidine monophosphate (dCMP), deoxythymidine monophosphate (dTMP), deoxyadenosine monophosphate (dAMP), deoxyguanosine monophosphate (dGMP) and mixtures thereof.
74. A method of restoring enzyme activity in a disease or disorder characterized by unbalanced nucleotide pools in a subject in need thereof comprising administering to the subject a therapeutically effective amount of a composition comprising a recombinant adeno-associated vector (rAAV) comprising a nucleic acid sequence comprising a transgene chosen from the group consisting of TK2; DGUOK; TYMP; RRM2B; SUCLA2; SUCLGJ; and MPV17.
75. A method of restoring enzyme activity in a disease or disorder characterized by unbalanced nucleotide pools in a subject in need thereof comprising administering to the subject a therapeutically effective amount of a first composition comprising a pharmacological agent, and further administering a therapeutically effective amount of a second composition comprising a recombinant adeno-associated vector (rAAV) comprising a nucleic acid sequence comprising a transgene chosen from the group consisting of TK2; DGUOK; TYMP; RRM2B; SUCLA2; SUCLGJ; and MPV17.
76. The method of embodiment 75, wherein the pharmacological agent is chosen from the group consisting of deoxycytidine (dC), deoxythymidine (dT), deoxyadenosine (dA), deoxyguanosine (dG), and mixtures thereof; and deoxycytidine monophosphate (dCMP), deoxythymidine monophosphate (dTMP), deoxyadenosine monophosphate (dAMP), deoxyguanosine monophosphate (dGMP) and mixtures thereof.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods

### Mouse Model of TK2 Deficiency

A homozygous Tk2 H126N knock-in mutant (Tk2^{-/-}) mouse that manifests a phenotype strikingly similar to the human infantile encephalomyopathy has been previously reported (Akman, et al. 2008). Between postnatal day 10 and 13, Tk2^{-/-} mice rapidly develop fatal encephalomyopathy characterized by decreased ambulation, unstable gait, coarse tremor, growth retardation, and rapid progression to early death at age 14 to 16 days. Molecular and biochemical analyses of the mouse model demonstrated that the pathogenesis of the disease is due to loss of enzyme activity and ensuing dNTP pool imbalances with decreased dTTP levels in brain and both dTTP and dCTP levels in liver, which, in turn, produces mtDNA depletion and defects of respiratory chain enzymes containing mtDNA-encoded subunits, most prominently in the brain and spinal cord.

All experiments were performed according to a protocol approved by the Institutional Animal Care and Use Committee of the Columbia University Medical Center, and were consistent with the National Institutes of Health Guide for the Care and Use of Laboratory Animals. Mice were housed and bred according to international standard conditions, with a 12-hour light, 12-hour dark cycle, and sacrificed at 4, 13, and 29 days of age.

Organs (brain, spinal cord, liver, heart, kidney, quadriceps muscle, lung, and gastrointestinal tract) were removed and either frozen in the liquid phase of isopentane, pre-cooled near its freezing point (−160° C.) with dry ice or fixed in 10% neutral buffered formalin and embedded in paraffin using standard procedures. Paraffin embedded tissue were then stained with hematoxylin and eosin (H&E) for morphological study or processed for immunostaining studies with GFAP, COX I, or complex I. Both heterozygous and homozygous wild type mice were considered as control group (Tk2^{-/-}) since no clinical and biochemical difference were previously described (Akman, et al. 2008; Dorado, et al. 2011).

hTK2 Construct

The vector comprising the human TK2 gene (pAAVsc CB6 PI TK2 is shown in FIG. 1. Key components of the construct include the human TK2 cDNA as well as 5′ITR, CMV IE enhancer, chicken beta actin promoter, BGH 3′UTR, Poly A signal, GRE, and 3′ ITR.

This vector was encapsulated in both AAV9 and AAV2 capsids for the subsequent experiments.

### Gene Therapy Administration

Based upon previous results using an AAV9-GFP vector in wild-type mice and analyzing the distribution of the vector showed that AAV9 virus does not target the brain when administered I.P. (Results not shown.) Based upon this work, the administration of the AAV9-hTK2 construct was done by intravenous (IV) injection.

The AAV9-hTK2 was administered via IV with a retro-orbital injection of a total volume of 35 μl of PBS 1X containing about 4.2×10¹¹ or 4.2×10¹⁰ genome or vector copies at postnatal day 1.

Alternatively, AAV9-hTK2 was administered via IV with a retro-orbital injection of 35 μL of PBS 1X containing about 2.1×10¹¹ vector copies at postnatal day 1, followed by administration of 100 μL of AAV2-hTK2 via IV with a tail vein injection containing about 1.05×10¹¹ vector copies at day 29 and with and without supplementation with 520 mg/kg/day of oral dC+dT (in drinking water) from day 21 assuming a water consumption of 4 ml/day/mouse.

### Pharmacological Treatment Administration and Experimental Plan

Deoxycytidine (dC) and deoxythymidine (dT) are administered in 50 μl of Esbilac milk formula for small pets (Pet-Ag) by daily oral gavage to Tk2^{-/-} and aged matched control wild-type (Tk2^{-/-}) using a dose of 520 mg/kg/day, from postnatal day 4 to 29 days. At age postnatal 21 days, mice are separated from the mother and the treatment was continued by administration of dC and dT in drinking water assuming a water consumption of 4 ml/day/mouse. A negative control group of untreated Tk2 mutant and control wild-type mice are weighed and observed closely for comparison.

Deoxycytidine monophosphate (dCMP) and deoxythymidine monophosphate (TMP) (Hongene Biotech, Inc.) are administered in 50 μl of Esbilac milk formula for small pets (Pet-Ag) by daily oral gavage to Tk2 H126N knock-in mice (Tk2^{-/-}) and aged-matched control wild-type (Tk2^{-/-}) using 2 doses, 200 mg/kg/day and 400 mg/kg/day, from postnatal day 4 to 29 days. At age 29 days, mice are separated from the mother and the treatment is continued by administration of dCMP and TMP in drinking water using a dose of 400 mg/kg/day.

### Phenotype Assessment

Body weight was assessed daily, since it has been previously observed that incapacity of gaining weight is the first sign of disease (Akman, et al. 2008).

To define the degree of safety and efficacy of the gene therapy, survival time, age-at-onset of disease, type and severity of symptoms, occurrence of side effects, and proportion of treatment termination due to adverse events in treated and untreated Tk2 mice were compared. General behavior, survival time, and body weights of the mice were assessed daily beginning at postnatal day 4.

### TK2 Enzyme Measurement

The activity of TK2 enzyme was measured as previously described in Franzolin, et al. 2006.

mtDNA Copy Number Measurement

mtDNA copy number was measured as previously described in Spinazzola, et al. 2006/

### Mitochondrial Respiratory Chain Enzyme Activities

Mitochondrial RC enzymes analysis was performed in cerebrum tissue as previously described (DiMauro, et al. 1987; Birch-Machin, et al. 1994; Quinzii, et al. 2013).

### Statistical Methods

Data are expressed as the mean±SD of at least 3 experiments per group. Gehan-Breslow-Wilcoxon test was used to compare the survival proportion of each group of mice. A p-value of <0.05 was considered to be statistically significant.

### Example 2—Administration of AAV9-hTK2 Resulted in Prolonged Lifespan

The mutant mice described in Example 1 were administered via IV with a retro-orbital injection at postnatal day 1 with the AAV9-hTK2 construct (containing about 4.2×10¹¹ or 4.2×10¹⁰ genome or vector copies) also described in Example 1.

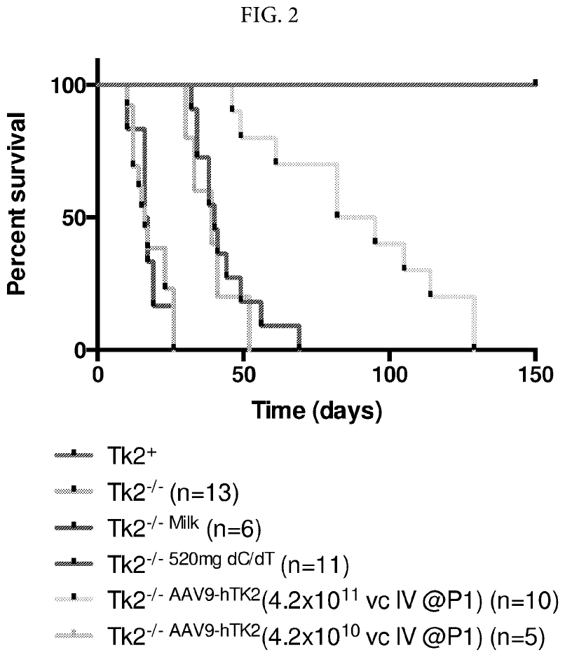

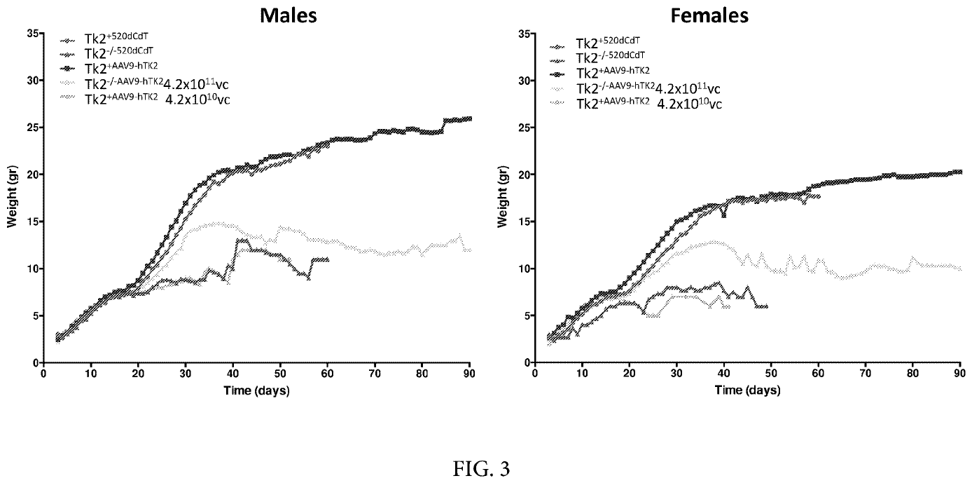

Treatment with AAV9-hTK2 resulted in a dose-dependent response with administration of 4.2×10¹⁰ vc prolonging lifespan up to 3-fold (average of 39 days), similar to 520 mg/kg/day of dC+dT therapy. Administration of 4.2×10¹¹ vc prolonged lifespan up to 6-fold (average of 89 days) with a maximum of 129 days. See FIG. 2

### Example 3—Administration of AAV9-hTK2 Resulted in Increased Growth, Strength and Motor Function in Mutant Mice

The mice described in Example 1 were treated as described in Example 1 with the AAV9-hTK2 construct also described in Example 1.

The Tk2^{-/-} mice treated with the lower dose of AAV9-hTK2 (4.2×10¹⁰vc) grew at the same rate as those Tk2^{-/-} mice treated with 520 mg/kg/day of dC+dT. They also grew at the same rate as Tk2⁺ mice until postnatal day 20, when they reached a plateau.

The Tk2^{-/-} mice treated with the higher dose of AAV9-hTK2 (4.2×10¹¹vc) grew at the same rate as Tk2⁺ mice until P30.

No differences were found between untreated Tk2⁺, and Tk2⁺ treated with the higher dose of AAV9-hTK2 (4.2×10¹¹vc). See FIG. 3.

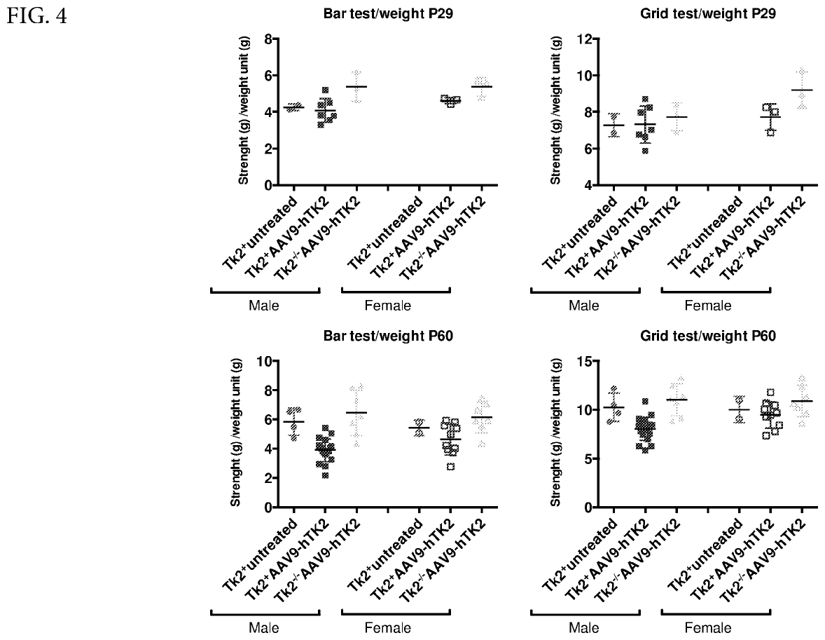

The Tk2^{-/-} mice treated with 4.2×10¹¹ vc at P1 showed no differences in strength compared to untreated or AAV9-hTK2 treated wild-type mice. Strength was normalized by weight and measured at P29 and P60. See FIG. 4.

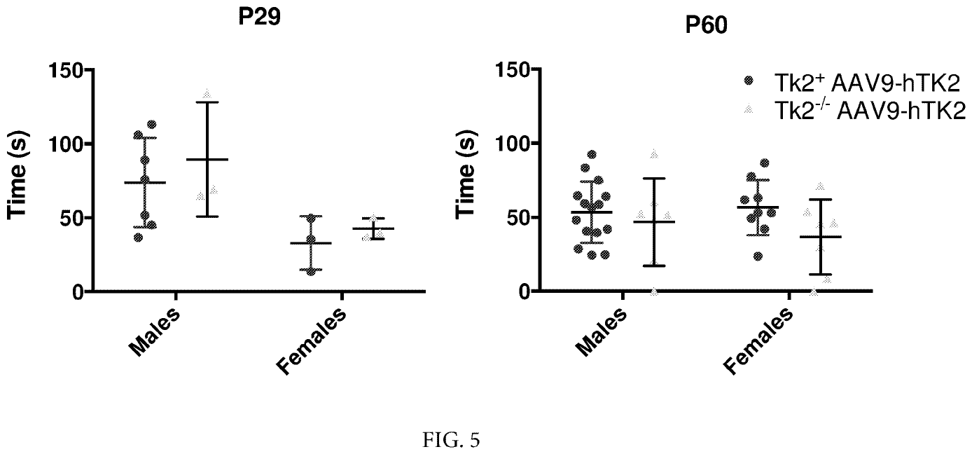

Rotarod testing, an outcome measure believed to model motor behavior defects, i.e. motor phenotypes, observed in human patients for assessing the motor function, was performed at postnatal day 29 and 60, and showed no differences between the treated mutant mice and treated wild-type mice, although two mutant, one male and one female, could not perform the test because of weakness and lack of balance. See FIG. 5.

Wild-type mice treated with the AAV9-hTK2 showed no adverse side-effects related to the therapy and were overall healthy.

The results regarding strength and motor function appear to rule out myopathy in the mutant mice and suggest that reduced weight showed in Tk2^{-/-} may be caused by a non-muscle related health condition.

### Example 4—Treatment with AAV9-Tk2 Restored TK2 Enzyme Activity in Most Tissues

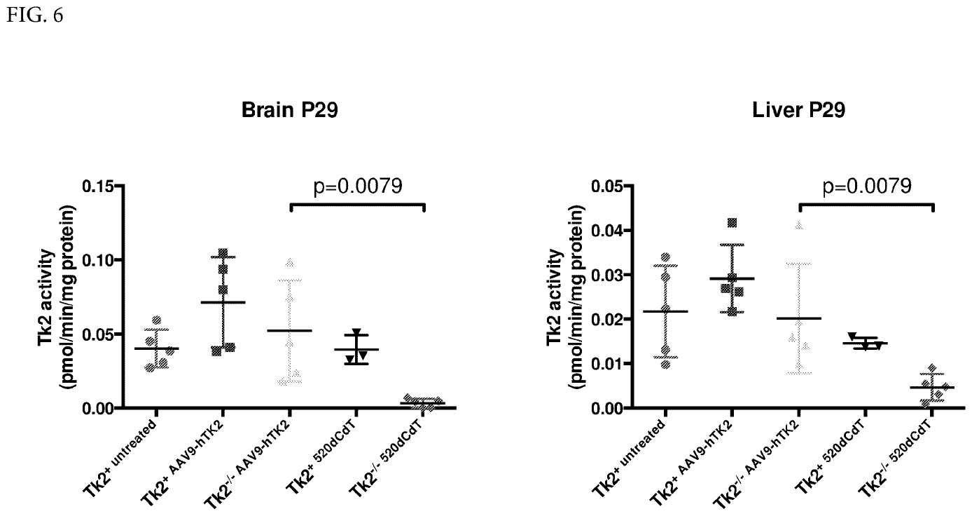

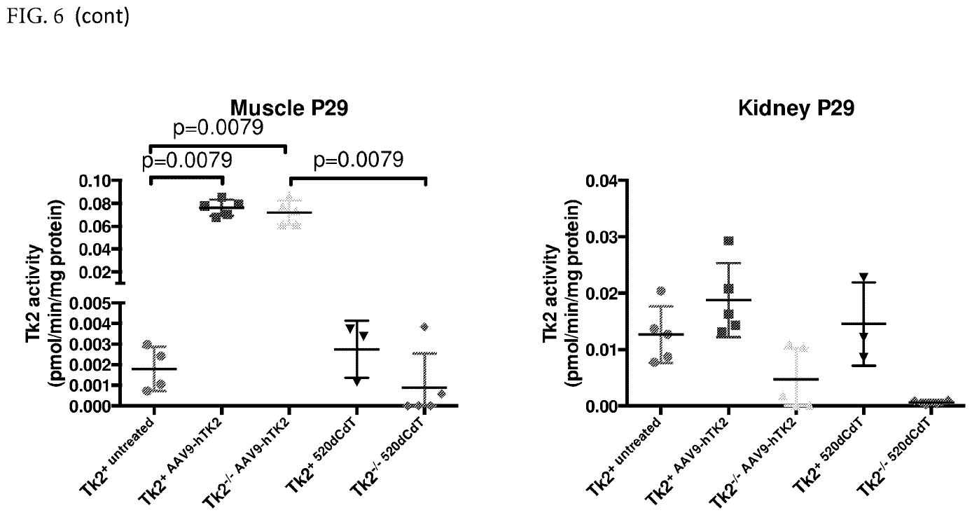

Using the methods described in Example 1, the activity of the TK2 enzyme was measured at P29 and shown to be restored in mutant mice treated with 4.2×10¹¹vc of AAV9-hTK2 at P1 in brain and liver to levels similar to those found in untreated wild-type mice. In muscle, AAV9-hTK2 increased 40-fold the TK2 activity as compared to untreated wild-type mice. However, the administration of the AAV9-hTK2 showed a poor efficiency restoring TK2 activity in kidney. See FIG. 6.

### Example 5—Treatment with AAV9-hTk2 Increased the Expression of hTK2 in Most Tissues

Human TK2 was measured as a percentage with respect of mRNA expression of mouse TK2 as described in Example 1 in wild-type mice treated with AAV9-hTK2 (4×10¹¹ vc) at 1 month, 2 months, and 6 months in brain, liver, muscle, and kidney tissue.

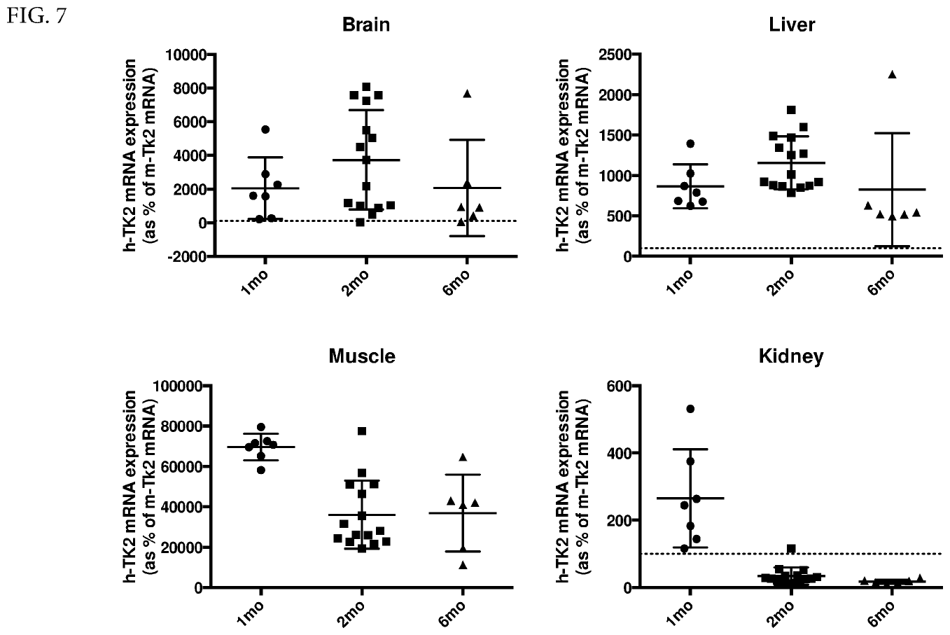

Human TK2 was expressed in brain, liver and muscle above 100% of the expression of mouse TK2 on target tissue, even in wild-type mice after 6 months of treatment. In contrast, expression of human TK2 in kidney is very low 2 months after the treatment. See FIG. 7.

### Example 6—Treatment with AAV9-hTK2 Rescued mtDNA in Most Tissues

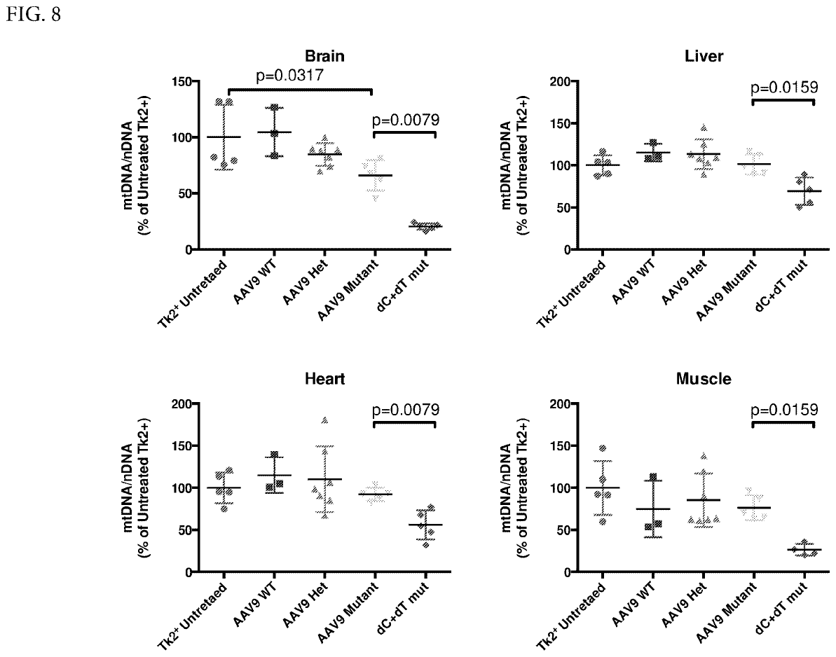

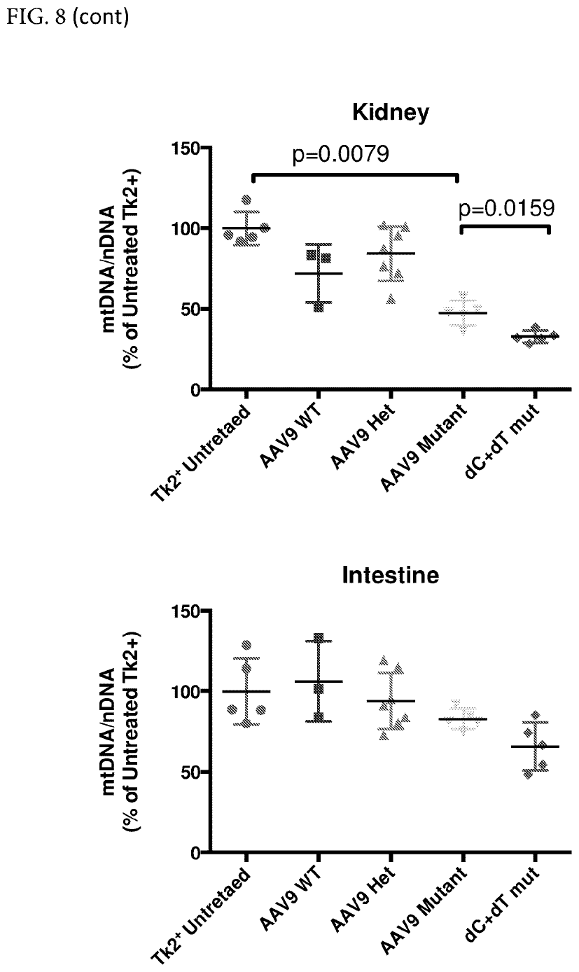

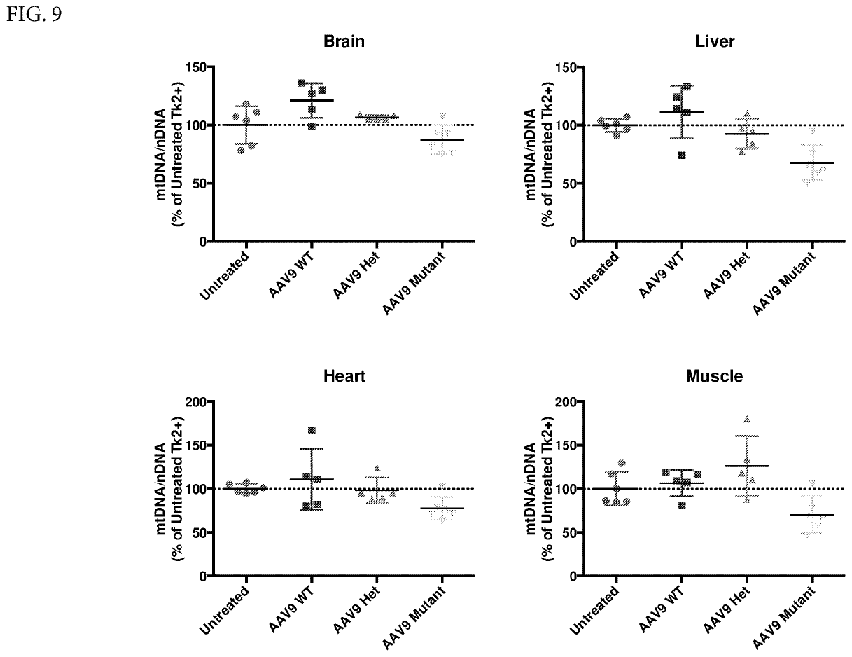

mtDNA copy number was measured as described in Example 1. At postnatal day 29, Tk2^{-/-} mice treated with AAV9-hTK2 (4×10¹¹ vc) at postnatal day 1 had signs of severe mtDNA depletion only in kidney tissue and mtDNA was partially rescued in brain, heart, liver, intestine and muscle (FIG. 8).

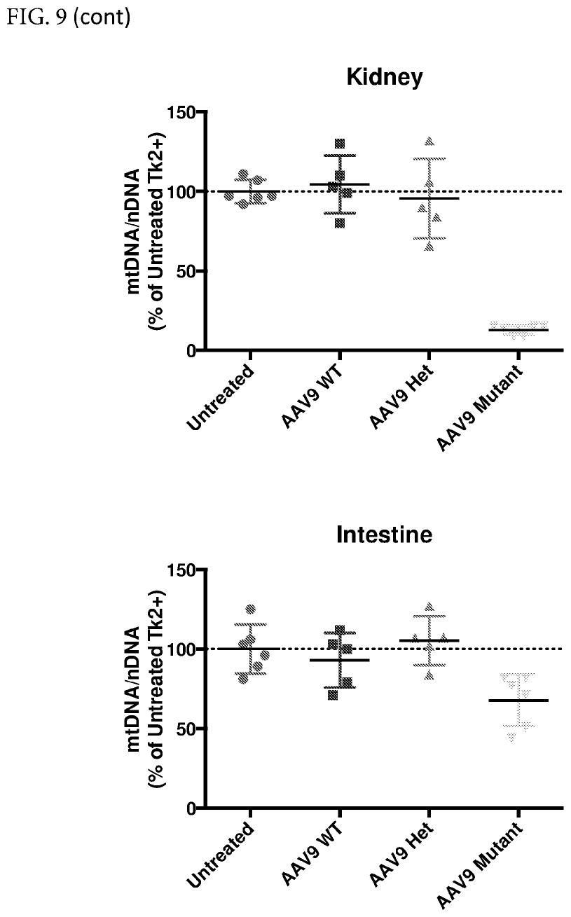

At postnatal day 60, there was very mild mtDNA depletion (65-80% of untreated wild-type mice) in liver, heart, muscle and intestine tissue of AAV9-hTK treated mutant mice. mtDNA in brain tissue of treated mutant mice was 87% that of untreated wild-type mice. This showed a rescue of mtDNA in most tissues of AAV9-hTK2 treated Tk2^{-/-} mice. There was severe mtDNA depletion in kidney tissue of the same treated mutant mice. See FIG. 9.

### Example 7—Treatment with AAV9-hTK2 Ameliorated Biochemical Abnormalities in Brain

Respiratory chain enzyme (RCE) activities and protein levels were measured as described in Example 1.

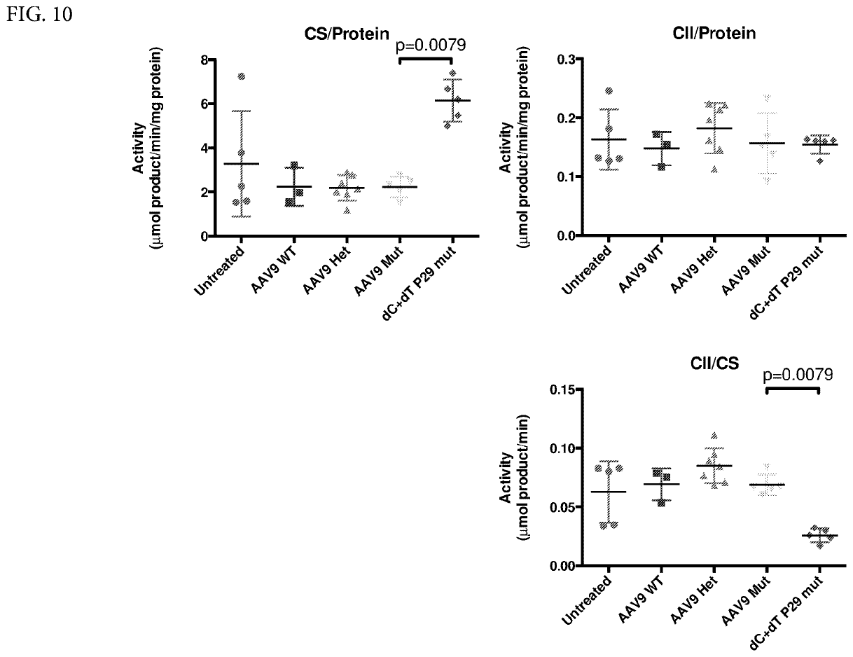

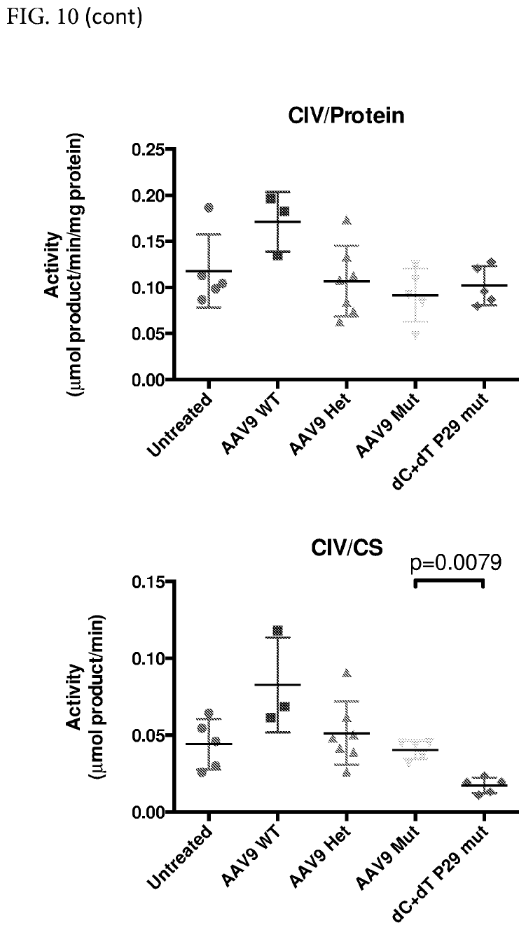

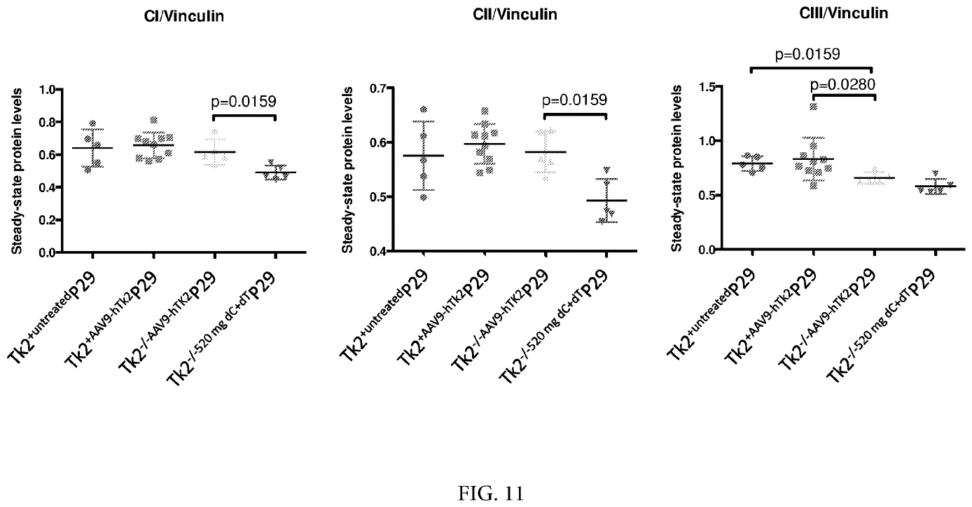

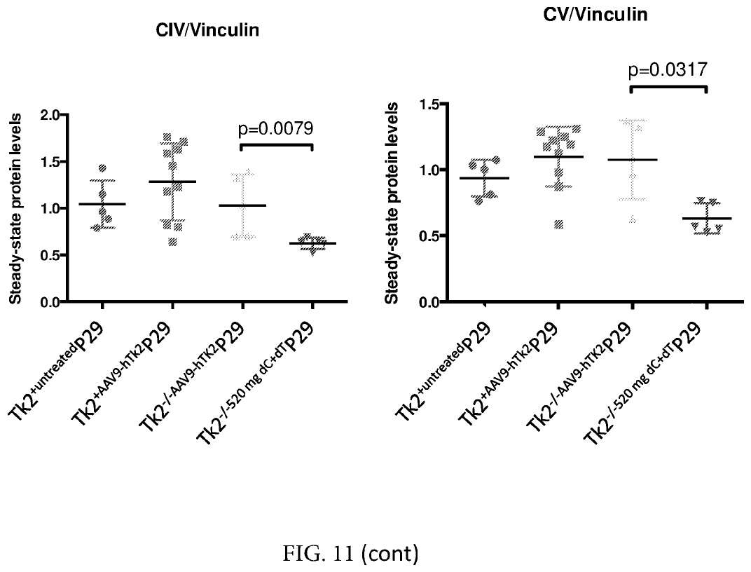

RCE activity in the brain at postnatal day 29 was completely restored in the brains of Tk2^{-/-} mice treated with AAV9-hTK2 (4×10¹¹ vc) (FIG. 10). Only a slight reduction in complex III activity could be observed in AAV9-hTK2 treated Tk2^{-/-} mice compared to untreated wild-type mice (FIG. 11).

### Example 8—AAV9-hTK2 Does Not Rescue Kidney Function

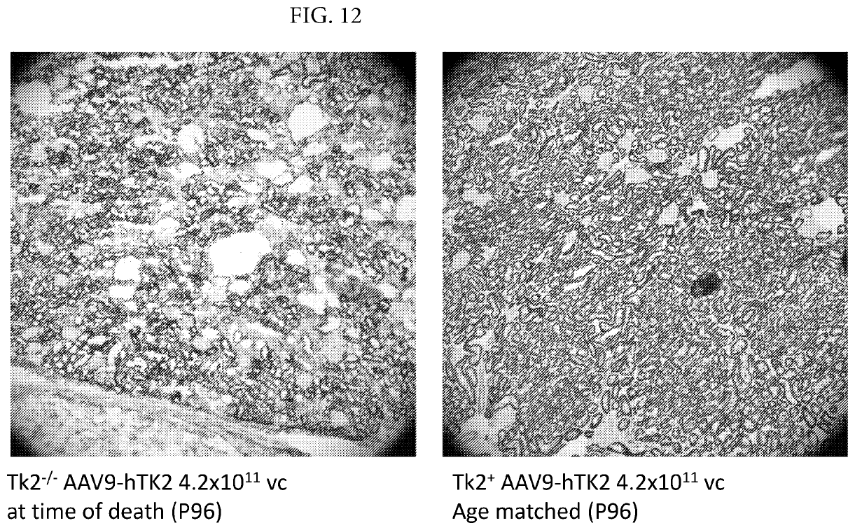

Using the methods in Example 1, kidney tissue of mutant mice treated with AAV9-hTK2 at 4.2×10¹¹ vc at their time of death at postnatal day 96 was stained with SDH and Cox. Kidney tissue of age matched wild-type mice treated with AAV9-hTK2 at 4.2×10¹¹ vc were stained as well. SDH (blue) staining showed no co-localization with Cox (brown) staining indicating Cox deficiency in the kidney tissue of the mutant mice and likely dysfunctional mitochondria (FIG. 12).

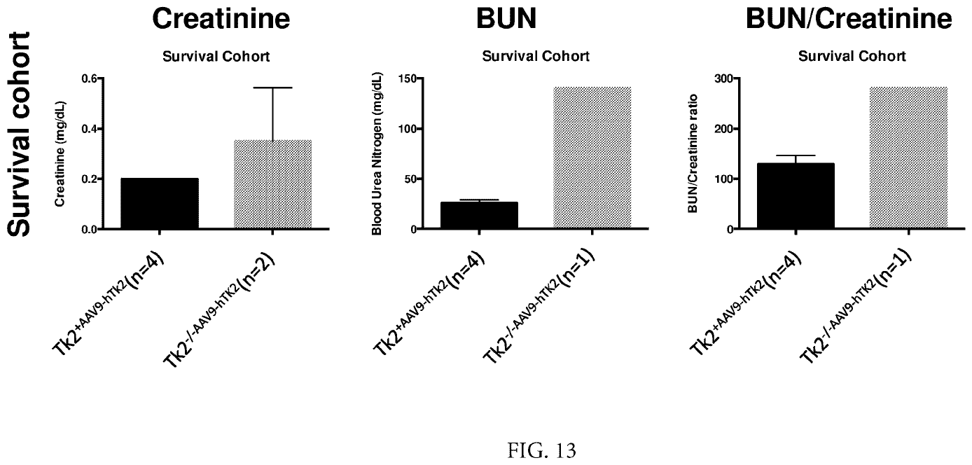

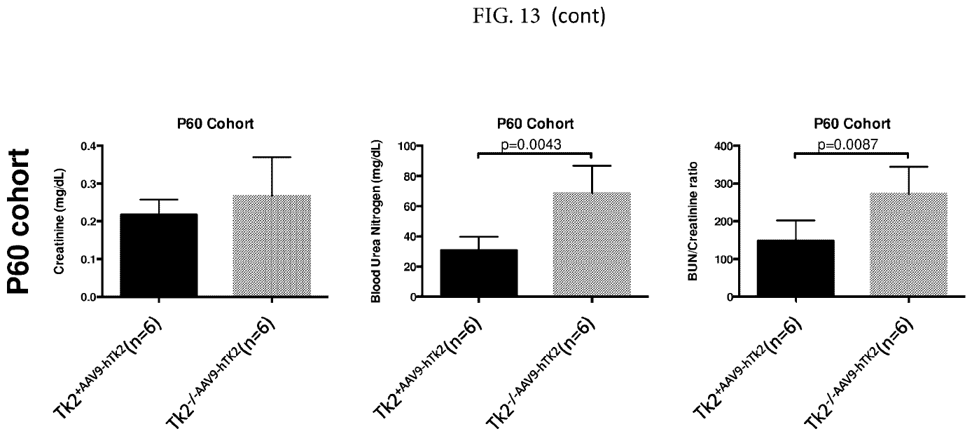

Creatinine and blood urea nitrogen (BUN) index were assessed in both mutant mice treated with AAV9-hTK2 at 4.2×10¹¹ vc and wild-type treated with AAV9-hTK2 at 4.2×10¹¹ vc both in the survival cohort and at postnatal day 60. Both creatinine and BUN were higher in the mutant mice in both cohorts suggesting kidney dysfunction (FIG. 13).

### Example 9—Administration of AAV9-hTK2 in Combination with AAV2-hTK2 Increases Survival Over the Use of AAV9-hTK2 Alone

Mutant mice as described in Example 1 were administered AAV9-hTK2 at 2.1×10¹¹ vc via retro-orbital injection of a total volume of 35 μl as also described in Example 1 at postnatal day 1. Wild-type mice are also treated at postnatal day 1.

At postnatal day 21, one third of these treated mutant and wild-type mice were supplemented with 520 mg/kg/day of oral dC+dT (in drinking water). At postnatal day 29 two thirds of the mice (all of those supplemented with dC+dT and half of the remaining mice) are administered with AAV2-hTK (Example 1) at 1.05×10¹¹ vc, via tail vein injection in a total volume of 100 ul.

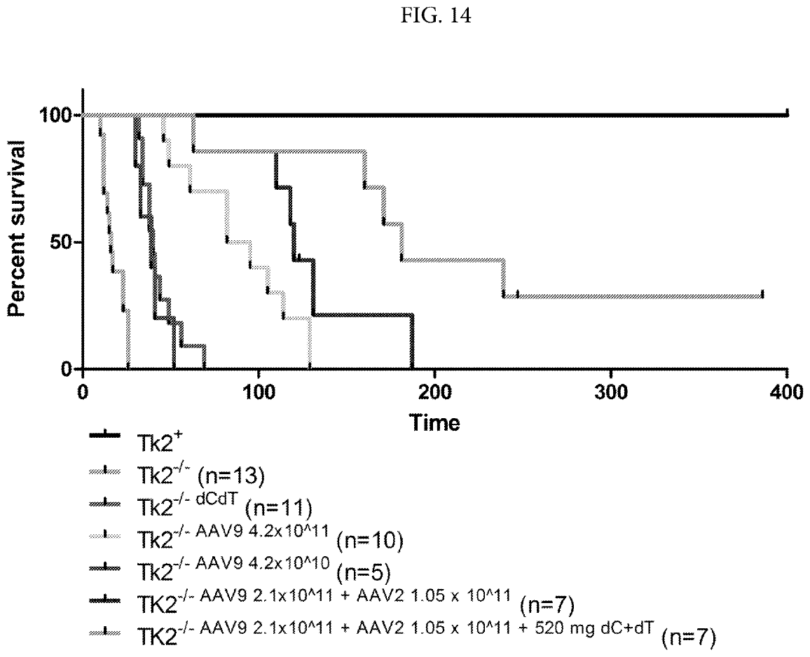

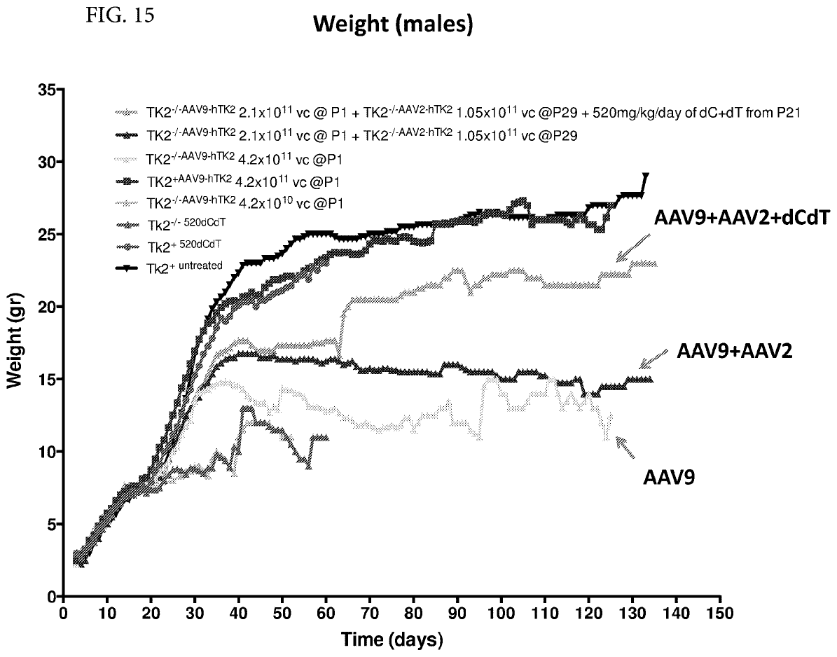

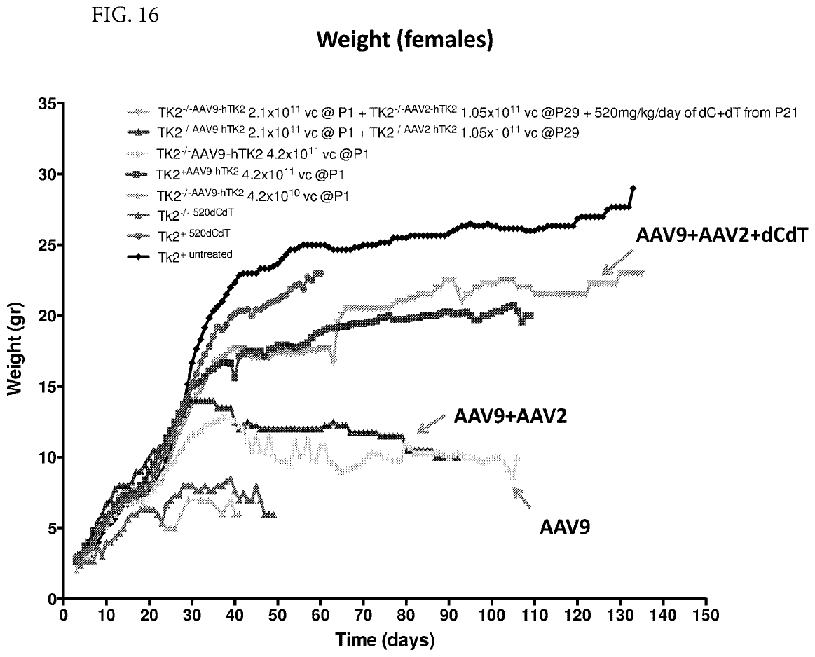

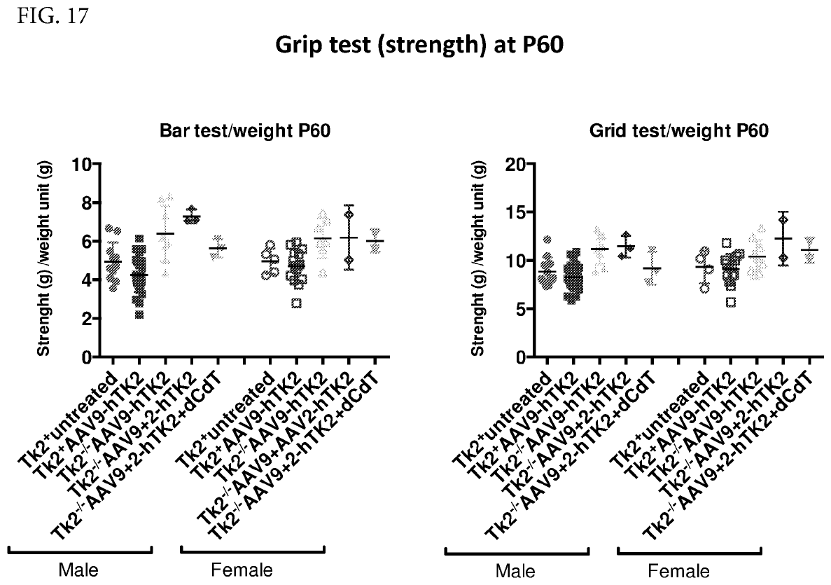

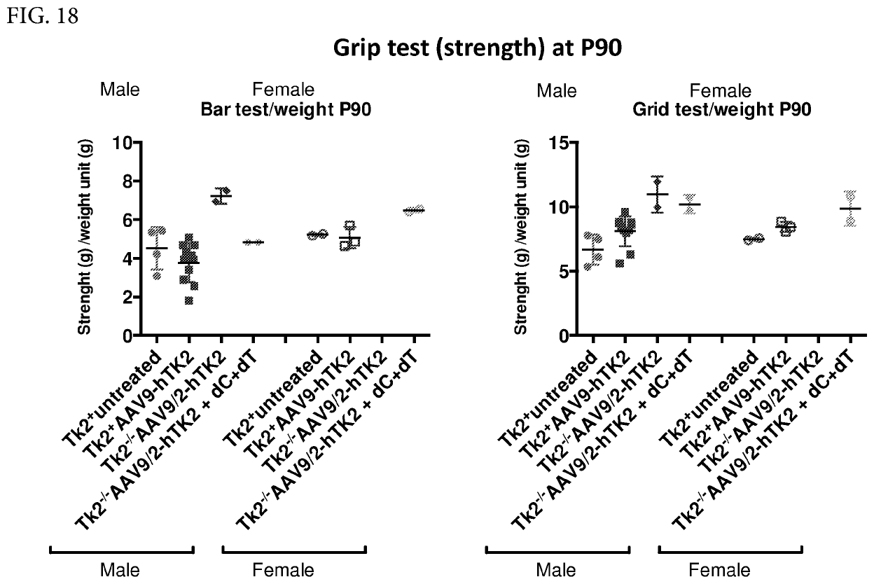

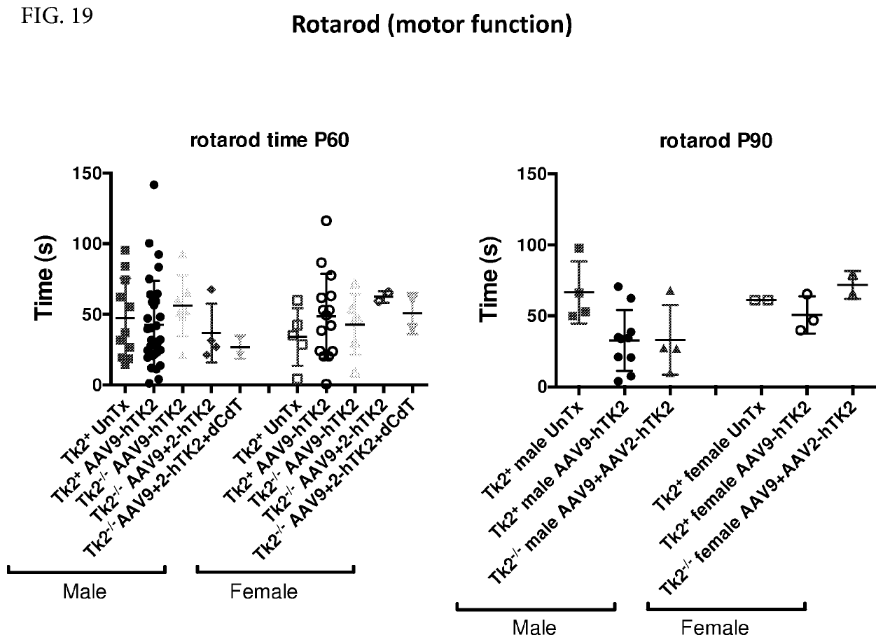

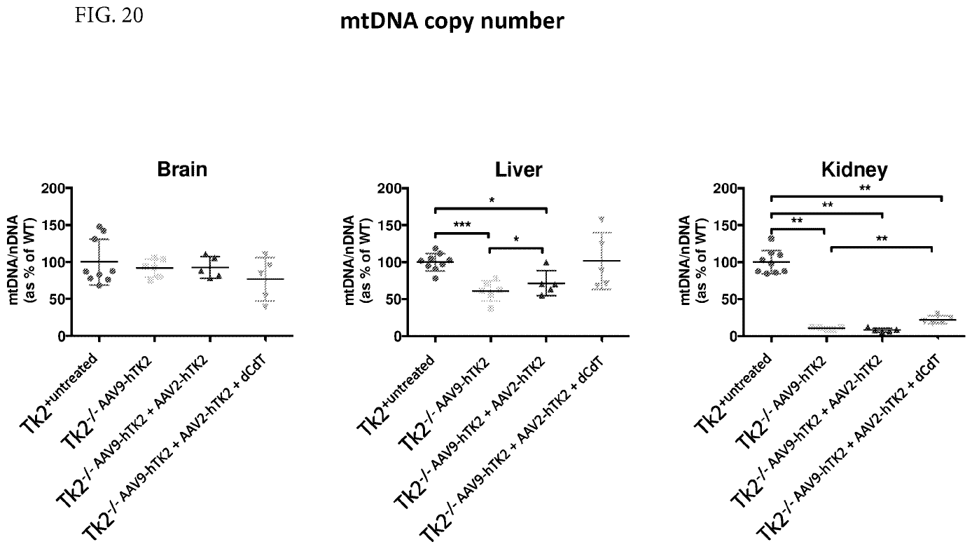

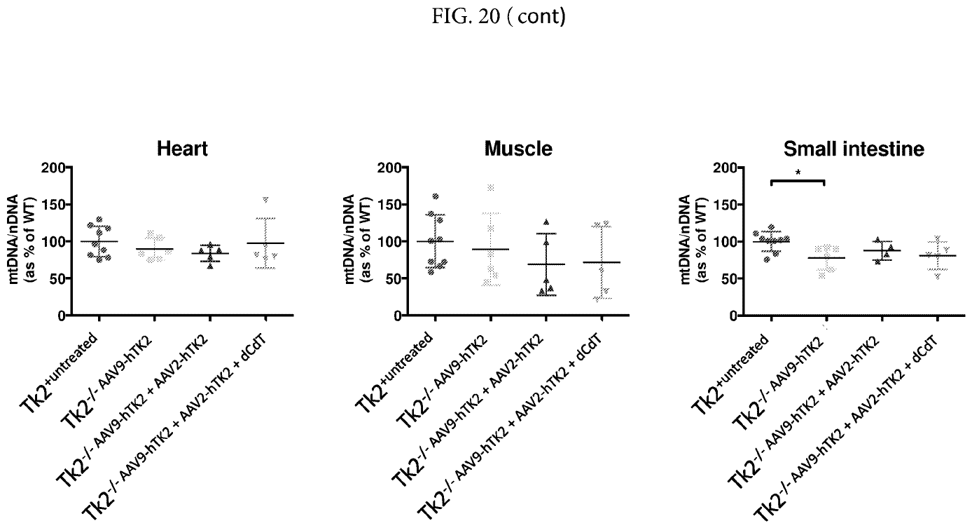

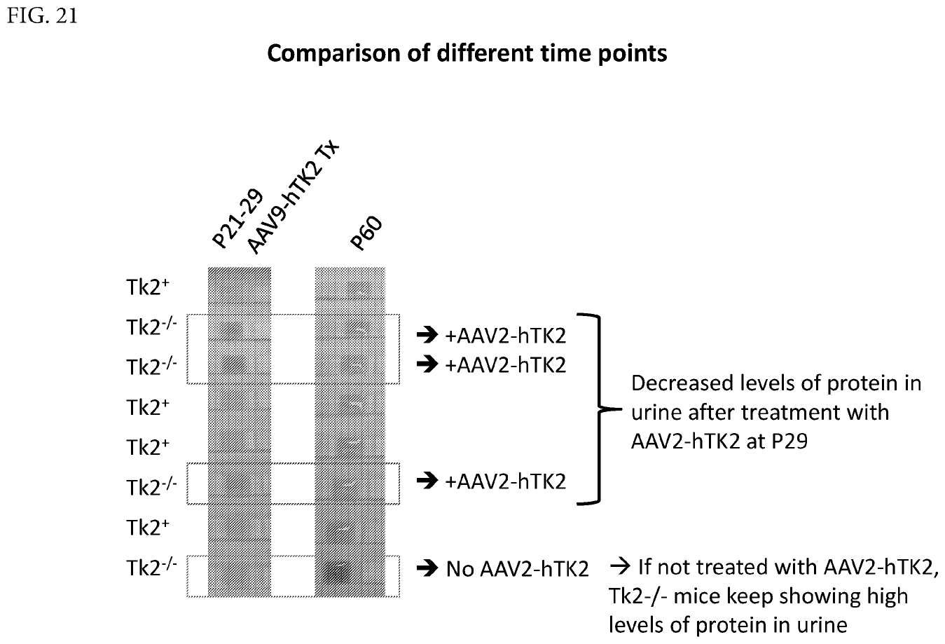

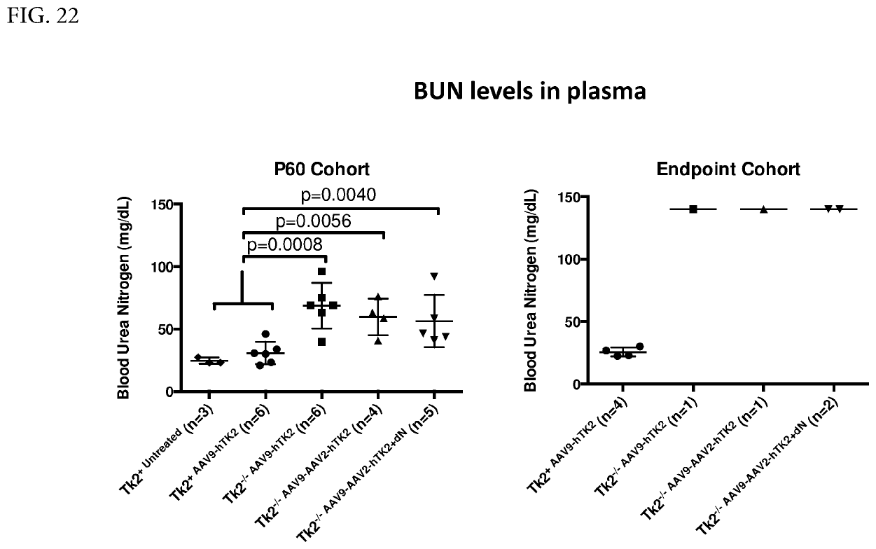

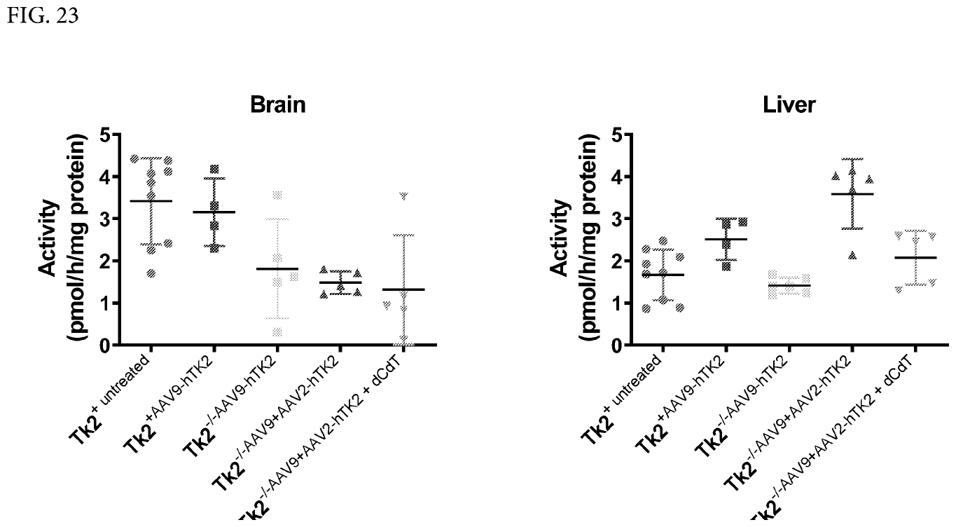

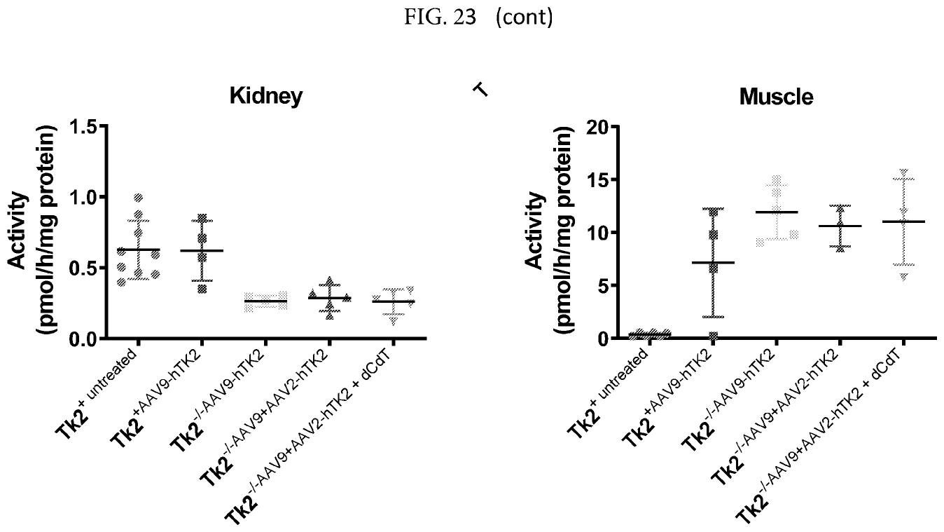

The mutant mice treated with only the AAV9-hTK2 survived an average of 89 days as consistent with the results in Example 2. The mutant mice who received both AAV9-hTK2 and AAV2-hTK2 lived significantly longer, about 120 days (FIG. 14). Despite having an overall lower dose of vector copies (3.15×10¹¹ vc in total versus 4.2×10¹¹ vc), mutant mice treated with both constructs grew as much as mutant mice treated only with AAV9-hTK2 (FIGS. 15 and 16) and showed equal strength and motor coordination (FIGS. 17, 18 and 19). At day 60, mutant mice treated with both AAV9-hTK2 and AAV2-hTK2 showed slightly higher levels of mtDNA than those treated only with AAV9-hTK2 (FIG. 20). Mice treated with a combination of AAV9-hTK2 and AAV2-hTK2 also showed a decrease in the content of protein in the urine (FIG. 21) and slightly lower levels of the index BUN (FIG. 22) as compared to mice treated only with AAV9-hTK2, as well as higher Tk2 activity in liver (FIG. 23).

The combined data on mtDNA, protein in the urine and BUN showed that kidney dysfunction can be delayed in mice co-treated with AAV2 or AAV2+dN which also increases lifespan.

### Example 10—Administration of Supplemental Nucleosides Enhances the Effects of the Gene Therapy

Half of the mutant and wild-type mice treated with both the AAV9-hTK2 and the AAV2-hTK2 in Example 9 were further administered oral dC+dT at 520 mg/kg/day in drinking water as described in Example 1. It is assumed the mice ingest 4 ml a day per mouse.

The mutant mice who were given the supplemental nucleosides survive significantly longer, with a survival of more than 180 days (FIG. 14). They also had increased growth (FIGS. 15 and 16), and equal strength and motor coordination than mice treated with either AAV9-hTK2 or a combination of AAV9-hTK2 and AAV2-hTK2. Mice treated with the combination of AAV9-hTK2 and AAV2-hTK2 and further supplemented with dC+dT showed higher levels of mtDNA in liver.

### Example 11—Administration of AAV9-hTK2 in Combination with dC+dT Increase Survival Over the Use of AAV9-hTK2 Alone

Mutant mice as described in Example 1 are administered oral dC+dT (260 or 520 mg/kg/day each in milk) from postnatal day 4). At postnatal day 21, half the mice are administered with AAV9-hTK2 as described in Example 1.

The mutant mice given only the dC+dT treatment survived a mean of 31 and 40 days with the 260 and 520 mg/kg/day dose, respectively. The mutant mice who received the subsequent AAV9-hTK2 survive significantly longer.

Of the mice that received the AAV9-hTK2 half of these mice then are administered AAV2-hTK2 at postnatal day 30. These mice who received the dC+dT and the AAV9-hTK2 and the AAV2-hTK2 survive significantly longer than the mice receiving only the dC+dT and AAV9-hTK2.

### Example 12—Administration of AAV9-hTK2 in Combination with dCMP+TMP Increase Survival Over the Use of AAV9-hTK2 Alone

Mutant mice as described in Example 1 are administered dCMP+TMP (200 mg/kg/day or 400 mg/kg/day each in milk) from postnatal day 4. At postnatal day 21, half the mice are administered with AAV9-hTK2 as described in Example 1.

The mutant mice given only the dCMP+TMP treatment survive a mean of 35 and 44 days with the 200 and 400 mg/kg/day dose, respectively. The mutant mice who received the subsequent AAV9-hTK2 survive significantly longer.

Of the mice that received the AAV9-hTK2 half of these mice then are administered AAV2-hTK2 at postnatal day 30. These mice who received the dCMP+TMP and the AAV9-hTK2 and the AAV2-hTK2 survive significantly longer than the mice receiving only the dC+dT and AAV9-hTK2.

### REFERENCES

Akman, et al. (2008) Thymidine kinase 2 (H126N) knock in mice show the essential role of balanced deoxynucleotide pools for mitochondrial DNA maintenance. IHum. Mol. Genet. 17:2433-2440
Béhin, et al. (2012) Adult cases of mitochondrial DNA depletion due to TK2 defect An expanding spectrum. Neurologv 78:644-648
Birch-Machn, et at (1994) An evaluation of the measurement of the activities of complexes I-IV in the respiratory chain of human skeletal muscle mitochondria. Biochem MedMetab Bio!51:35-42
Bourdon, et al. (2007) Mutation of RRM2B, encoding p53-controlled ribonucleotide reductase (p53R2), causes severe mitochondrial DNA depletion. Nature Genetics 39:776-780
Chanprasert, et al. (2012) TK2-Related Mitochondrial DNA Depletion Syndrome, Myopathic Form. GeneReviews® Internet, Dec. 6, 2012
Copeland (2008) Inherited mitochondrial diseases of DNA replication. Ann. Rev. iled. 59:131-146
DiMauro, et al. (1987) Cytochrome c oxidase deficiency in Leigh syndrome. Ann. Neurol. 22:498-506
DiMauro, Schon. (2003) Mitochondrial respiratory-chain diseases. New England Journal of Medicine 348:2656-2668
DiMauro, Hirano. (2005) Mitochondrial encephalomyopathies: an update. Neuromuscul. Disord. 15:276-286
Dorado, et al. (2011) Onset and organ specificity of Tk2 deficiency depends on Tk1 down-regulation and transcriptional compensation. Hum. Mol. Genet. 20:155-64
Elpeleg, et al. (2005) Deficiency of the ADP-forming succinyl-CoA synthase activity is associated with encephalomyopathy and mitochondrial DNA depletion. Am. J. Hum. Genet. 76:1081-1086
Franzolin, et al. (2006) Bromovinyl-deoxyuridine: a selective substrate for mitochondrial thymidine kinase in cell extracts. Biochem. Biophy. Res. Comnmun. 344(1):30-6
Galbiati, et al. (2006) New mutations in TK2 gene associated with mitochondrial DNA depletion. Pediatr. Neurol. 34:177-185
Garone, et al. (2012). MPV17 Mutations Causing Adult-Onset Multisysternic Disorder With Multiple Mitochondrial DNA Deletions. Arch Neurol 69:1648-1651
Garone, et al. (2018) Retrospective Natural History of Thymidine Kinase 2 Deficiency. J. Med. Genetics 55:515-21
Gotz, et al. (2008) Thymidine kinase 2 defects can cause multi-tissue mtDNA depletion syndrome. Brain 131:2841-2850
Hirano, et al. (2001) Defects of intergenonic communication: autosomal disorders that cause multiple deletions and depletion of mitochondrial DNA. Semin. Cell. Develop. Biol. 12:417-427
Longley, et al. (2006). Mutant POLG2 disrupts DNA polynerase gamma subunits and causes progressive external ophthalmoplegia. Am J Hum Genet. 78:1026-1034
Mandel, et al. (2001) The deoxyguanosine kinase gene is mutated in individuals with depleted hepatocerebral mitochondrial DNA. Nature Genet. 29:337-341
Naviaux, Nguyen. (2004) POLG mutations associated with Alpers' syndrome and mitochondrial DNA depletion. Ann. Neurol. 55:706-712
Nishino, et al. (1999). Thymidine phosphorylase gene mutations in MNGIE, a human mitochondrial disorder. Science 283:689-692.
Ostergaard, et al. (2007) Deficiency of the alpha subunit of succinate-coenzyme A ligase causes fatal infantile lactic acidosis with mitochondrial DNA depletion. Am. J. Hum. Genet. 81: 383-387
Paradas, et al. (2012) TK2 mutation presenting as indolent myopathy. Neurology 29:504-506
Quinzii, et al. (2013) Tissue-specific oxidative stress and loss of mitochondria in CoQ-deficient Pdss2 mutant mice. FASEB J 27:612-621
Ronchi, et al. (2012). Next-generation sequencing reveals DGUOK mutations in adult patients with mitochondrial DNA multiple deletions. Brain 135:3404-3415.
Saada, et al. (2003) Mitochondrial deoxyribonucleoside triphosphate pools in thynidine kinase 2 deficiency. Biochem. Biophys. Res. Cominun. 310:963-966
Sarzi, et al. (2007) Twinkle helicase (PEO1) gene mutation causes mitochondrial DNA depletion. Ann. Neurol. 62: 579-587
Spelbrink, et al. (2001). Human mitochondrial DNA deletions associated with mutations in the gene encoding Twinkle, a phage T7 gene 4-like protein localized in mitochondria. Nature Genet. 28:223-231
Spinazzola, et al. (2006) MPV17 encodes an inner mitochondrial membrane protein and is mutated in infantile hepatic mitochondrial DNA depletion. Nature Genet. 38:570-575
Tyynismaa, et al. (2012) Thynidine kinase 2 mutations in autosomal recessive progressive external ophthalmoplegia with multiple mitochondrial DNA deletions. Hum. Mol. Genet. 21:66-75
Tyynismaa, et al. (2009). A heterozygous truncating mutation in RRM2B causes autosomal-dominant progressive external ophthalmoplegia with multiple mtDNA deletions. Am. J. Hum. Genet. 85: 290-295
Van Goethern, et al. (2001) Mutation ofPOLG is associated with progressive external ophthalmoplegia characterized by mtDNA deletions. Nature Genet. 28:211-212.

1. A method of treating, preventing, and/or curing a disease or disorder characterized by unbalanced nucleotide pools in a subject in need thereof, the method comprising administering to the subject a therapeutically effective amount of a composition comprising a transgene encoding thymidine kinase 2 (TK2), deoxyguanosine kinase (dGK), thymidine phosphorylase (TP), p53 inducible small subunit of ribonucleotide reductase (p53R2), succinyl-CoA ligase ADP-forming subunit beta (SUCLA2), succinyl-CoA ligase GDP-forming subunit alpha (SUCLG1), mitochondrial inner membrane protein MPV17 (MPV17), and/or DNA polymerase subunit gamma (POLG).

2. A method of restoring enzyme activity in a subject having a disease or disorder characterized by unbalanced nucleotide pools, the method comprising administering to the subject a therapeutically effective amount of a composition comprising a transgene encoding TK2, dGK, TP, p53R2, SUCLA2, SUCLG1, MPV17, and/or POLG.

3. A method of alleviating one or more symptoms associated with a disease or disorder characterized by unbalanced nucleotide pools in a subject in need thereof, the method comprising administering to the subject a therapeutically effective amount of a composition comprising a transgene encoding TK2, dGK, TP, p53R2, SUCLA2, SUCLG1, MPV17, and/or POLG.

4. The method of claim 1, wherein the disease or disorder is a mitochondrial disease.

5. The method of claim 4, wherein the mitochondrial disease is a mitochondrial DNA depletion syndrome (MDS).

6. The method of claim 5, wherein the MDS is a myopathic MDS characterized by one or more mutations in an endogenous gene encoding TK2.

7. The method of claim 5, wherein the MDS is an encephalomyopathic form characterized by one or more mutations in an endogenous gene encoding SUCLA2.

8. The method of claim 5, wherein the MDS is a neurogastrointestinal encephalopathic form characterized by one or more mutations in an endogenous gene encoding TP.

9. The method of claim 5, wherein the MDS is a hepatopathic form characterized by one or more mutations in an endogenous gene encoding dGK, MPV17, and/or POLG.

10. The method of claim 1, wherein the transgene encodes TK2.

11. The method of claim 10, wherein the TK2 has an amino acid sequence that is at least 85% identical to the amino acid sequence of SEQ ID NO: 1.

12. The method of claim 11, wherein the TK2 has an amino acid sequence that is at least 90% identical to the amino acid sequence of SEQ ID NO: 1.

13. The method of claim 12, wherein the TK2 has an amino acid sequence that is at least 95% identical to the amino acid sequence of SEQ ID NO: 1.

14. The method of claim 13, wherein the TK2 has the amino acid sequence of SEQ ID NO: 1.

15. The method of claim 10, wherein the transgene has a nucleic acid sequence that is at least 70% identical to the nucleic acid sequence of SEQ ID NO: 2.

16. The method of claim 15, wherein the transgene has a nucleic acid sequence that is at least 85% identical to the nucleic acid sequence of SEQ ID NO: 2.

17. The method of claim 16, wherein the transgene has a nucleic acid sequence that is at least 95% identical to the nucleic acid sequence of SEQ ID NO: 2.

18. The method of claim 17, wherein the transgene has the nucleic acid sequence of SEQ ID NO: 2.

19. The method of claim 1, wherein the transgene encodes dGK.

20. The method of claim 19, wherein the dGK has an amino acid sequence that is at least 85% identical to the amino acid sequence of SEQ ID NO: 3.

21-176. (canceled)
